(19) United States
(12) Patent Application Publication
(10) Pub. No.: US 2004/0014955 A1
(43) Pub. Date: Jan. 22, 2004

(54) Identification of essential genes of cryptococcus neoformans and methods of use

(76) Inventors: Carlos Zamudio, La Jolla, CA (US); Alexey M. Eroshkin, San Diego, CA (US)

Correspondence Address: PENNIE AND EDMONDS, 1155 AVENUE OF THE AMERICAS, NEW YORK, NY, 100362711

(21) Appl. No.: 10/320,797

(22) Filed: Dec. 16, 2002

Related U.S. Application Data
(60) Provisional application No. 60/341,261, filed on Dec. 17, 2001.

(51) Int. Cl. C07H 21/02; C07H 21/04

(52) U.S. Cl. 536/23.1

(57) ABSTRACT

The present invention provides C. neoformans genes that are essential and are potential targets for drug screening. The nucleotide sequence of the target genes can be used for various drug discovery purposes, such as expression of the recombinant protein, hybridization assay and construction of nucleic acid arrays. The uses of proteins encoded by the essential genes, and genetically engineered cells comprising modified alleles of essential genes in various screening methods are also encompassed by the invention. The present invention also provides methods and compositions that enable the experimental determination as to whether any gene in the genome of Cryptococcus neoformans is essential, and whether that gene is required for virulence or pathogenicity. The identification of essential genes and those genes critical to the development of virulent infections, provides a basis for the development of screens for new drugs against C. neoformans.

This application claims priority to the U.S. provisional application serial No. 60/341,261, filed Dec. 17, 2001, which is incorporated herein by reference in its entirety.

### 1. INTRODUCTION

The present invention is related to the identification of nucleotide sequences of essential genes of Cryptococcus neoformans, and uses thereof in the development of drug screening assays.

### 2. BACKGROUND OF THE INVENTION

Cryptococcus neoformans is an encapsulated fungal pathogen that is the causative agent of cryptococcosis, a life-threatening meningoencephalitis occurring in immunosuppressed patients, particularly those with HIV infection (e.g., see Harrison (2000) J. Infect. 41:12-17). In addition, particular C. neoformans varieties, despite conventional therapy, are associated with significant mortality in immunocompetent individuals and not infrequently can lead to blindness in survivors (Seaton et al., (1996) Q J Med 89:423-428). C. neoformans usually grows in a yeast form on most rich media and in human tissues, and its genome is normally haploid.

C. neoformans appears in at least two varieties: C. neoformans var. neoformans with world-wide distribution and responsible for the vast majority of HIV-associated infections; and C. neoformans var. gattii confined to the tropics and subtropics which causes the above-mentioned infection in immunocompetent individuals. C. neoformans varieties have very large capsules, which comprise the capsular polysaccharide glucuronoxylomannan (GXM), that serve as the primary virulence factor for infection. Acapsular mutant strains produced by several laboratories have been found to be avirulent (e.g., see Kozel et al., (1971) Infect. Immun. 3:287-294; Kwon-Chung et al., (1986) Infect. Immun. 51:218-223). C. neoformans is classified into four distinct capsular serotypes, A-D, based on the composition of the capsular polysaccharides of each serotype (e.g., see Cherniak, et al., (1998) Clin Diagn Lab Immunol 5 (2):146-159). C neoformans var. neoformans comprises serotypes A and D whereas C. neoformans var. gattii comprises serotypes B and C.

Cryptococcal ecology and epidemiology are not fully understood. For instance, human-to-human transmission of C. neoformans has been reported only in exceptional cases suggesting exposure to an environmental source as the predominant means of transmission. In support of this suggestion, environmental isolates from particular niches have been demonstrated to be identical to specific, geographical clinical isolates. Environmental niches proposed as sources of human infection include avian excreta for C. neoformans var. neoformans (Currie et al., (1994) J. Clin. Microbiol. 32:1188-1192; Nosanchuk et al., (2000) Ann Intern Med 132:205-208) and debris from eucalyptus trees for C. neoformans var. gattii (Lazera et al., (1998) Med Mycol. 36:119-122).

Moreover, cryptococcal pathogenesis is poorly understood, including the mechanism by which C. neoformans disseminates from its primary site of infection in the lung to the central nervous system where it achieves latency and persistence. Cryptococcal lesions in patients dying of cryptococcosis often consist of massive extracellular collections of organisms, grossly resembling soap suds (e.g., see Casadevall and Perfect (1998) In Cryptococcus neoformans, ASM Press). Interestingly, the polysaccharide capsule has anti-phagocytic properties in vitro and numerous studies have established that the capsule inhibits phagocytosis and that phagocytic cells cannot ingest cells in vitro in the absence of opsonins (e.g., see Kozel (1988) Infect. Dis. 10:S436-S439). The combination of the anti-phagocytic property of the capsule in vitro and the vast number of extracellular organisms has been the basis for the assumption that C. neoformans causes disease by florid extracellular growth in tissues of susceptible hosts (Casadevall and Perfect (1998) Cryptococcus neoformans, ASM Press).

However, strong lines of evidence also suggest that C. neoformans is a facultative, intracellular parasite that proliferates as a free-living saprophyte and also as an intracellular parasite in phagocytic cells (e.g., see Feldmesser et al., (2001) Trends Microbiol. 9:273-278). Evidence in support of this proposition include: C. neoformans is capable of replicating in phagocytic cells in vitro if treated with opsonins (Lee et al., (1995) Lab. Invest. 73:871-879); C. neoformans resides and survives in vitro in an acidic phagolysosome of infected human macrophages (Levitz et al., (1999) Infect. Immun. 67:885-890); long-term-persistence of C. neoformans in a rat model of latent pulmonary infection is associated with residence of cells inside macrophages (Goldman et al., (2000) Infect. Immun. 68:832-838). The precise mechanism of surviving in and establishing a latent infection in phagocytic cells is not known; however, it appears to be different from other intracellular facultative parasites, such as Legionella and Histoplasma spp (e.g., see Feldmesser et al., (2001) Trends Microbiol. 9:273-278).

In addition to the polysaccharide capsule, several factors have been identified as being important for virulence including melanin production (pigment protects against intracellular free radicals in the macrophage and antimicrobicidal defensin peptides; Kwon-Chung et al., (1982) J. Bacteriol. 150:1414-1421; Doering et al., (1999) Med. Mycol. 37:175-181), phospholipase B (involved in phagosome membrane degradation; Cox et al., (2001) Mol. Microbiol. 39:166-175), cAMP-dependent protein kinase (D Souza et al., (2001) Mol. Cell Biol. 21:3179-3191), calcineurin (Odom et al., (1997) EMBO 16(10):2576-89), ADE2 (Perfect et al., (1993) Infect Immun 61:4446-51); and, urease (Cox et al., (2000) Infect. Immun 68:443-448).

Therapeutic treatment of C. neoformans consists of the antimycotic agents, amphotericin B and flucytosine, or the azoles, ketoconazole and fluconazole (Norris, et al., (1993) Infections in Med. 35-39). These treatments are often complicated by existing infections and such treatments have some very severe side effects. Intracellular survival within macrophages may likely explain the inability of current anti-fungal therapies to eradicate this pathogen. Clearly intracellular residence presents challenges with respect to prophylatic and therapeutic treatments, drug delivery and the efficacy of therapeutic agents against C. neoformans cells within macrophages.

A consortium effort to determine the nucleotide sequence of C. neoformans genome is underway. The understanding of the biology and pathogenicity of C. neoformans will undoubtedly be facilitated by the determination of the nucleotide sequence of the C. neoformans genome. This pathogenic yeast has a defined sexual cycle involving mating of haploid MATa and MATα strains, which allows classical genetic analysis. Molecular biology approaches, including transformation and gene disruption by homologous recombination, and animal models for studies of virulence are well developed.

However, a need for a better understanding of the ecology of C. neoformans and the epidemiology of human infection remains a challenge and solving it will be essential for primary prevention in high-risk individuals. There is also a growing need to develop anti-fungal compounds that are effective therapeutic agents against the free-living, saprophytic cells, intracellular cells and/or both. The present invention provides gene products that are essential for cellular proliferation in C. neoformans and which can serve as novel drug targets for discovery of therapeutic compounds for the eradication of this opportunistic pathogen.

### 3. SUMMARY OF THE INVENTION

The present invention is directed toward the identification of essential genes of Cryptococcus neoformans, the characterization of the gene products, and the construction of conditional-expression mutants and knock-out mutants of each of those genes. Accordingly, the mutants of the invention provide the experimental determination as to whether the genes are essential, and whether the genes are required for virulence or pathogenicity. The information provided herein forms a basis for the development of high-throughput screens for new drugs against C. neoformans.

In one embodiment of the present invention, a set of essential genes of C. neoformans which are potential targets for drug screening, is identified. Such genes have been identified by sequence comparisons with Candida albicans genes which have been determined experimentally to be essential for growth, survival, or virulence of C. albicans, and/or with Aspergillus fumigatus genes which are predicted to be essential or which have been showed experimentally to be essential for growth, survival or virulence of A. fumigatus. The polynucleotides of the essential genes or virulence genes of a C. neoformans (i.e., the target genes) provided by the present invention can be used for various drug discovery purposes. Without limitation, the polynucleotides can be used to express recombinant protein for characterization, screening or therapeutic use; as markers for host tissues in which the pathogenic organisms invade or reside (either permanently or at a particular stage of development or disease states); to compare with the DNA sequence of C. neoformans to identify duplicated genes or paralogs having the same or similar biochemical activity and/or function; to compare with DNA sequences of other related or distant pathogenic organisms to identify potential orthologous essential or virulence genes; for selecting and making oligomers for attachment to a nucleic acid array for examination of expression patterns at various stages of development and disease states; to raise anti-protein antibodies using DNA immunization techniques; as an antigen to raise anti-DNA antibodies or elicit another immune response; and as a therapeutic agent (e.g., antisense molecules) for acute and chronic infections. Where the polynucleotide encodes a protein which binds or potentially binds to another protein (such as, for example, in a receptor-ligand interaction), the polynucleotide can also be used in assays to identify polynucleotides encoding the other protein with which binding occurs or to identify inhibitors of the binding interaction.

In a specific embodiment, the present invention encompasses nucleic acid arrays (or microarrays) which comprise a plurality of defined nucleotide sequences disposed at identifiable positions in an array on a substrate. The defined nucleotide sequences can comprise nucleic acid molecules, preferably oligonucleotides, complementary to, and capable of hybridizing with, the nucleotide sequences of the essential genes of C. neoformans that are identified to be required for the survival, growth and proliferation of C. neoformans, and/or the unique molecular tags employed to mark each mutant C. neoformans strain. Preferably, the nucleic acid array comprises a plurality of nucleic acid molecules, wherein at least one nucleic acid molecule comprises a nucleotide sequence that is hybridizable to a target nucleotide sequence or a complement thereof, said target nucleotide sequence being selected from the group consisting of SEQ ID NO: 1-361, 1001-1361, and 2001-2361.

In various embodiments, the polypeptides or proteins encoded by the essential genes (i.e. the target gene products) provided by the present invention can also be used in assays to determine biological activity, including its uses as a member in a panel or an array of multiple proteins for high-throughput screening; to raise antibodies or to elicit immune response; as a reagent (including the labeled reagent) in assays designed to quantitatively determine levels of the protein (or its receptor) in biological fluids; as a marker for host tissues in which the pathogenic organisms invade or reside (either permanently or at a particular stage of development or in a disease states); and, of course, to isolate correlative receptors or ligands (also referred to as binding partners) especially in the case of virulence factors. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction, such as but not limited to those involved in invasiveness, and pathogenicity of the pathogenic organism. The structure of target proteins can be studied by techniques like X-ray crystallography to yield data that facilitate the rational design of drugs directed against the target proteins.

In specific embodiments, the present invention is directed to nucleic acid arrays (or microarrays) which comprise a plurality of defined nucleotide sequences disposed at spatially addressable positions on a solid substrate. The defined nucleotide sequences can comprise oligonucleotides complementary to, and capable of hybridizing with, the nucleotide sequences of the essential genes of the diploid pathogenic organism that are required for the growth and survival of the diploid pathogenic organism, the nucleotide sequences of genes contributing to the pathogenicity or virulence of the organism, and/or the unique molecular tags employed to mark each of the mutant strains. The present invention also encompasses protein arrays (or microarrays) which comprise a plurality of peptides or polypeptides of defined amino acid sequences as disclosed herein disposed at spatially addressable positions on a solid substrate. The peptides and polypeptides on the array are capable of binding other biomolecules, such as other proteins, including but not limited to its cognate ligand, antibodies, or fragments thereof, nucleic acid molecules, carbohydrates, lipids, etc.

In yet another embodiment of the present invention, conditional-expression mutants of C. neoformans, including both haploid and diploid strains, which are constructed according to the methods disclosed herein, are provided. The present invention provides C. neoformans mutant strains in which an essential gene is modified by the introduction (e.g., by recombination) of a promoter replacement fragment comprising a heterologous promoter, such that the expression of the essential gene is regulated by the heterologous promoter. In one non-limiting example, expression from the heterologous promoter can be regulated by the presence of a transactivator protein comprising a DNA-binding domain and transcription-activation domain. The DNA-binding domain of this transactivator protein recognizes and binds to a sequence in the heterologous promoter and increases transcription of that promoter. The transactivator protein can be produced in the cell by expressing a nucleotide sequence encoding the protein. Accordingly, the present invention encompasses collections of C. neoformans mutant strains wherein each collection comprises a plurality of strains, each strain containing a different conditional-expression mutant gene.

In the present invention, the gene modified in C. neoformans is an essential gene, which is required for survival, growth, and proliferation of the strain. In a preferred embodiment, these modifications lead to the production of a rapid cidal phenotype in the mutant organisms.

The conditional-expression mutants of C. neoformans, including both haploid and diploid strains, are useful for the detection of antifungal agents effective against C. neoformans. Conditional-expression mutants can be cultured under differential growth conditions in the presence or absence of a test compound. The mutants can also be used to infect host cells, such as macrophages, in cell-based assay. The growth rates in culture or inside host cells are then compared to indicate whether or not the compound is active against a target gene product encoded by the conditionally-expressed gene. In one aspect of this embodiment, the conditionally-expressed gene is substantially underexpressed to provide cells with enhanced sensitivity to compounds active against the gene product expressed by that gene. Alternatively, the conditionally-expressed gene may be substantially overexpressed to provide C. neoformans cells with increased resistance to compounds active against the gene product expressed by the conditional-expression mutant allele of the target gene.

In yet another embodiment of the present invention, the C. neoformans strains constructed according to the methods disclosed are used for the screening of therapeutic agents effective for the treatment of non-infectious diseases in a plant or an animal, such as a human. As a consequence of the similarity of a target's amino acid sequence with a plant or animal counterpart, active compounds so identified may have therapeutic applications for the treatment of diseases in the plant or animal, in particular, human diseases, such as cancers and immune disorders.

The present invention, in other embodiments, further encompasses the use of transcriptional profiling and proteomics techniques to analyze the expression of essential and/or virulence genes of C. neoformans under a variety of conditions, including in the presence of known drugs, or when residing inside a host cell. The information yielded from such studies can be used to uncover the target and mechanism of known drugs, to discover new drugs that act in a similar fashion to known drugs, and to delineate the interactions between gene products that are essential to survival, growth, and proliferation of C. neoformans and that are instrumental to virulence and pathogenicity of C. neoformans.

Any or all of these drug discovery utilities are capable of being developed into a kit for commercialization as research products. The kits may comprise polynucleotides and/or polypeptides corresponding to a plurality of Cryptococcus neoformans essential genes of the invention, antibodies, and/or other reagents.

### 4. BRIEF DESCRIPTION OF THE DRAWINGS

FIGS. 1A-1H: A list of the SEQ ID numbers of the genomic and cDNA sequences of the essential genes of Cryptococcus neoformans of the invention, the encoded polypeptides, and the designations of the Candida albicans homologs. Table 1.

### 5. DETAILED DESCRIPTION OF THE INVENTION

### 5.1 Identification of Cryptococcus neoformans Essential Genes

5.1.1 DNA Sequence Analysis of the Cryptococcus neoformans Genome

The present invention provides the nucleotide sequence of essential genes of Cryptococcus neoformans. The essential genes of the invention are identified by comparison of nucleotide sequences of Cryptococcus neoformans genomic DNA and the nucleotide sequences of known essential genes of Candida albicans. Prior to this invention, the essentiality of these C. neoformans genes with respect to the survival, growth, and proliferation of C. neoformans are not known.

The set of nucleotide sequence data used in the present invention has an estimated six times coverage of the Cryptococcus neoformans genome. The nucleotide sequences were annotated by software programs, such as Gene Wise (The Sanger Institute), which can identify coding regions, introns, and splice junctions. Automated as well as manual analysis and curation of the nucleotide sequences were performed to refine and establish precise characterization of the coding regions and other gene features. The nucleotide sequences of Cryptococcus neoformans essential genes were identified by comparisons made with the translated amino acid sequences of known essential genes of C. albicans. The identified C. neoformans genes display a 30% DNA sequence similarity, and/or a 35% amino acid sequence similarity with the corresponding C. albicans sequences. The nucleotide sequences of more than three hundred C. neoformans essential genes are provided in the attached sequence listing and cross-referenced in Table 1 with the identifiers of their homologs in Candida albicans.

To facilitate correlation of the nucleotide sequences of each essential gene with its corresponding amino acid sequence and other related sequences, the sequence identifiers have been organized into four blocks, each with one thousand SEQ ID numbers. Each block of SEQ ID numbers, which corresponds to a type of sequence, has 361 sequences with SEQ ID NOs., and 638 SEQ ID NOs. with no sequence which serve as place holders. Accordingly, the SEQ ID NO. for each of the four related sequences of an essential gene are separated by 1000. For example, SEQ ID NO: 1, 1001, 2001, and 3001, are directed to, respectively, the genomic sequences, the nucleotide sequences of coding regions with introns, the nucleotide sequences of the open reading frame, and the amino acid sequence of one essential gene, and in this example, the Cryptococcus neoformans essential gene is one that is homologous to C. albicans gene CaYLR147C.

To identify and characterize the essential genes of the invention, the inventors assisted by computer algorithms performed searches in computer databases and comparative sequence analysis. The results of such analyses are curated and stored in or displayed on a computer. Such computerized tools for analyzing sequence information are very useful in determining the relatedness of structure of genes and gene products with respect to other genes and gene products in the same species or a different species, and may provide putative functions to novel genes and their products. Biological information such as nucleotide and amino acid sequences are coded and represented as streams of data in a computer.

As used here, the term “computer” includes but is not limited to personal computers, data terminals, computer workstations, networks, computerized storage and retrieval systems, and graphical displays for presentation of sequence information, and results of analyses. Typically, a computer comprises a data entry means, a display means, a programmable processing unit, and a data storage means. A “computer readable medium” can be used to store information such as sequence data, lists, and databases, and includes but is not limited to computer memory, magnetic storage devices, such as floppy discs and magnetic tapes, optical-magnetic storage devices, and optical storage devices, such as compact discs. Accordingly, the present invention also encompass a computer or a computer readable medium that comprises at least one nucleotide sequence selected from the group consisting of SEQ ID NO: 1001-1361 and 2001-2361, or at least one amino acid sequence selected from the group consisting of SEQ ID NO: 3001-3361. In preferred embodiments, the sequences are curated and stored in a form with links to other annotations and biological information associated with the sequences. It is also an object of the invention to provide one or more computers programmed with instructions to perform sequence homology searching, sequence alignment, and structure prediction and model construction, using the nucleotide sequences of the invention, preferably one or more nucleotide sequences selected from the group consisting of SEQ ID NO: 1001-1361 and 2001-2361, and/or one or more amino acid sequence selected from the group consisting of SEQ ID NO: 3001-3361. Computers that comprise, and that can transmit and distribute the nucleotide and/or amino acid sequences of the invention are also contemplated. Also encompassed by the present invention are the uses of one or more nucleotide sequences selected from the group consisting of SEQ ID NO: 1001-1361 and 2001-2361, and/or one or more amino acid sequence selected from the group consisting of SEQ ID NO: 3001-3361 in computer-assisted methods for identifying homologous sequences in public and private sequence databases, in computer-assisted methods for providing putative functional characteristics of a gene product based on structural homology with other gene products with known function(s), in computer-assisted methods of constructing a model of the gene product.

In one specific embodiment, the invention encompasses a method assisted by a computer for identifying a putatively essential gene of a fungus, comprising detecting sequence homology between a fungal nucleotide sequence or fungal amino acid sequence with at least one nucleotide sequence selected from the group consisting of SEQ ID NO: 1001-1361 and SEQ ID NO: 2001-2361, or at least one amino acid sequence selected from the group consisting of SEQ ID NO: 3001-3361.

The features of the nucleotide sequences of the essential genes, the predicted amino acid sequences, nucleic acid arrays, recombinant vectors and expression vectors comprising nucleotide sequences of the C. neoformans essential genes are provided and described hereinbelow in Sections 5.2.1, 5.2.2 and 5.2.3. Genetically engineered yeast cells, prokaryotic cells, and cells of higher eukaryotes comprising nucleotide sequences of the C. neoformans essential genes are provided and described in Section 5.2.3. Antisense nucleic acid molecules corresponding to the C. neoformans essential genes of the invention are provided in Section 5.2.6.

5.1.2. Essentiality of C. neoformans Gene Sequences

In one embodiment of the invention, the inventors analyzed the genomic sequences of C. neoformans and detected a high degree of sequence conservation between C. neoformans sequences and genes in Candida albicans and/or Aspergillus fumigatus. The inventors predicted that these C. neoformans sequences encode functional genes which perform biological functions similar to their homologous counterparts in C. albicans and/or A. fumigatus. Accordingly, the C. neoformans genes identified by the inventors as homologous to essential genes in C. albicans and/or A. fumigatus are predicted to be essential to the survival or growth of C. neoformans.

The essentiality of each of the C. albicans genes used in the sequence homology analysis has been demonstrated experimentally by creating such conditional-expression mutants. Since C. albicans is an obligate diploid organism which comprises two alleles of each gene, one allele of the gene is disrupted or knocked out and the expression of the other allele is placed under the control of a heterologous promoter. The creation and testing of conditional-expression mutants of the C. albicans essential genes are described in PCT publications nos.WO 01/60975 and WO 02/53728, which are both incorporated herein by reference in their entireties. The essentiality of some of the genes used in the sequence homology analysis has also been confirmed experimentally in the Aspergillus fumigatus system.

A C. neoformans gene is considered essential when survival, growth, proliferation and/or viability of an C. neoformans strain is substantially coupled to or dependent on the expression of the gene. An essential function for a cell depends in part on the genotype of the cell and in part the cell's environment. Multiple genes are required for some essential function, for example, energy metabolism, etc. biosynthesis of cell structure, replication and repair of genetic material, etc. Thus, the expression of many genes in an organism are essential for its survival and/or growth. A deletion of or mutation in such a gene resulting in a loss or reduction of its expression and/or biological activity can lead to a loss or reduction of viability or growth of the fungus under normal growth conditions. A deletion of or mutation in an essential gene can cause the C. neoformans cells to perish, stop growing, or display a severe growth defect. The reduction or loss of function of a C. neoformans essential gene can result in cell numbers or growth rate that are in the range of 50%, 40%, 30%, 20%, 10%, 5%, or 1% of that of a wild type C. neoformans under similar conditions. Many essential genes in C. neoformans are expected to contribute to the virulence and/or pathogenicity of the organism. Accordingly, when the virulence and/or pathogenicity of an C. neoformans strain to a defined host, or to a defined set of cells from a host, is associated with the conditional expression of the mutant gene, that essential gene may also be referred to as a virulence gene of C. neoformans.

The essentiality of a gene can be demonstrated by knocking out (insertionally inactivating or deleting) the target gene in C. neoformans and observing the phenotype of the resulting mutant C. neoformans under normal growth conditions and other permissive growth conditions. However, in gene disruption experiments, the observation that a knock-out (e.g. by insertional inactivation or deletion of the target gene) cannot be generated for a gene, cannot, per se, support the conclusion that the gene is an essential gene. Rather, a direct demonstration of expression of the gene in question that is coupled with viability of the cell carrying that gene, is required for the unambiguous confirmation that the gene in question is essential. Accordingly, an essential C. neoformans gene can be placed under the control of a regulatable, heterologous promoter such that a range of expression level of the essential gene in the mutant cell can be obtained. Such levels of expression include negligible or very low expression levels, enabling an evaluation of the phenotype of such a genetically engineered conditional-expression C. neoformans mutant when grown under normal growth conditions and other permissive growth conditions. A loss or reduction of viability or growth of the conditional-expression mutant confirms the essentiality of the C. neoformans gene.

According to the methods of the invention, a collection of conditional-expression mutants of C. neoformans can be generated in which the dosage of specific genes can be modulated, such that their functions in survival, growth, proliferation and/or pathogenicity can be investigated. The information accrued from such investigations allows the identification and validation of individual gene products as potential drug targets. The present invention further provides methods of use of the genetic mutants either individually or as a collection in drug screening and for investigating the mechanisms of drug action.

In another embodiment of the invention, each of the essential genes of the invention represents a potential drug target in C. neoformans that is used individually or as part of a collection, in various methods of screening for drugs active against C. neoformans and other related fungi. Depending on the objective of the drug screening program and the target disease, the essential genes of the invention can be classified and divided into subsets based on the structural features, functional properties, and expression profile of the gene products. The gene products encoded by the essential genes within each subset may share similar biological activity, similar intracellular localization, structural homology, and/or sequence homology. Subsets may also be created based on the homology or similarity in sequence to other organisms in a similar or distant taxonomic group, e.g. homology to Saccharomyces cerevisiae, Schizosaccharomyces pombe genes, or to human genes, or a complete lack of sequence similarity or homology to genes of other organisms, such as S. cerevisiae, S. pombe, or human. Subsets may also be created based on the display of cidal terminal phenotype or static terminal phenotype by the respective C. neoformans mutants. Such subsets, referred to as essential gene sets which can be conveniently investigated as a group in a drug screening program, are provided by the present invention. In a particular embodiment, mutants that display a rapid cidal terminal phenotype are preferred. Moreover, since the products encoded by C. neoformans genes of the invention are involved in biochemical pathways essential to the fungus, analysis of these genes and their encoded products facilitates elucidation of such pathways, thereby identifying additional drug targets. Therefore, the present invention provides a systematic and efficient method for drug target identification and validation. The approach is based on genomics information as well as the biological function of individual genes.

Various methods of use of the C. neoformans nucleotide sequences of the invention in drug target identification and drug screening as described in Section 5.4. Methods of making the gene products of the C. neoformans nucleotide sequences and fragments thereof in prokaryotes, yeasts, and higher eukaryotes, and methods for making antibodies that bind specifically to the gene products and fragments thereof are also encompassed and described in Sections 5.2.3 and 5.2.4.

In yet another embodiment, C. neoformans essential genes can be genetically engineered to be expressed in complementation studies with specific strains of mutant fungal cells, such as Saccharomyces cerevisiae, C. albicans and A. fumigatus mutant cells, that display a loss or reduction of function of the corresponding homologous essential gene. In this manner, an C. neoformans essential gene can be used in complementation studies in a C. albicans, S. cerevisiae or A. fumigatus mutant cell in order to elucidate and establish the structure and function of the gene product of the homologous C. neoformans essential gene.

In a further embodiment, C. neoformans essential gene sequences can also be used to facilitate the creation of a mutant strain of C. neoformans, wherein the C. neoformans essential gene is replaced with the homologous C. albicans gene. Such C. neoformans mutants can be especially useful as C. albicans is an obligate diploid which contains two alleles of every essential gene, and thus requires two molecular events to create a knockout mutant in C. albicans. This C. neoformans mutant allows the expression of a C. albicans essential gene in the cellular background of another pathogen which does not display the respective essential gene function, and can be useful in evaluating the action of potential drug candidates against C. albicans. The same approach can also be used to evaluate drug candidates against A. fumigatus.

Moreover, the present invention provides specifically the use of this information of essentiality to identify orthologs of these essential genes in a non-pathogenic yeast, such as Saccharomyces cerevisiae, and the use of these orthologs in drug screening methods. Although the nucleotide sequence of the orthologs of these essential genes in Saccharomyces cerevisiae may be known, in certain instances, it was not appreciated that these Saccharomyces cerevisiae genes can be useful for discovering drugs against pathogenic fungi, such as C. neoformans.

Furthermore, because of the sequence conservation between gene products of the essential genes of C. neoformans and C. albicans or A. fumigatus, the structure of the gene product of the C. neoformans essential gene can also be used to aid in the rational design of a drug against the homologous C. albicans or A. fumigatus gene product. Thus, in various embodiments, the C. neoformans essential genes can be used in developing drugs that act against C. albicans or other pathogenic fungi. Fungistatic or fungicidal compounds developed by such methods may have a broad host range.

In another embodiment, the invention provides a solid phase comprising one ore more of the target gene products that are present on or immobilized onto at least one surface. Preferably, the target gene products are deposited in a spatially addressable format to form an array (including microarray). Such protein arrays can be used for a variety of purposes, including but not limited to screening for compounds, antibodies, ligands, binding partners, nucleic acids, etc. Preferably, the protein array comprises a plurality of proteins, wherein at least one protein comprises an amino acid sequence or a portion of an amino acid sequence selected from the group consisting of SEQ ID NO:3001 through to SEQ ID NO:3361. The portion of amino acid sequence may comprise at least 20, 30, 40, 50, 60, 70, 80, 90, 100, 150, 200, 250, 300, 350, 400, 450 or more contiguous amino acid residues. Accordingly, fusion proteins, fragments, derivatives, and functionally equivalents of the gene products, including similar proteins from other species, can be present on the array. Methods for making and using such protein arrays are well known in the art, see, for example, U.S. Pat. No. 6,475,808; Sundberg et al. “Spatially-addressable immobiliaztion of macromolecules on solid supports” J. Am. Chem. Soc. 117:12050-12057 (1995); and the articles in “High-Thoughput Proteomics: Protein Arrays”, a supplement to Biotechniques, December 2002 edited by C. Borrebaeck, which are incorporated herein by reference in their entirety.

The biological function of the gene products encoded by the C. neoformans essential genes of the invention can be predicted by the function of their corresponding homologs in yeasts such as C. albicans, and Saccharomyces cerevisiae, and fungi such as A. fumigatus. Accordingly, the C. neoformans genes of the invention may have one or more of the following biological functions:

Metabolism: amino-acid metabolism, amino-acid biosynthesis, assimilatory reduction of sulfur and biosynthesis of the serine family, regulation of amino-acid metabolism, amino-acid transport, amino-acid degradation (catabolism), other amino-acid metabolism activities, nitrogen and sulphur metabolism, nitrogen and sulphur utilization, regulation of nitrogen and sulphur utilization, nitrogen and sulphur transport, nucleotide metabolism, purine-ribonucleotide metabolism, pyrimidine-ribonucleotide metabolism, deoxyribonucleotide metabolism, metabolism of cyclic and unusual nucleotides, regulation of nucleotide metabolism, polynucleotide degradation, nucleotide transport, other nucleotide-metabolism activities, phosphate metabolism, phosphate utilization, regulation of phosphate utilization, phosphate transport, other phosphate metabolism activities, C-compound and carbohydrate metabolism, C-compound and carbohydrate utilization, regulation of C-compound and carbohydrate utilization, C-compound, carbohydrate transport, other carbohydrate metabolism activities, lipid, fatty-acid and isoprenoid metabolism, lipid, fatty-acid and isoprenoid biosynthesis, phospholipid biosynthesis, glycolipid biosynthesis, breakdown of lipids, fatty acids and isoprenoids, lipid, fatty-acid and isoprenoid utilization, regulation of lipid, fatty-acid and isoprenoid biosynthesis, lipid and fatty-acid transport, lipid and fatty-acid binding, other lipid, fatty-acid and isoprenoid metabolism activities, metabolism of vitamins, cofactors, and prosthetic groups, biosynthesis of vitamins, cofactors, and prosthetic groups, utilization of vitamins, cofactors, and prosthetic groups, regulation of vitamins, cofactors, and prosthetic groups, transport of vitamins, cofactors, and prosthetic groups, other vitamin, cofactor, and prosthetic group activities, secondary metabolism, metabolism of primary metabolic sugars derivatives, biosynthesis of glycosides, biosynthesis of secondary products derived from primary amino acids, biosynthesis of amines.

Energy: glycolysis and gluconeogenesis, pentose-phosphate pathway, tricarboxylic-acid pathway, electron transport and membrane-associated energy conservation, accessory proteins of electron transport and membrane-associated energy conservation, other electron transport and membrane-associated energy conservation proteins, respiration, fermentation, metabolism of energy reserves (glycogen, trehalose), glyoxylate cycle, oxidation of fatty acids, other energy generation activities.

Cell Growth, Cell Division and DNA Synthesis: cell growth, budding, cell polarity and filament formation, pheromone response, mating-type determination, sex-specific proteins, sporulation and germination, meiosis, DNA synthesis and replication, recombination and DNA repair, cell cycle control and mitosis, cell cycle check point proteins, cytokinesis, other cell growth, cell division and DNA synthesis activities.

Transcription: rRNA transcription, rRNA synthesis, rRNA processing, other rRNA-transcription activities, tRNA transcription, tRNA synthesis, tRNA processing, tRNA modification, other tRNA-transcription activities, mRNA transcription, mRNA synthesis, general transcription activities, transcriptional control, chromatin modification, mRNA processing (splicing), mRNA processing (5′-, 3′-end processing, mRNA degradation), 3′-end processing, mRNA degradation, other mRNA-transcription activities, RNA transport, other transcription activities.

Protein Synthesis: ribosomal proteins, translation, translational control, tRNA-synthetases, other protein-synthesis activities.

Protein Destination: protein folding and stabilization, protein targeting, sorting and translocation, protein modification, modification with fatty acids (e.g. myristylation, palmitylation, farnesylation), modification by phosphorylation, dephosphorylation, modification by acetylation, other protein modifications, assembly of protein complexes, proteolysis, cytoplasmic and nuclear degradation, lysosomal and vacuolar degradation, other proteolytic degradation, other proteolytic proteins, other protein-destination activities.

Transport Facilitation: channels/pores, ion channels, ion transporters, metal ion transporters (Cu, Fe, etc.), other cation transporters (Na, K, Ca, NH₄, etc.), anion transporters (Cl, SO₄, PO₄, etc.), C-compound and carbohydrate transporters, other C-compound transporters, amino-acid transporters, peptide-transporters, lipid transporters, purine and pyrimidine transporters, allantoin and allantoate transporters, transport ATPases, ABC transporters, drug transporters, other transport facilitators

Cellular Transport and Transport Mechanisms: nuclear transport, mitochondrial transport, vesicular transport (Golgi network, etc.), peroxisomal transport, vacuolar transport, extracellular transport (secretion), cellular import, cytoskeleton-dependent transport, transport mechanism, other transport mechanisms, other intracellular-transport activities.

Cellular Biogenesis: biogenesis of cell wall (cell envelope), biogenesis of plasma membrane, biogenesis of cytoskeleton, biogenesis of endoplasmatic reticulum, biogenesis of Golgi, biogenesis of intracellular transport vesicles, nuclear biogenesis, biogenesis of chromosome structure, mitochondrial biogenesis, peroxisomal biogenesis, endosomal biogenesis, vacuolar and lysosomal biogenesis, other cellular biogenesis activities.

Cellular Communication/signal Transduction: intracellular communication, unspecified signal transduction, second messenger formation, regulation of G-protein activity, key kinases, other unspecified signal transduction activities, morphogenesis, G-proteins, regulation of G-protein activity, key kinases, other morphogenetic activities, osmosensing, receptor proteins, mediator proteins, key kinases, key phosphatases, other osmosensing activities, nutritional response pathway, receptor proteins, second messenger formation, G-proteins, regulation of G-protein activity, key kinases, key phosphatases, other nutritional-response activities, pheromone response generation, receptor proteins, G-proteins, regulation of G-protein activity, key kinases, key phosphatases, other pheromone response activities, other signal-transduction activities.

Cell Rescue, Defense, Cell Death and Ageing: stress response, DNA repair, other DNA repair, detoxificaton, detoxification involving cytochrome P450, other detoxification, cell death, ageing, degradation of exogenous polynucleotides, other cell rescue activities.

Ionic Homeostasis: homeostasis of cations, homeostasis of metal ions, homeostasis of protons, homeostasis of other cations, homeostasis of anions, homeostasis of sulfates, homeostasis of phosphate, homeostasis of chloride, homeostasis of other anions.

Cellular Organization (proteins are localized to the corresponding organelle): organization of cell wall, organization of plasma membrane, organization of cytoplasm, organization of cytoskeleton, organization of centrosome, organization of endoplasmatic reticulum, organization of Golgi, organization of intracellular transport vesicles, nuclear organization, organization of chromosome structure, mitochondrial organization, peroxisomal organization, endosomal organization, vacuolar and lysosomal organization, inner membrane organization, extracellular/secretion proteins.

Since methods for studying the above-listed functions are well known in the art, the predicted biological function of each of the gene products can be readily verified by one of ordinary skill in the art using reagents and cells described herein.

### 5.2. Essential Genes of C. neoformans

5.2.1 Nucleic Acid Molecules, Vectors, and Host Cells

Described herein are the nucleic acid molecules of the invention which encompass a collection of essential genes of C. neoformans.

As used herein, the terms “gene” and “recombinant gene” refer to nucleic acid molecules or polynucleotides comprising a nucleotide sequence encoding a polypeptide or a biologically active ribonucleic acid (RNA). The term can further include nucleic acid molecules comprising upstream, downstream, and/or intron nucleotide sequences. The term “open reading frame (ORF),” means a series of nucleotide triplets coding for amino acids without any termination codons and the triplet sequence is translatable into protein using the codon usage information appropriate for a particular organism.

As used herein, the term “target gene” refers to an essential gene useful in the invention, especially in the context of drug screening. Since it is expected that some genes will contribute to virulence and be essential to the survival of the organism, the terms “target essential gene” and “target virulence gene” will be used where it is appropriate. The target genes of the invention may be partially characterized, fully characterized, or validated as a drug target, by methods known in the art and/or methods taught hereinbelow. As used herein, the term “target organism” refers to a pathogenic organism, the essential and/or virulence genes of which are useful in the invention.

The term “nucleotide sequence” refers to a heteropolymer of nucleotides, including but not limited to ribonucleotides and deoxyribonucleotides, or the sequence of these nucleotides. The terms “nucleic acid” and “polynucleotide” are also used interchangeably herein to refer to a heteropolymer of nucleotides, which may be unmodified or modified DNA or RNA. For example, polynucleotides can be single-stranded or double-stranded DNA, DNA that is a mixture of single-stranded and double-stranded regions, hybrid molecules comprising DNA and RNA with a mixture of single-stranded and double-stranded regions. In addition, the polynucleotide can be composed of triple-stranded regions comprising DNA, RNA, or both. A polynucleotide can also contain one or modified bases, or DNA or RNA backbones modified for nuclease resistance or other reasons. Generally, nucleic acid segments provided by this invention can be assembled from fragments of the genome and short oligonucleotides, or from a series of oligonucleotides, or from individual nucleotides, to provide a synthetic nucleic acid.

The term “recombinant,” when used herein to refer to a polypeptide or protein, means that a polypeptide or protein is derived from recombinant (e. g., microbial or mammalian) expression systems. “Microbial” refers to recombinant polypeptides or proteins made in bacterial or fungal (e.g., yeast) expression systems. As a product, “recombinant microbial” defines a polypeptide or protein essentially unaccompanied by associated native glycosylation. Polypeptides or proteins expressed in most bacterial cultures, e. g., E. coli, will be free of glycosylation modifications; polypeptides or proteins expressed in yeast will be glycosylated.

The term “expression vehicle or vector” refers to a plasmid or phage or virus, for expressing a polypeptide from a nucleotide sequence. An expression vehicle can comprise a transcriptional unit, also referred to as an expression construct, comprising an assembly of (1) a genetic element or elements having a regulatory role in gene expression, for example, promoters or enhancers, (2) a structural or coding sequence which is transcribed into mRNA and translated into protein, and which is operably linked to the elements of (1); and (3) appropriate transcription initiation and termination sequences. “Operably linked” refers to a link in which the regulatory regions and the DNA sequence to be expressed are joined and positioned in such a way as to permit transcription, and ultimately, translation. In the case of Candida albicans, due to its unusual codon usage, modification of a coding sequence derived from other organisms may be necessary to ensure a polypeptide having the expected amino acid sequence is produced in this organism. Structural units intended for use in yeast or eukaryotic expression systems preferably include a leader sequence enabling extracellular secretion of translated protein by a host cell. Alternatively, where a recombinant protein is expressed without a leader or transport sequence, it may include an N-terminal methionine residue. This residue may or may not be subsequently cleaved from the expressed recombinant protein to provide a final product.

The term “recombinant host cells” means cultured cells which have stably integrated a recombinant transcriptional unit into chromosomal DNA or carry stably the recombinant transcriptional unit extrachromosomally. Recombinant host cells as defined herein will express heterologous polypeptides or proteins, and RNA encoded by the DNA segment or synthetic gene in the recombinant transcriptional unit. This term also means host cells which have stably integrated a recombinant genetic element or elements having a regulatory role in gene expression, for example, promoters or enhancers. Recombinant expression systems as defined herein will express RNA, polypeptides or proteins endogenous to the cell upon induction of the regulatory elements linked to the endogenous DNA segment or gene to be expressed. The cells can be prokaryotic or eukaryotic.

The term “polypeptide” refers to the molecule formed by joining amino acids to each other by peptide bonds, and may contain amino acids other than the twenty commonly used gene-encoded amino acids. The term “active polypeptide” refers to those forms of the polypeptide which retain the biologic and/or immunologic activities of any naturally occurring polypeptide. The term “naturally occurring polypeptide” refers to polypeptides produced by cells that have not been genetically engineered and specifically contemplates various polypeptides arising from post-translational modifications of the polypeptide including, but not limited to, proteolytic processing, acetylation, carboxylation, glycosylation, phosphorylation, lipidation and acylation.

The term “isolated” as used herein refers to a nucleic acid or polypeptide separated from at least one macromolecular component (e.g., nucleic acid or polypeptide) present with the nucleic acid or polypeptide in its natural source. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

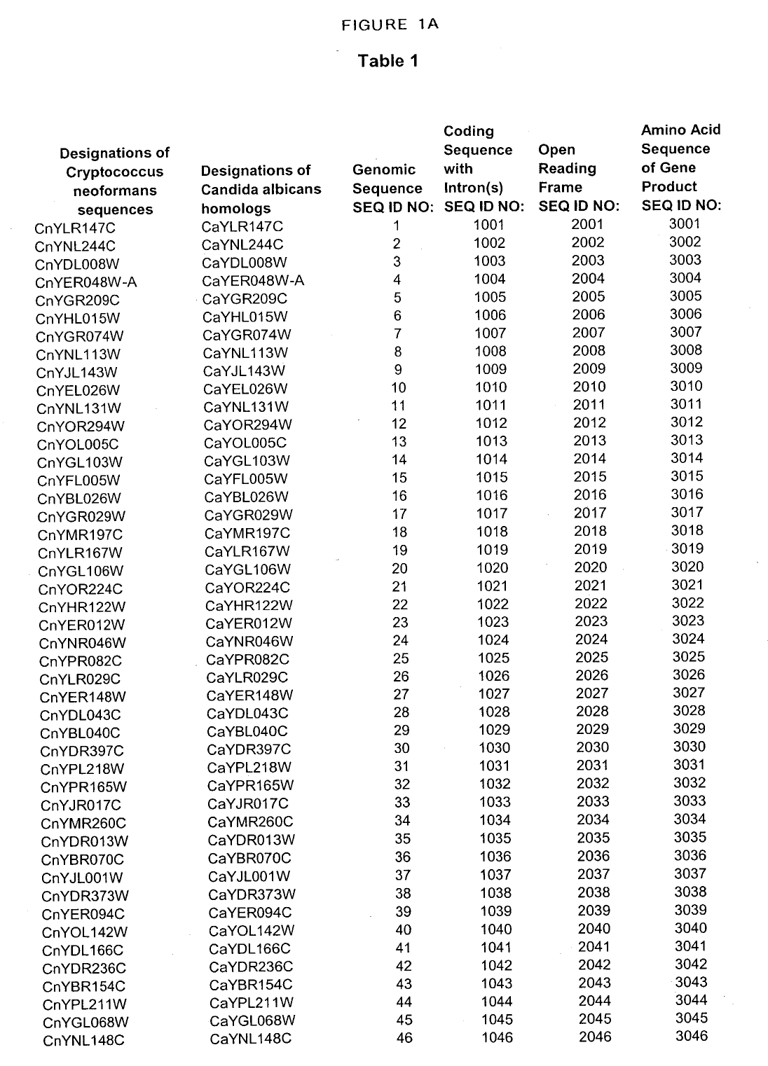

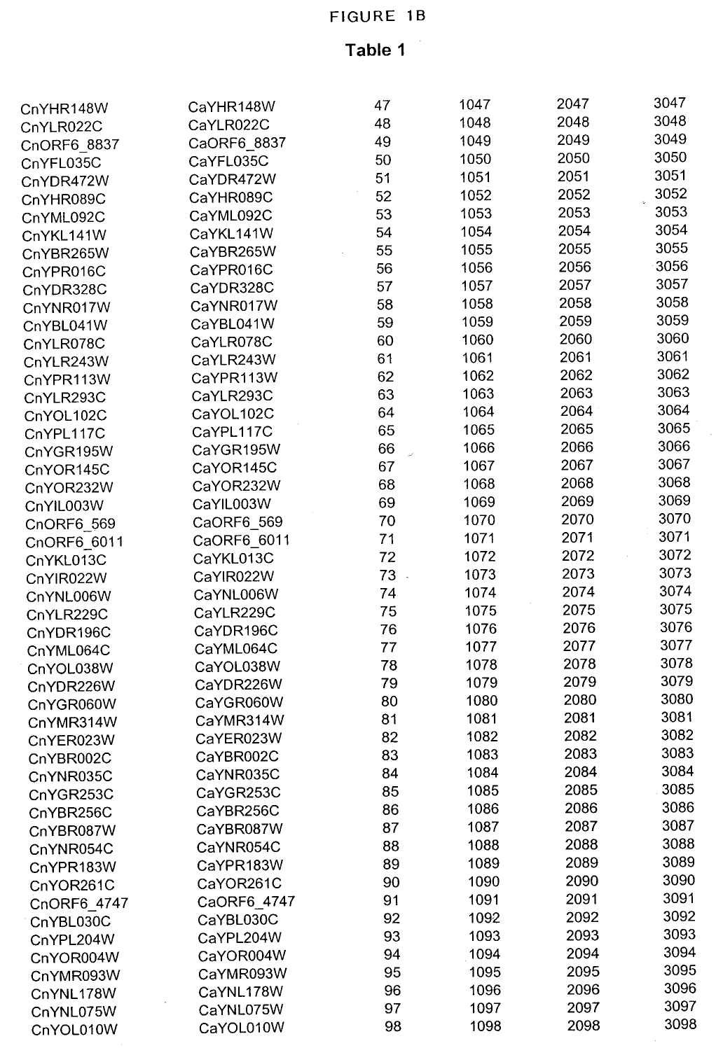

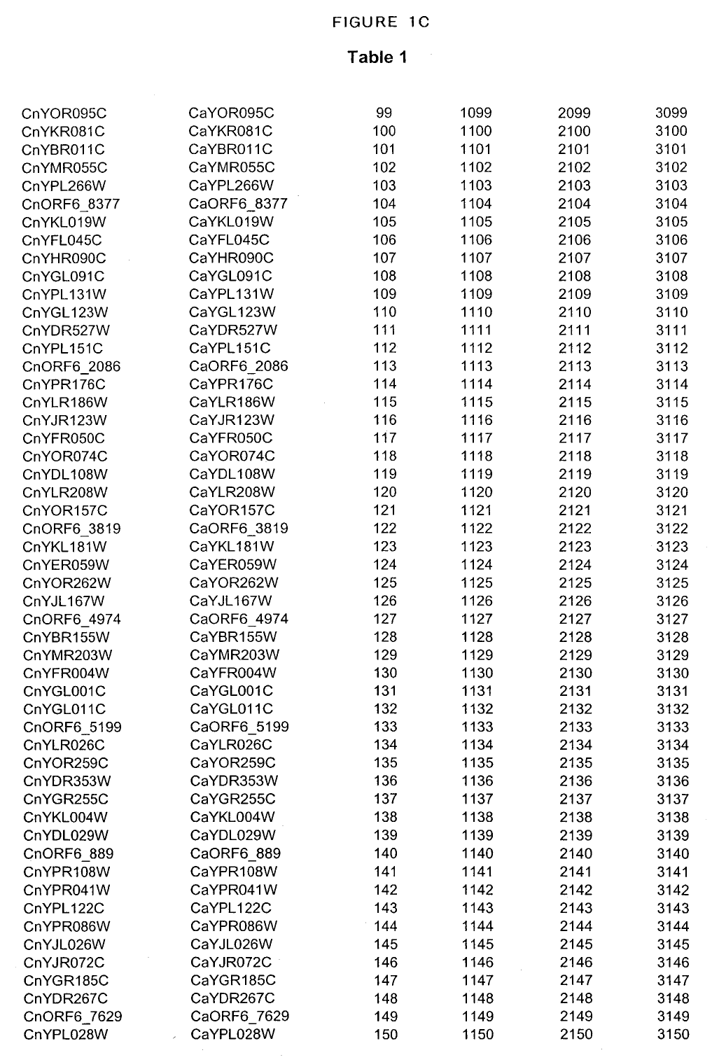

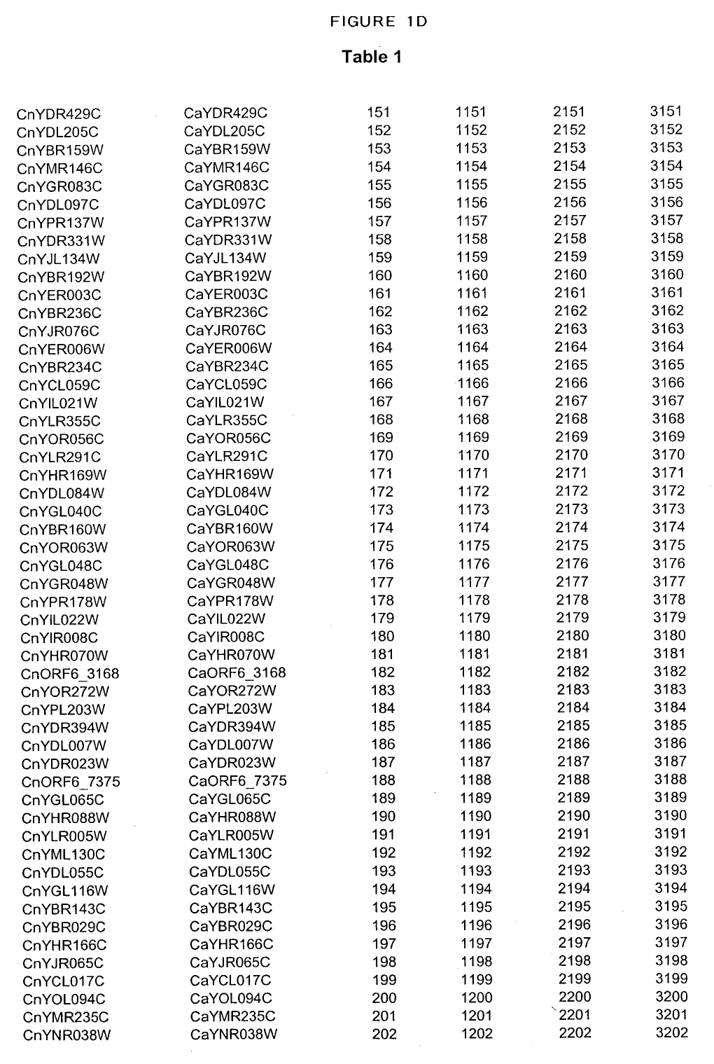

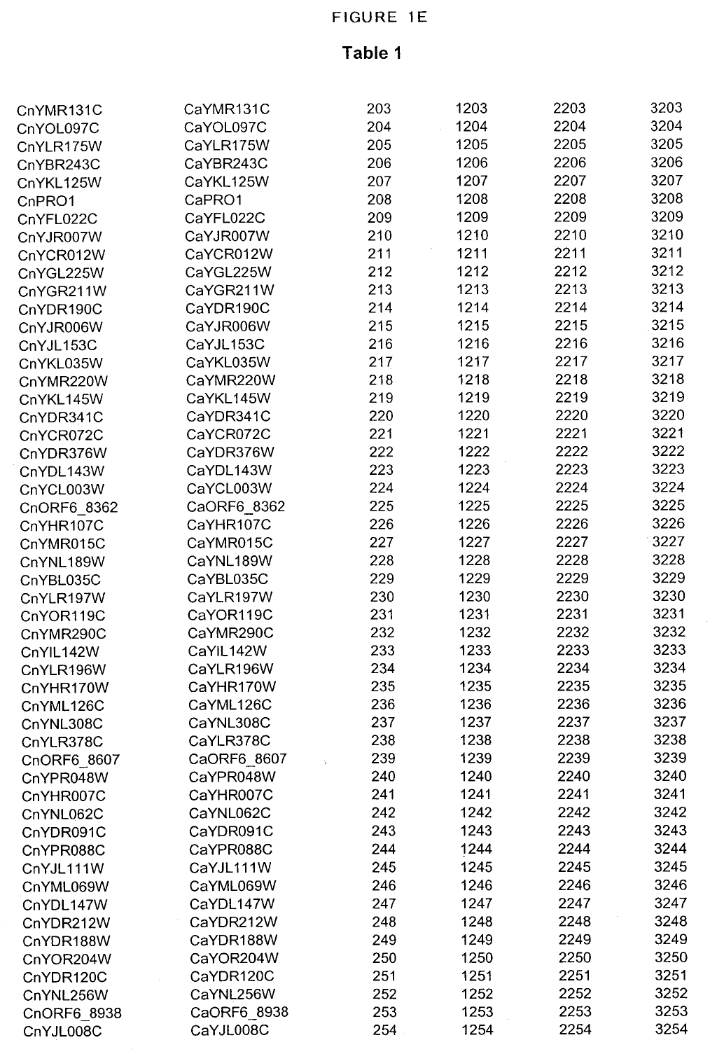

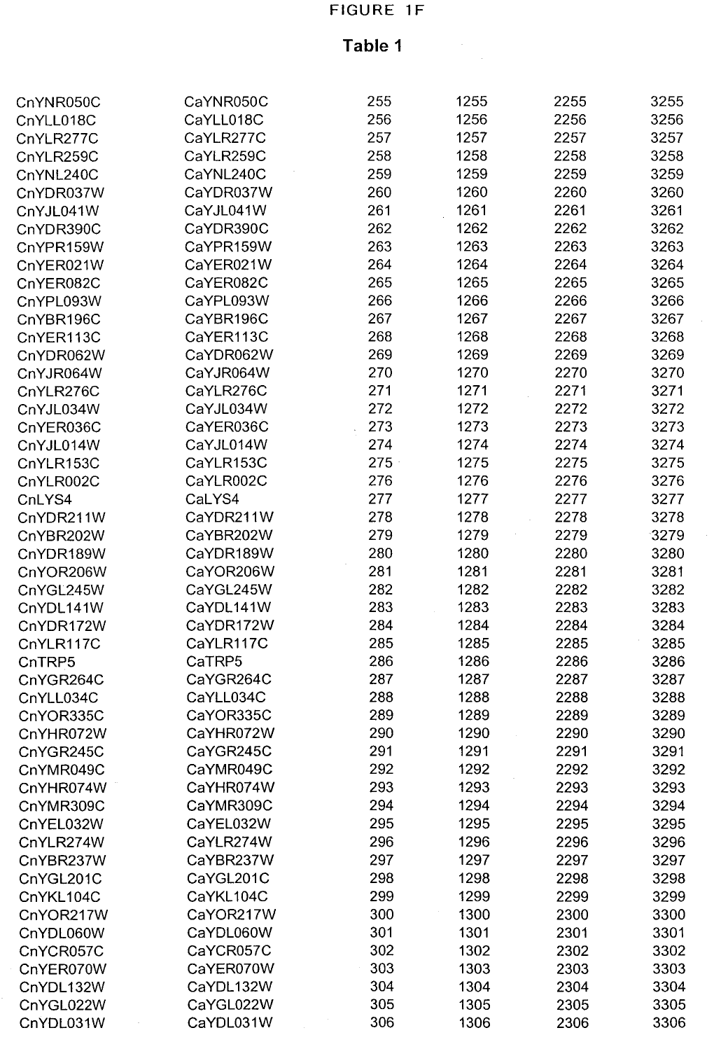

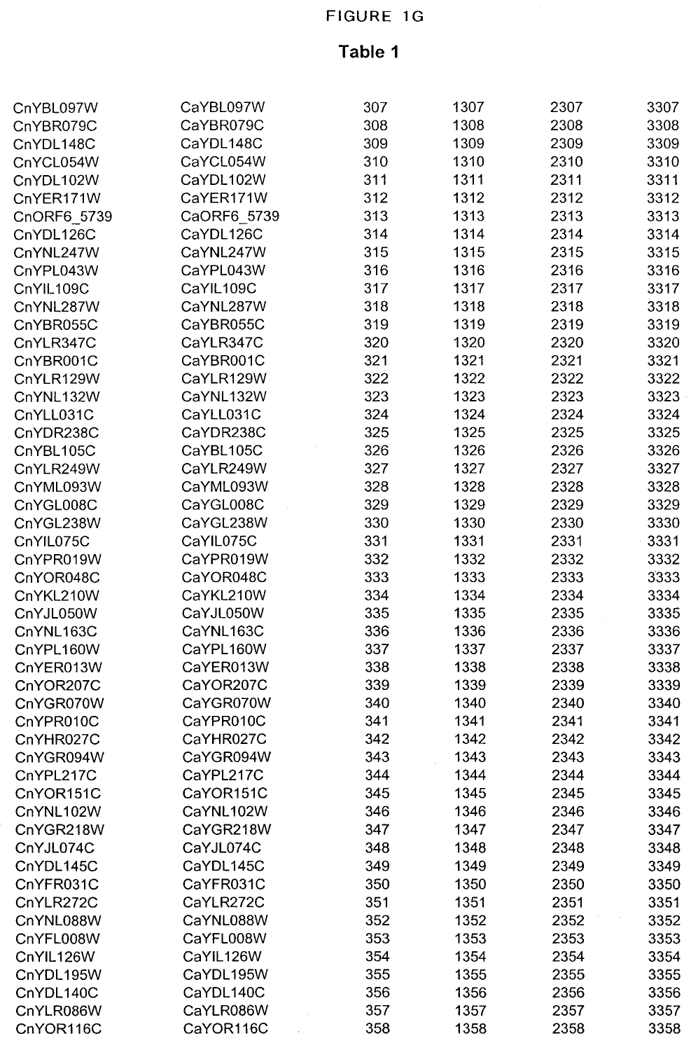

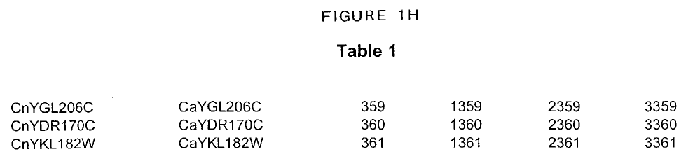

In one embodiment, the present invention provides the identities of more than three hundred essential genes. FIG. 1 lists the sequence identifiers of the genomic nucleotide sequences and coding region of these genes that are essential in C. neoformans and that share a high degree of sequence conservation with the known essential genes of C. albicans and/or A. fumigatus. The genomic sequences including sequences upstream and downstream of the coding regions, the reading frames, the positions of exons and introns of these genes are not previously known. The fact that these genes are essential to the growth and/or survival of C. neoformans was not known until the inventors' discovery. Thus, the uses of these gene sequences and their gene products are encompassed by the present invention. Accordingly, SEQ ID NO: 2001-2361, each identifies a nucleotide sequence of the opening reading frame (ORF) of an identified essential gene. The genomic sequences of the essential genes including sequences upstream and downstream of the coding regions are set forth in SEQ ID NOs: 1-361. The genomic sequences of the essential genes including intron sequences are set forth in SEQ ID NO: 1001-1361. The predicted amino acid sequence of the identified essential genes are set forth in SEQ ID NO: 3001-3361, which are obtained by conceptual translation of the nucleotide sequences of SEQ ID NO: 2001-2361 once the reading frame is determined. In a preferred embodiment, a nucleotide sequence that encodes a product of an essential gene of the invention lacks intron sequences. However, also encompassed are gene products, such as splice variants, that are encoded by the genomic sequences of SEQ ID NO: 1-361 and 1001-1361, and their nucleotide sequences and amino acid sequences.

The DNA sequences were generated by sequencing reactions and may contain minor errors which may exist as misidentified nucleotides, insertions, and/or deletions. However, such minor errors, if present, in the sequence database should not disturb the identification of the ORF as an essential gene of the invention. Since sequences of the ORFs are provided herein and can be used to uniquely identify the corresponding gene in the C. neoformans genome, one can readily obtain a clone of the gene corresponding to the ORFs by any of several art-known methods, repeat the sequencing and correct the minor error(s). The disclosure of the ORFs or a portion thereof essentially provides the complete gene by uniquely identifying the coding sequence in question, and providing sufficient guidance to obtain the complete cDNA or genomic sequence.

Moreover, minor sequence errors and variation in splicing do not affect the construction of conditional-expression C. neoformans mutant strains and the uses of those strains, since these methods do not require absolute sequence identity between the chromosomal DNA sequences and the sequences of the gene in the primers or recombinant DNA. In some instances, the correct reading frame of the C. neoformans gene can be identified by comparing its overall amino acid sequence with known Saccharomyces cerevisiae, Candida albicans and/or Aspergillus fumigatus sequences. Accordingly, the present invention encompasses C. neoformans genes which correspond to the ORFs identified in the invention, polypeptides encoded by C. neoformans genes which correspond to the ORFs identified in the invention, and the various uses of the polynucleotides and polypeptides of the genes which correspond to the ORFs of the invention. As used herein in referring to the relationship between a specified nucleotide sequence and a gene, the term “corresponds” or “corresponding” indicates that the specified sequence effectively identifies the gene. In general, correspondence is substantial sequence identity barring minor errors in sequencing, allelic variations and/or variations in splicing. Correspondence can be a transcriptional relationship between the gene sequence and the mRNA or a portion thereof which is transcribed from that gene. This correspondence is present also between portions of an mRNA which is not translated into polypeptide and DNA sequence of the gene.

The essential genes listed in Table 1 can be obtained using cloning methods well known to those of skill in the art, and include but are not limited to the use of appropriate probes to detect the genes within an appropriate cDNA or gDNA (genomic DNA) library (See, for example, Sambrook et al., 1989, Molecular Cloning: A Laboratory Manual, Cold Spring Harbor Laboratories, which is incorporated herein by reference in its entirety). Probes for the sequences identified herein can be synthesized based on the DNA sequences disclosed herein in SEQ ID NO:1-361, 1001-1361 and 2001-2361. As used herein, “target gene” (e.g. essential and/or virulence gene) refers to (a) a gene containing at least one of the DNA sequences and/or fragments thereof that are set forth in SEQ ID NO: 2001-2361; (b) any DNA sequence or fragment thereof that encodes the amino acid sequence that are set forth in SEQ ID NO: 3001-3361, as well as the gene product encoded by genomic SEQ ID NO: 1-361 and 1001-1361, as expressed by C. neoformans; (c) any DNA sequence that hybridizes to the complement of the nucleotide sequences set forth in SEQ ID NO: 1-361, 1001-1361 and 2001-2361 under stringent conditions, e.g., hybridization to filter-bound DNA in 6×sodium chloride/sodium citrate (SSC) at about 45° C. followed by one or more washes in 0.2×SSC/0.1% SDS at about 50-65° C., or under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel, F. M. et al., eds., 1989, Current Protocols in Molecular Biology, Vol. I, Green Publishing Associates, Inc. and John Wiley & Sons, Inc., New York, at pp. 6.3.1-6.3.6 and 2.10.3). Preferably, the polynucleotides that hybridize to the complements of the DNA sequences disclosed herein encode gene products, e.g., gene products that are functionally equivalent to a gene product encoded by a target gene. As described above, target gene sequences include not only degenerate nucleotide sequences that encode the amino acid sequences of SEQ ID NO: 3001-3361, as well as the gene product encoded by genomic SEQ ID NO: 1-361 and 1001-1361, as expressed in C. neoformans, but also degenerate nucleotide sequences that when translated in organisms other than C. neoformans, would yield a polypeptide comprising one of the amino acid sequences of SEQ ID NO: SEQ ID NO: 3001-3361, as well as the gene product encoded by genomic SEQ ID NO: 1-361 and 1001-1361, as expressed by C. neoformans, or a fragment thereof. One of skill in the art would know how to select the appropriate codons or modify the nucleotide sequences of SEQ ID NO: 2001-2361, when using the target gene sequences in C. neoformans or in other organisms. Moreover, the term “target gene” encompasses genes that are naturally occurring in Saccharomyces cerevisiae, Candida albicans, or Aspergillus fumigatus, or variants thereof, that share extensive nucleotide sequence homology with C. neoformans genes having one of the DNA sequences that are set forth in SEQ ID NO: 2001-2361, i.e., the orthologs in S. cerevisiae, C. albicans or A. fumigatus. It is contemplated that methods for drug screening that can be applied to C. neoformans genes can also be applied to orthologs of the same genes in the non-pathogenic S. cerevisiae and in the pathogenic C. albicans. Thus, the screening methods of the invention are applicable to target genes that, depending on the objective of the screen, may or may not include genes of S. cerevisiae, C. albicans, or A. fumigatus origin.

In another embodiment, the invention also encompasses the following polynucleotides, host cells expressing such polynucleotides and the expression products of such nucleotides: (a) polynucleotides that encode portions of target gene product that corresponds to its functional domains, and the polypeptide products encoded by such nucleotide sequences, and in which, in the case of receptor-type gene products, such domains include, but are not limited to signal sequences, extracellular domains (ECD), transmembrane domains (TM) and cytoplasmic domains (CD); (b) polynucleotides that encode mutants of a target gene product, in which all or part of one of its domains is deleted or altered, and which, in the case of receptor-type gene products, such mutants include, but are not limited to, mature proteins in which the signal sequence is cleaved, soluble receptors in which all or a portion of the TM is deleted, and nonfunctional receptors in which all or a portion of CD is deleted; and (d) polynucleotides that encode fusion proteins containing a target gene product or one of its domains fused to another polypeptide.

The invention also includes polynucleotides, preferably DNA molecules, that hybridize to, and are therefore the complements of, the DNA sequences of the target gene sequences. Such hybridization conditions can be highly stringent or less highly stringent, as described above and known in the art. The nucleic acid molecules of the invention that hybridize to the above described DNA sequences include oligodeoxynucleotides (“oligos”) which hybridize to the target gene under highly stringent or stringent conditions. In general, for oligos between 14 and 70 nucleotides in length the melting temperature (Tm) is calculated using the formula:

Tm(° C.)=81.5+16.6(log [monovalent cations (molar)]+0.41 (% G+C)−(500/N)

where N is the length of the probe. If the hybridization is carried out in a solution containing formamide, the melting temperature may be calculated using the equation:

Tm(° C.)=81.5+16.6 (log [monovalent cations (molar)])+0.41(% G+C)−(0.61) (% formamide)−(500/N).

where N is the length of the probe. In general, hybridization is carried out at about 20-25 degrees below Tm (for DNA-DNA hybrids) or about 10-15 degrees below Tm (for RNA-DNA hybrids). Other exemplary highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos).

These nucleic acid molecules can encode or act as target gene antisense molecules, useful, for example, in target gene regulation and/or as antisense primers in amplification reactions of target gene nucleotide sequences. Further, such sequences can be used as part of ribozyme and/or triple helix sequences, also useful for target gene regulation. Still further, such molecules can be used as components of diagnostic methods whereby the presence of the pathogen can be detected. The uses of these nucleic acid molecules are discussed in detail below.

Fragments of the target genes of the invention can be at least 10 nucleotides in length. In alternative embodiments, the fragments can be about 20, 30, 40, 50, 60, 70, 80, 90, 100, 200, 300, 400, 500, 1000, 1500, 2000, 2500, 3000, 3500, 4000, 4500, 5000 or more contiguous nucleotides in length. Alternatively, the fragments can comprise nucleotide sequences that encode at least 10, 20, 30, 40, 50, 100, 150, 200, 250, 300, 350, 400, 450 or more contiguous amino acid residues of the target gene products. Fragments of the target genes of the invention can also refer to exons or introns of the above described nucleic acid molecules, as well as portions of the coding regions of such nucleic acid molecules that encode functional domains such as signal sequences, extracellular domains (ECD), transmembrane domains (TM) and cytoplasmic domains (CD).

In another embodiment, the present invention encompasses nucleic acid molecules comprising nucleotide sequences of introns of the essential genes of the invention. The nucleotide sequences of one or more introns of each essential gene, where present, are provided by the segment(s) of nucleotide sequences that are present in the genomic sequences (SEQ ID NO: 1001-1361) and that are absent in the corresponding open reading frame sequences (SEQ ID NO: 2001-2361). Nucleic acid molecules comprising these intron sequences or fragments thereof, although not separately provided in the sequence listing, are encompassed, and are useful for a variety of purposes, for example, as oligonucleotide primers for isolating individual exons by polymerase chain reaction or as a diagnostic tool for identifying and/or detecting a strain of C. neoformans.

In another embodiment, the present invention is directed toward the regulatory regions that are found upstream and downstream of the coding sequences disclosed herein, which are readily determined and isolated from the genomic sequences provided herein. Included within such regulatory regions are, inter alia, promoter sequences, upstream activator sequences, as well as binding sites for regulatory proteins that modulate the expression of the genes identified herein.

In addition to the above enumerated uses, the nucleotide sequences of essential genes of C. neoformans have the following specific utilities:

The nucleotide sequences of the invention can be used as genetic markers and/or sequence markers to aid the development of a genetic or sequence map of the C. neoformans genome. The nucleotide sequences and corresponding gene products of the invention can also be used to detect the presence of C. neoformans. Hybridization and antibody-based methods well known in the art can be used to determine the presence and concentration of the nucleotide sequences and corresponding gene products of the invention.

The nucleotide sequences can also be used to make the corresponding gene products which can be used individually or in combination as an immunogen or a subunit vaccine to elicit a protective immune response in animals or subjects at high risk of developing a clinical condition, such as those that are under continual exposure of high levels of C. neoformans organisms or spores.

In yet another embodiment, the invention also encompasses (a) DNA vectors that contain a nucleotide sequence comprising any of the foregoing coding sequences of the target gene and/or their complements (including antisense); (b) DNA expression vectors that contain a nucleotide sequence comprising any of the foregoing coding sequences operably linked with a regulatory element that directs the expression of the coding sequences; and (c) genetically engineered host cells that contain any of the foregoing coding sequences of the target gene operably linked with a regulatory element that directs the expression of the coding sequences in the host cell. Vectors, expression constructs, expression vectors, and genetically engineered host cells containing the coding sequences of homologous target genes of other species (excluding S. cerevisiae, C. albicans, or A. fumigatus) are also contemplated. Also contemplated are genetically engineered host cells containing mutant alleles in homologous target genes of the other species. As used herein, regulatory elements include but are not limited to inducible and non-inducible promoters, enhancers, operators and other elements known to those skilled in the art that drive and regulate expression. Such regulatory elements include but are not limited to the lac system, the trp system, the tet system and other antibiotic-based repression systems (e.g. PIP), the TAC system, the TRC system, the major operator and promoter regions of phage A, the control regions of fd coat protein, and the fungal promoters for 3-phosphoglycerate kinase, acid phosphatase, the yeast mating pheromone responsive promoters (e.g. STE2 and STE3), and promoters isolated from genes involved in carbohydrate metabolism (e.g. GAL promoters including GAL7), phosphate-responsive promoters (e.g. PHO5), or amino acid metabolism (e.g. MET genes). The invention includes fragments of any of the DNA vector sequences disclosed herein.

A variety of techniques can be utilized to further characterize the identified essential genes and virulence genes. First, the nucleotide sequence of the identified genes can be used to reveal homologies to one or more known sequence motifs which can yield information regarding the biological function of the identified gene product. Computer programs well known in the art can be employed to identify such relationships. Second, the sequences of the identified genes can be used, utilizing standard techniques such as in situ hybridization, to place the genes onto chromosome maps and genetic maps which can be correlated with similar maps constructed for another organism, e.g., Saccharomyces cerevisiae, Candida albicans, or Aspergillus fumigatus. The information obtained through such characterizations can suggest relevant methods for using the polynucleotides and polypeptides for discovery of drugs against C. neoformans and other pathogens.

Methods for performing the uses listed above are well known to those skilled in the art. References disclosing such methods include without limitation “Molecular Cloning: A Laboratory Manual,” 2d ed., Cold Spring Harbor Laboratory Press, Sambrook, J., E. F. Fritsch and T. Maniatis eds., 1989, and “Methods in Enzymology: Guide to Molecular Cloning Techniques,” Academic Press, Berger, S. L. and A. R. Kimmel eds., 1987. Basic molecular biology techniques, such as transformation, and gene disruption via homologous recombination can be applied to C. neoformans. Selectable markers are available for genetic manipulation in C. neoformans which include genes conferring antibiotic resistance to nourseothricin acetyltransferase (nat1 gene) of Streptomyces noursei (McDade and Cox (2001) Med. Mycol. 39:151-154); and hygromycin B (CalBioChem, San Diego; plasmid pHYG7-KB1 (Hua et al., (2000) Clin. Diagn Lab Immunol. 7:125-128, which contains the hygromycin gene under the control of C. neoformans H99 actin gene promoter).

5.2.2 Identification of Homologs of C. neoformans Essential Genes

The invention also provides biological and computational methods, and reagents that allow the isolation and identification of genes that are homologous to the identified essential genes of C. neoformans. The identities and uses of such homologous genes are also encompassed by the present invention.

The methods for drug target identification and validation disclosed herein can be directly applied to other haploid pathogenic fungi. Deuteromycetous fungi, i.e. those lacking a sexual cycle and classical genetics represent the majority of human fungal pathogens. In those instances in which a pathogenic fungus is diploid and lacks a haploid life cycle, one allele is knocked out and the second allele is conditionally expressed as disclosed herein.

In the same way medically relevant fungal pathogens are suitable for a rational drug target discovery using the present invention, so too may plant fungal pathogens and animal pathogens be examined to identify novel drug targets for agricultural and veterinary purposes. The quality and yield of many agricultural crops including fruits, nuts, vegetables, rice, soybeans, oats, barley and wheat are significantly reduced by plant fungal pathogens. Examples include the wheat fungal pathogens causing leaf blotch (Septoria tritici, glume blotch (Septoria nodorum), various wheat rusts (Puccinia recondita, Puccinia graminis); powdery mildew (various species), and stem/stock rot (Fusarium spp.). Other particularly destructive examples of plant pathogens include, Phytophthora infestans, the causative agent of the Irish potato famine, the Dutch elm disease causing ascomycetous fungus, Ophiostoma ulmi, the corn smut causing pathogen, Ustilago maydis, the rice-blast-causing pathogen Magnapurtla grisea, Peronospora parasitica (Century et al., Proc Natl Acad Sci U S A 1995 Jul. 3;92(14):6597-601); Cladosporium fulvum (leaf mould pathogen of tomato); Fusarium graminearum, Fusarium culmorum, and Fusarium avenaceum, (wheat, Abramson et al., J Food Prot 2001 August;64(8):1220-5); Alternaria brassicicola (broccoli; Mora et al., Appl Microbiol Biotechnol 2001 April;55(3):306-10); Alternaria tagetica (Gamboa-Angulo et al., J Agric Food Chem 2001 March;49(3):1228-32); the cereal pathogen Bipolaris sorokiniana (Apoga et al., FEMS Microbiol Lett 2001 Apr. 13;197(2):145-50); the rice seedling blast fungus Pyricularia grisea (Lee et al., Mol Plant Microbe Interact 2001 April;14(4):527-35); the anther smut fungus Microbotryum violaceum (Bucheli et al.,: Mol Ecol 2001 February;10(2):285-94); Verticillium longisporum comb. Nov (wilt of oilseed rape, Karapapa et al., Curr Microbiol 2001 March;42(3):217-24); Aspergillus flavus infection of cotton bolls (Shieh et al., Appl Environ Microbiol 1997 September;63(9):3548-52; the eyespot pathogen Tapesia yallundae (Wood et al., FEMS Microbiol Lett 2001 Mar. 15;196(2):183-7); Phytophthora cactorum strain P381 (strawberry leaf necrosis, Orsomando et al., J Biol Chem 2001 Jun. 15;276(24):21578-84); Sclerotinia sclerotiorum, an ubiquitous necrotrophic fungus (sunflowers, Poussereau et al., Microbiology 2001 March;147(Pt 3):717-26); pepper plant/cranberry, anthracnose fungus Colletotrichum gloeosporioides (Kim et al., Mol Plant Microbe Interact 2001 January;14(1):80-5); Nectria haematococca (pea plants, Han et al., Plant J 2001 February;25(3):305-14); Cochliobolus heterostrophus (Monke et al., Mol Gen Genet 1993 October;241(1-2):73-80), Glomerella cingulata (Rodriquez et al., Gene 1987;54(1):73-81) obligate pathogen Bremia lactucae (lettuce downy mildew; Judelson et al., Mol Plant Microbe Interact 1990 July-August;3(4):225-32) Rhynchosporium secalis (Rohe et al., Curr Genet 1996 May;29(6):587-90), Gibberella pulicaris (Fusarium sambucinum), Leptosphaeria maculans (Farman et al., Mol Gen Genet 1992 January;231(2):243-7), Cryphonectria parasitica and Mycosphaerella fijiensis and Mycosphaerella musicola, the causal agents of black and yellow Sigatoka, respectively, and Mycosphaerella eumusae, which causes Septoria leaf spot of banana (banana & plantain, Balint-Kurti et al., FEMS Microbiol Lett 2001 Feb. 5;195(1):9-15). The emerging appearance of fungicidal-resistant plant pathogens and increasing reliance on monoculture practices, clearly indicate a growing need for novel and improved fungicidal compounds. Accordingly, the present invention encompasses identification and validation of drug targets in pathogens and parasites of plants and livestock. Table 2 lists exemplary groups of haploid and diploid fungi of medical, agricultural, or commercial value.

TABLE 2
Exemplary Haploid and Diploid Fungi

| | | |
|---|---|---|
| | Plant Pathogens: | |
| Animal pathogens: | Significance | General Commercial |

Ascomycota

| | | |
|---|---|---|
| Aspergillus fumigatus | Alternaria solanii | Aspergillus niger |
| Alternaria spp | Gaeumannomyces graminis | Schizosaccharomyces pombe |
| Blastomyces dermatidis | Cercospora zeae-maydis | Pichia pastoris |
| Candida spp including | Botrytis cinerea | Hansenula polymorpha |
| Candida dublinensis | Claviceps purpurea | Ashbya gossipii |
| Candida glabrata | Corticum rolfsii | Aspergillus nidulans |
| Candida krusei | Endothia parasitica | Trichoderma reesei |
| Candida lustaniae | Sclerotinia sclerotiorum | Aureobasidium pullulans |
| Candida parapsilopsis | Erysiphe gramini | Yarrowia lipolytica |
| Candida tropicalis | Erysiphe triticii | Candida utilis |
| Coccidioides immitis | Fusarium spp. | Kluveromyces lactis |
| Exophalia dermatiditis | Magnaporthe grisea | |
| Fusarium oxysporum | Plasmopara viticola | |
| Histoplasma capsulatum | Penicillium digitatum | |
| Pneumocystis carinii | Ophiostoma ulmi | |
| | Rhizoctonia species including oryzae | |
| | Septoria species including | |
| | Septoria avenae | |
| | Septoria nodorum | |
| | Septoria passerinii | |
| | Septoria triticii | |
| | Venturia inequalis | |
| | Verticillium dahliae | |
| | Verticillium albo-atrum | |

Basidiomycota

| | | |
|---|---|---|
| Trichosporon beigelii | Puccinia spp including | Agaricus campestris |
| | Puccinia coronata | Phanerochaete chrysosporium |
| | Puccinia graminis | Gloeophyllum traheum |
| | Puccinia recondita | Trametes versicolor |
| | Puccinia striiformis | |
| | Tilletia spp including | |
| | Tilletia caries | |
| | Tilletia controversa | |
| | Tilletia indica | |
| | Tilletia tritici | |
| | Tilletiafoetida | |
| | Ustilago maydis | |
| | Ustilago hordeii | |

Zygomycota

Absidia corymbifera
Mucor rouxii
Rhizomucor pusillus
Rhizopus arrhizus

Thus, in addition to the nucleotide sequences of C. neoformans, described above, homologs or orthologs of these target gene sequences, as can be present in other serotypes and species, can be identified and isolated by molecular biological techniques well known in the art, and without undue experimentation, used in the methods of the invention. Homologous target genes in different serotypes (A, B, C, D, AD) and related strains such as but not limited to Cryptococcus neoformans neoformans, Cryptococcus neoformans grubii and Cryptococcus neoformans gattii are included. Homologous target genes in related species, such as but not limited to, Cryptococcus albidus, Cryptococcus laurentii, Cryptococcus terreus, Cryptococcus uniguttulatus, Cryptococcus luteolus and Cryptococcus gastricus, are included. Moreover, the invention encompasses homologous target genes in Aspergillus flavus, Aspergillus niger, Coccidiodes immitis, Histoplasma capsulatum, Phytophthora infestans, Puccinia seconditii, Pneumocystis carinii, or any species falling within the genera of any of the above species. Other yeasts in the genera of Candida, Saccharomyces, Schizosaccharomyces, Sporobolomyces, Torulopsis, Trichosporon, Tricophyton, Dermatophytes, Microsproum, Wickerhamia, Ashbya, Blastomyces, Candida, Citeromyces, Crebrothecium, Cryptococcus, Debaryomyces, Endomycopsis, Geotrichum, Hansenula, Kloeckera, Kluveromyces, Lipomyces, Pichia, Rhodosporidium, Rhodotorula, and Yarrowia are also contemplated. Also included are homologs of these target gene sequences which can be identified in and isolated from animal fungal pathogens such as Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Alternaria solanii, Botrytis cinerea, Erysiphe graminis, Magnaporthe grisea, Puccinia recodita, Sclerotinia sclerotiorum, Septoria triticii, Tilletia controversa, Ustilago maydis, Venturia inequalis, Verticullium dahliae or any species falling within the genera of any of the above species.

Accordingly, the present invention provides nucleotide sequences that are hybridizable to the polynucleotides of the target genes, and that are of a species other than Saccharomyces cerevisiae, Candida albicans, and Aspergillus fumigatus. Preferably, the species is a fungus, and most preferably a pathogenic species in humans, animals or plants. In one embodiment, the present invention encompasses an isolated nucleic acid comprising a nucleotide sequence that is at least 50% identical to a nucleotide sequence selected from the group consisting of SEQ ID NO.: 1-361, 1001-1361 and 2001-2361. In another embodiment, the present invention encompasses an isolated nucleic acid comprising a nucleotide sequence that hybridizes under medium stringency conditions to a second nucleic acid that consists of a nucleotide sequence selected from the group consisting of SEQ ID NO: 1-361, 1001-1361 and 2001-2361. Where the source of nucleic acid deposited on a gene expression array and the source of the nucleic acid probe being hybridized to the array are from two different species of organisms, the results allow rapid identification of homologous genes in the two species.

In yet another embodiment, the present invention includes an isolated nucleic acid comprising a nucleotide sequence that encodes a polypeptide the amino acid sequence of which is at least 50% identical to an amino acid sequence selected from the group consisting of SEQ ID No.: 3001-3361, as well as the gene product encoded by genomic SEQ ID NO: 1-361 and 1001-1361, as expressed by C. neoformans, wherein the polypeptide is that of a species other than Saccharomyces cerevisiae, Candida albicans, and A. fumigatus. Although the nucleotide sequences and amino acid sequences of homologs or orthologs of such genes in Saccharomyces cerevisiae is mostly published, as well as those homologs or orthologs of such genes in Candida albicans which is available as database version 6 assembled by the Candida albicans Sequencing Project and is accessible by internet at the web sites of Stanford University and University of Minnesota (See http://www-sequence.stanford.edu:8080/ and http://alces.med.umn.edu/Candida.html), uses of many of such homologs or orthologs in S. cerevisae or in C. albicans in drug screening are not known and are thus specifically provided by the invention. To use such nucleotide and/or amino acid sequences of Candida albicans or Saccharomyces cerevisiae, public databases, such as Stanford Genomic Resources (www-genome.stanford.edu), Munich Information Centre for Protein Sequences (www.mips.biochem.mpg.de), or Proteome (www.proteome.com) may be used to identify and retrieve the sequences. In cases where the ortholog or homolog of a C. neoformans gene in C. albicans or Saccharomyces cerevisiae is known, the name of the Saccharomyces cerevisiae and/or Candida albicans gene is indicated in Table I. Orthologs of S. cerevisiae or C. albicans can also be identified by hybridization assays using nucleic acid probes consisting of any one of the nucleotide sequences of SEQ ID NO: 1-361, 1001-1361, or 2001-2361.

The nucleotide sequences of the invention still further include nucleotide sequences that have at least 40%, 45%, 55%, 60%, 65%, 70%, 75%, 80%, 85%, 90%, 95%, 98% or more nucleotide sequence identity to the nucleotide sequences set forth in SEQ ID NO: 1-361, 1001-1361, and 2001-2361. The nucleotide sequences of the invention also include nucleotide sequences that encode polypeptides having at least 25%, 30%, 40%, 50%, 55%, 60%, 65%, 70%, 75%, 80%, 85%, 90%, 95%, 98% or higher amino acid sequence identity or similarity to the amino acid sequences set forth in SEQ ID NO: 3001-3361, as well as the gene product encoded by genomic SEQ ID NO: 1-361 and 1001-1361, as expressed by C. neoformans. Such nucleotide sequences may exclude S. cerevisiae, A. fumigatus and/or C. albicans sequences that are known.

To determine the percent identity of two amino acid sequences or of two nucleotide sequences, the sequences are aligned for optimal comparison purposes (e.g., gaps can be introduced in the sequence of a first amino acid or nucleotide sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul (1990) Proc. Natl. Acad. Sci. U.S.A. 87:2264-2268, modified as in Karlin and Altschul (1993) Proc. Natl. Acad. Sci. U.S.A. 90:5873-5877. Such an algorithm is incorporated into the NBLAST and XBLAST programs of Altschul et al., 1990, J. Mol. Biol. 215:403-0. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

To isolate homologous target genes, the C. neoformans target gene sequence described above can be labeled and used to screen a cDNA library constructed from mRNA obtained from the organism of interest. Hybridization conditions should be of a lower stringency when the cDNA library was derived from an organism different from the type of organism from which the labeled sequence was derived. cDNA screening can also identify clones derived from alternatively spliced transcripts in the same or different species. Alternatively, the labeled fragment can be used to screen a genomic library derived from the organism of interest, again, using appropriately stringent conditions. Low stringency conditions will be well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Spring Harbor Press, N.Y.; and Ausubel et al., 1989, Current Protocols in Molecular Biology, (Green Publishing Associates and Wiley Interscience, N.Y.).

Further, a homologous target gene sequence can be isolated by performing a polymerase chain reaction (PCR) using two degenerate oligonucleotide primer pools designed on the basis of amino acid sequences within the target gene of interest. The template for the reaction can be cDNA obtained by reverse transcription of mRNA prepared from the organism of interest. The PCR product can be subcloned and sequenced to ensure that the amplified sequences represent the sequences of a homologous target gene sequence.

The PCR fragment can then be used to isolate a full length cDNA clone by a variety of methods well known to those of ordinary skill in the art. Alternatively, the labeled fragment can be used to screen a genomic library.

PCR technology can also be utilized to isolate full length cDNA sequences. For example, RNA can be isolated, following standard procedures, from an organism of interest. A reverse transcription reaction can be performed on the RNA using an oligonucleotide primer specific for the most 5′ end of the amplified fragment for the priming of first strand synthesis. The resulting RNA/DNA hybrid can then be “tailed” with guanines using a standard terminal transferase reaction, the hybrid can be digested with RNAase H, and second strand synthesis can then be primed with a poly-C primer. Thus, cDNA sequences upstream of the amplified fragment can easily be isolated. For a review of cloning strategies which can be used, see e.g., Sambrook et al., 1989, Molecular Cloning, A Laboratory Manual, Cold Springs Harbor Press, N.Y.; and Ausubel et al., 1989, Current Protocols in Molecular Biology, (Green Publishing Associates and Wiley Interscience, N.Y.).

Additionally, an expression library can be constructed utilizing DNA isolated from or cDNA synthesized from the organism of interest. In this manner, gene products made by the homologous target gene can be expressed and screened using standard antibody screening techniques in conjunction with antibodies raised against the Candida albicans gene product, as described, below. (For screening techniques, see, for example, Harlow, E. and Lane, eds., 1988, “Antibodies: A Laboratory Manual,” Cold Spring Harbor Press, Cold Spring Harbor). Library clones detected via their reaction with such labeled antibodies can be purified and subjected to sequence analysis by well known methods.

Alternatively, homologous target genes or polypeptides may be identified by searching a database to identify sequences having a desired level of homology to a target gene or polypeptide involved in proliferation, virulence or pathogenicity. A variety of such databases are available to those skilled in the art, including GenBank and GenSeq. In various embodiments, the databases are screened to identify nucleic acids with at least 97%, at least 95%, at least 90%, at least 85%, at least 80%, at least 70%, at least 60%, at least 50%, or at least 40% identity to a target nucleotide sequence, or a portion thereof. In other embodiments, the databases are screened to identify polypeptides having at least 99%, at least 95%, at least 90%, at least 85%, at least 80%, at least 70%, at least 60%, at least 50%, at least 40% or at least 25% identity or similarity to a polypeptide involved in proliferation, virulence or pathogenicity or a portion thereof.

Alternatively, functionally homologous target sequences or polypeptides may be identified by creating mutations that have phenotypes by removing or altering the function of a gene. This can be done for one or all genes in a given fungal species including, for example: Saccharomyces cerevisiae, Candida albicans, and A. fumigatus. Having mutants in the genes of one fungal species offers a method to identify functionally similar genes or related genes (orthologs) in another species, or functionally similar genes in the same species (paralogs), by use of a functional complementation test.

A library of genes or cDNA copies of messenger RNA of genes can be made from a given species, e.g. C. neoformans, and the library cloned into a vector permitting expression (for example, with the C. neoformans, Aspergillus nidulans promoters or Saccharomyces cerevisiae promoters) of the genes in a second species, e.g. Saccharomyces cerevisiae, Aspergillus fumigatus, or Candida albicans. Such a library is referred to as a “heterologous library.” Transformation of the C. neoformans heterologous library into a defined mutant of S. cerevisiae, C. albicans, or A. fumigatus that is functionally deficient with respect to the identified gene, and screening or selecting for a gene in the heterologous library that restores phenotypic function in whole or in part of the mutational defect is said to be “heterologous functional complementation” and in this example, permits identification of gene in C. neoformans that are functionally related to the mutated gene in S. cerevisiae, C. albicans, or A. fumigatus. Inherent in this functional-complementation method, is the ability to restore gene function without the requirement for sequence similarity of nucleic acids or polypeptides; that is, this method permits interspecific identification of genes with conserved biological function, even where sequence similarity comparisons fail to reveal or suggest such conservation.

In those instances in which the gene to be tested is an essential gene, a number of possibilities exist regarding performing heterologous functional complementation tests. The mutation in the essential gene can be a conditional allele, including but not limited to, a temperature-sensitive allele, an allele conditionally expressed from a regulatable promoter, or an allele that has been rendered the mRNA transcript or the encoded gene product conditionally unstable. Alternatively, the strain carrying a mutation in an essential gene can be propagated using a copy of the native gene (a wild type copy of the gene mutated from the same species) on a vector comprising a marker that can be selected against, permitting selection for those strains carrying few or no copies of the vector and the included wild type allele. A strain constructed in this manner is transformed with the heterologous library, and those clones in which a heterologous gene can functionally complement the essential gene mutation, are selected on medium non-permissive for maintenance of the plasmid carrying the wild type gene.

A heterologous functional complementation test is not restricted to the exchange of genetic information between C. neoformans, Aspergillus fumigatus, Candida albicans and Saccharomyces cerevisiae; functional complementation tests can be performed, as described above, using any pair of fungal species. For example, the CRE1 gene of the fungus Sclerotininia sclerotiorum can functionally complement the creAD30 mutant of the CREA gene of Aspergillus nidulans (see Vautard et al. 1999, “The glucose repressor gene CRE 1 from Sclerotininia sclerotiorum is functionally related to CREA from Aspergillus niger but not to the Mig proteins from Saccharomyces cerevisiae,” FEBS Lett. 453: 54-58).

In many of the fungal plant pathogens where homologous target genes are identified, standard genetic manipulation methods that are known to those of skill in the art, e.g., transformation and homologous recombination, are applicable. Non-limiting examples of recombinant gene expression systems include the following: F. oxysporum panC promoter induced by steroidal glycoalkaloid alpha-tomatine (Perez-Espinosa et al.,: Mol Genet Genomics 2001 July;265(5):922-9); Ustilago maydis hsp70-like gene promoter in a high-copy number autonomously replicating expression vector (Keon et al., Antisense Nucleic Acid Drug Dev 1999 February;9(1):101-4); Cochliobolus heterostrophus transient and stable gene expression systems using P1 or GPD1 (glyceraldehyde 3 phosphate dehydrogenase) promoter of C. heterostrophus or GUS or hygromycin B phosphotransferase gene (hph) of E. coli (Monke et al., Mol Gen Genet 1993 October;241(1-2):73-80); Rhynchosporium secalis (barley leaf scald fungus) transformed to hygromycin-B and phleomycin resistance using the hph gene from E. coli and the ble gene from Streptoalloteichus hindustanus under the control of Aspergillus nidulans promoter and terminator sequences, plasmid DNA introduced into fungal protoplasts by PEG/CaCl₂ treatment (Rohe et al., Curr Genet 1996 May;29(6):587-90). Pathogens of banana and plantain (Musa spp.) Mycosphaerella fijiensis and Mycosphaerella musicola, and Mycosphaerella eumusae can be transformed as taught in Balint-Kurti et al., FEMS Microbiol Lett 2001 Feb. 5;195(1):9-15. Gibberella pulicaris (Fusarium sambucinum) a trichothecene-producing plant pathogen can be transformed with three different vectors: cosHyg1, pUCH1, and pDH25, all of which carry hph (encoding hygromycin B phosphotransferase) as the selectable marker (Salch et al., Curr Genet 1993;23(4):343-50). Leptosphaeria maculans, a fungal pathogen of Brassica spp.can be transformed with the vector pAN8-1, encoding phleomycin resistance; protoplasts can be retransformed using the partially homologous vector, pAN7-1 which encodes hygromycin B resistance. Farman et al., Mol Gen Genet 1992 January;231(2):243-7. Cryphonectria parasitica; targeted disruption of enpg-1 of this chestnut blight fungus was accomplished by homologous recombination with a cloned copy of the hph gene of Escherichia coli inserted into exon 1, see Gao et al., Appl Environ Microbiol 1996 June;62(6):1984-90.

Another example, Glomerella cingulata f. sp. phaseoli (Gcp) was transformed using either of two selectable markers: the amdS+gene of Aspergillus nidulans, which encodes acetamidase and permits growth on acetamide as the sole nitrogen source and the hygBR gene of Escherichia coli which permits growth in the presence of the antibiotic Hy. The amdS+ gene functioned in Gcp under control of A. nidulans regulatory signals and hygBR was expressed after fusion to a promoter from Cochliobolus heterostrophus, another filamentous ascomycete. Protoplasts to be transformed were generated with the digestive enzyme complex Novozym 234 and then were exposed to plasmid DNA in the presence of 10 mM CaCl₂ and polyethylene glycol. Transformation occurred by integration of single or multiple copies of either the amdS+ or hygBR plasmid into the fungal genome. (Rodriquez et al., Gene 1987;54(1):73-81); integration vectors for homologous recombination; deletion studies demonstrated that 505 bp (the minimum length of homologous promoter DNA analysed which was still capable of promoter function) was sufficient to target integration events. Homologous integration of the vector resulted in duplication of the gdpA promoter region. (Rikkerink et al., Curr Genet 1994 March;25(3):202-8).

5.2.3 Products Encoded by C. neoformans Essential Genes

The target gene products used and encompassed in the methods and compositions of the present invention include those gene products (e.g., RNA or proteins) that are encoded by the target essential gene sequences as described above, such as, the target gene sequences set forth in SEQ ID NO: 2001-2361. When expressed in an organism which does not use the universal genetic code, protein products of the target genes having the amino acid sequences of SEQ ID NO: 3001-3361, as well as the gene product encoded by genomic SEQ ID NO: 1-361 and 1001-1361, as expressed by C. neoformans, may be encoded by nucleotide sequences that conform to the known codon usage in the organism. One of skill in the art would know the modifications that are necessary to accommodate for a difference in codon usage, e.g., that of Candida albicans.

In addition, however, the methods and compositions of the invention also use and encompass proteins and polypeptides that represent functionally equivalent gene products. Such functionally equivalent gene products include, but are not limited to, natural variants of the polypeptides having an amino acid sequence set forth in SEQ ID NO: 3001-3361, as well as the gene product encoded by genomic SEQ ID NO: 1-361 and 1001-1361, as expressed by C. neoformans.

Such equivalent target gene products can contain, e.g., deletions, additions or substitutions of amino acid residues within the amino acid sequences encoded by the target gene sequences described above, but which result in a silent change, thus producing a functionally equivalent target gene product. Amino acid substitutions can be made on the basis of similarity in polarity, charge, solubility, hydrophobicity, hydrophilicity and/or the amphipathic nature of the residues involved. For example, nonpolar (i.e., hydrophobic) amino acid residues can include alanine (Ala or A), leucine (Leu or L), isoleucine (Ile or I), valine (Val or V), proline (Pro or P), phenylalanine (Phe or F), tryptophan (Trp or W) and methionine (Met or M); polar neutral amino acid residues can include glycine (Gly or G), serine (Ser or S), threonine (Thr or T), cysteine (Cys or C), tyrosine (Tyr or Y), asparagine (Asn or N) and glutamine (Gln or Q); positively charged (i.e., basic) amino acid residues can include arginine (Arg or R), lysine (Lys or K) and histidine (His or H); and negatively charged (i.e., acidic) amino acid residues can include aspartic acid (Asp or D) and glutamic acid (Glu or E).

“Functionally equivalent,” as the term is utilized herein, refers to a polypeptide capable of exhibiting a substantially similar in vivo activity as the C. neoformans target gene product encoded by one or more of the target gene sequences described in Table 2. Alternatively, when utilized as part of assays described hereinbelow, the term “functionally equivalent” can refer to peptides or polypeptides that are capable of interacting with other cellular or extracellular molecules in a manner substantially similar to the way in which the corresponding portion of the target gene product would interact with such other molecules. Preferably, the functionally equivalent target gene products of the invention are also the same size or about the same size as a target gene product encoded by one or more of the target gene sequences.

Peptides and polypeptides corresponding to one or more domains of the target gene products (e.g., signal sequence, TM, ECD, CD, or ligand-binding domains), truncated or deleted target gene products (e.g., polypeptides in which one or more domains of a target gene product are deleted) and fusion target gene proteins (e.g., proteins in which a full length or truncated or deleted target gene product, or a peptide or polypeptide corresponding to one or more domains of a target gene product is fused to an unrelated protein) are also within the scope of the present invention. Such peptides and polypeptides (also referred to as chimeric protein or polypeptides) can be readily designed by those skilled in the art on the basis of the target gene nucleotide and amino acid sequences. Exemplary fusion proteins can include, but are not limited to, epitope tag-fusion proteins which facilitate isolation of the target gene product by affinity chromatography using reagents that binds the epitope. Other exemplary fusion proteins include fusions to any amino acid sequence that allows, e.g., the fusion protein to be anchored to a cell membrane, thereby allowing target gene polypeptides to be exhibited on a cell surface; or fusions to an enzyme (e.g., β-galactosidase encoded by the LAC4 gene of Kluyveronmyces lactis (Leuker et al., 1994, Mol. Gen. Genet., 245:212-217)), to a fluorescent protein (e.g., from Renilla reniformis (Srikantha et al., 1996, J. Bacteriol. 178:121-129), or to a luminescent protein which can provide a marker function. Accordingly, the invention provides a fusion protein comprising a fragment of a first polypeptide fused to a second polypeptide, said fragment of the first polypeptide consisting of at least 6 consecutive residues of an amino acid sequence selected from one of SEQ ID NO: 3001-3361.

Other modifications of the target gene product coding sequences described above can be made to generate polypeptides that are better suited, e.g., for expression, for scale up, etc. in a chosen host cell. For example, cysteine residues can be deleted or substituted with another amino acid in order to eliminate disulfide bridges.

The target gene products of the invention preferably comprise at least as many contiguous amino acid residues as are necessary to represent an epitope fragment (that is, for the gene products to be recognized by an antibody directed to the target gene product). For example, such protein fragments or peptides can comprise at least about 8 contiguous amino acid residues from a full length differentially expressed or pathway gene product. In alternative embodiments, the protein fragments and peptides of the invention can comprise about 6, 10, 20, 30, 40, 50, 60, 70, 80, 90, 100, 150, 200, 250, 300, 350, 400, 450 or more contiguous amino acid residues of a target gene product.

The target gene products used and encompassed in the methods and compositions of the present invention also encompass amino acid sequences encoded by one or more of the above-described target gene sequences of the invention wherein domains often encoded by one or more exons of those sequences, or fragments thereof, have been deleted. The target gene products of the invention can still further comprise post translational modifications, including, but not limited to, glycosylations, acetylations and myristylations.

The target gene products of the invention can be readily produced, e.g., by synthetic techniques or by methods of recombinant DNA technology using techniques that are well known in the art. Thus, methods for preparing the target gene products of the invention are discussed herein. First, the polypeptides and peptides of the invention can be synthesized or prepared by techniques well known in the art. See, for example, Creighton, 1983, Proteins: Structures and Molecular Principles, W. H. Freeman and Co., N.Y., which is incorporated herein by reference in its entirety. Peptides can, for example, be synthesized on a solid support or in solution.

Alternatively, recombinant DNA methods which are well known to those skilled in the art can be used to construct expression vectors containing target gene protein coding sequences such as those set forth in SEQ ID NO: 2001-2361, and appropriate transcriptional/translational control signals. These methods include, for example, in vitro recombinant DNA techniques, synthetic techniques and in vivo recombination/genetic recombination. See, for example, the techniques described in Sambrook et al., 1989, Molecular Cloning: A Laboratory Manual, Cold Spring Harbor Press, Cold Spring Harbor, N. Y., Pla et al., Yeast 12:1677-1702 (1996), which are incorporated by reference herein in their entireties, and Ausubel, 1989, supra. Alternatively, RNA capable of encoding target gene protein sequences can be chemically synthesized using, for example, synthesizers. See, for example, the techniques described in Oligonucleotide Synthesis, 1984, Gait, M. J. ed., IRL Press, Oxford, which is incorporated by reference herein in its entirety.

A variety of host-expression vector systems can be utilized to express the target gene coding sequences of the invention. Such host-expression systems represent vehicles by which the coding sequences of interest can be produced and subsequently purified, but also represent cells which can, when transformed or transfected with the appropriate nucleotide coding sequences, exhibit the target gene protein of the invention in situ. These include but are not limited to microorganisms such as bacteria (e.g., E. coli, B. subtilis) transformed with recombinant bacteriophage DNA, plasmid DNA or cosmid DNA expression vectors containing target gene protein coding sequences; yeast (e.g., Saccharomyces, Schizosaccarhomyces, Neurospora, Aspergillus, Candida, Pichia) transformed with recombinant yeast expression vectors containing the target gene protein coding sequences; insect cell systems infected with recombinant virus expression vectors (e.g., baculovirus) containing the target gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing target gene protein coding sequences; or mammalian cell systems (e.g. COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter). If necessary, the nucleotide sequences of coding regions may be modified according to the codon usage of the host such that the translated product has the correct amino acid sequence.

In bacterial systems, a number of expression vectors can be advantageously selected depending upon the use intended for the target gene protein being expressed. For example, when a large quantity of such a protein is to be produced, for the generation of antibodies or to screen peptide libraries, for example, vectors which direct the expression of high levels of fusion protein products that are readily purified can be desirable. Such vectors include, but are not limited, to the E. coli expression vector pUR278 (Ruther et al., 1983, EMBO J. 2:1791), in which the target gene protein coding sequence can be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

When a target gene is to be expressed in mammalian host cells, a number of viral-based expression systems can be utilized. In cases where an adenovirus is used as an expression vector, the target gene coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing target gene protein in infected hosts, (e.g., See Logan & Shenk, 1984, Proc. Natl. Acad. Sci. USA 81:3655-3659). Specific initiation signals can also be required for efficient translation of inserted target gene coding sequences. These signals include the ATG initiation codon and adjacent sequences. In cases where an entire target gene, including its own initiation codon and adjacent sequences, is inserted into the appropriate expression vector, no additional translational control signals can be needed. However, in cases where only a portion of the target gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner et al., 1987, Methods in Enzymol. 153:516-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used.

For long-term, high-yield production of recombinant proteins, stable expression is preferred. For example, cell lines which stably express the target gene protein can be engineered. Host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the target gene protein. Such engineered cell lines can be particularly useful in screening and evaluation of compounds that affect the endogenous activity of the target gene protein.

A number of selection systems can be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

Alternatively, any fusion protein may be readily purified by utilizing an antibody specific for the fusion protein being expressed. For example, a system described by Janknecht et al. allows for the ready purification of non-denatured fusion proteins expressed in human cells lines (Janknecht et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers. Fusions at the carboxy terminal of the target gene product are also contemplated.

When used as a component in assay systems such as those described herein, the target gene protein can be labeled, either directly or indirectly, to facilitate detection of a complex formed between the target gene protein and a test substance. Any of a variety of suitable labeling systems can be used including but not limited to radioisotopes such as ¹²⁵I; enzyme labeling systems that generate a detectable colorimetric signal or light when exposed to substrate; and fluorescent labels.

Indirect labeling involves the use of a protein, such as a labeled antibody, which specifically binds to either a target gene product. Such antibodies include but are not limited to polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Following expression of the target gene protein encoded by the identified target nucleotide sequence, the protein is purified. Protein purification techniques are well known in the art. Proteins encoded and expressed from identified exogenous nucleotide sequences can be partially purified using precipitation techniques, such as precipitation with polyethylene glycol. Alternatively, epitope tagging of the protein can be used to allow simple one step purification of the protein. In addition, chromatographic methods such as ion-exchange chromatography, gel filtration, use of hydroxyapaptite columns, immobilized reactive dyes, chromatofocusing, and use of high-performance liquid chromatography, may also be used to purify the protein. Electrophoretic methods such as one-dimensional gel electrophoresis, high-resolution two-dimensional polyacrylamide electrophoresis, isoelectric focusing, and others are contemplated as purification methods. Also, affinity chromatographic methods, comprising solid phase bound- antibody, ligand presenting columns and other affinity chromatographic matrices are contemplated as purification methods in the present invention.

In addition, the purified target gene products, fragments thereof, or derivatives thereof may be administered to an individual in a pharmaceutically acceptable carrier to induce an immune response against the protein or polypeptide. Preferably, the immune response is a protective immune response which protects the individual. Methods for determining appropriate dosages of the protein (including use of adjuvants) and pharmaceutically acceptable carriers are familiar to those skilled in the art.

5.2.4 Isolation and Use of Antibodies Recognizing Products Encoded by C. neoformans Essential Genes

Described herein are methods for the production of antibodies capable of specifically recognizing epitopes of one or more of the target gene products described above. Such antibodies can include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to a target gene or gene product, various host animals can be immunized by injection with a target gene protein, or a portion thereof. Such host animals can include but are not limited to rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Accordingly, the invention provides a method of eliciting an immune response in an animal, comprising introducing into the animal an immunogenic composition comprising an isolated polypeptide, the amino acid sequence of which comprises at least 6 consecutive residues of one of SEQ ID NOs: 3001-3361, as well as the gene products, such as splice variants, that are encoded by genomic sequences, SEQ ID NOs: 1-361 and 1001-1361, as expressed by C. neoformans.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, can be immunized by injection with differentially expressed or pathway gene product supplemented with adjuvants as also described above. The antibody titer in the immunized animal can be monitored over time by standard techniques, such as with an enzyme linked immunosorbent assay (ELISA) using immobilized polypeptide. If desired, the antibody molecules can be isolated from the animal (e.g., from the blood) and further purified by well-known techniques, such as protein A chromatography to obtain the IgG fraction.

Monoclonal antibodies, which are homogeneous populations of antibodies to a particular antigen, can be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP J Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

Antibody fragments which recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed (Huse et al., 1989, Science 246:1275-1281) to allow rapid and easy identification of monoclonal Fab fragments with the desired specificity.

Antibodies of the present invention may also be described or specified in terms of their binding affinity to a target gene product. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M,10⁻¹²M, 5×10⁻¹³M, 10⁻¹³M, 5×10⁻¹⁴M, 10⁻¹⁴M, 5×10⁻¹⁵M, or 10⁻¹⁵M.

Antibodies directed against a target gene product or fragment thereof can be used to detect the a target gene product in order to evaluate the abundance and pattern of expression of the polypeptide under various environmental conditions, in different morphological forms (mycelium, yeast, spores) and stages of an organism's life cycle. Antibodies directed against a target gene product or fragment thereof can be used diagnostically to monitor levels of a target gene product in the tissue of an infected host as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ³⁵S or ³H.

Further, antibodies directed against a target gene product or fragment thereof can be used therapeutically to treat an infectious disease by preventing infection, and/or inhibiting growth of the pathogen. Antibodies can also be used to modify a biological activity of a target gene product. Antibodies to gene products related to virulence or pathogenicity can also be used to prevent infection and alleviate one or more symptoms associated with infection by the organism. To facilitate or enhance its therapeutic effect, an antibody (or fragment thereof) may be conjugated to a therapeutic moiety such as a toxin or fungicidal agent. Techniques for conjugating a therapeutic moiety to antibodies are well known, see, e.g., Thorpe et al., “The Preparation And Cytotoxic Properties Of Antibody-Toxin Conjugates,” Immunol. Rev., 62:119-58 (1982).

An antibody with or without a therapeutic moiety conjugated to it can be used as a therapeutic that is administered alone or in combination with chemotherapeutic agents.

5.2.5 Modulation of Essential C. neoformans Gene Expression Using Ribozymes

Ribozymes are enzymatic RNA molecules capable of catalyzing the specific cleavage of RNA (For a review see, for example Rossi, J., 1994, Current Biology 4:469-471). The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins.

Ribozyme molecules designed to catalytically cleave specific target gene mRNA transcripts can also be used to prevent translation of target gene mRNA and expression of target genes. While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target gene mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target gene mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

As in the antisense approach, the ribozymes can be composed of modified oligonucleotides (e.g. for improved stability, targeting, etc.) and should be delivered to cells which express the target gene in vivo. Because ribozymes unlike antisense molecules, are catalytic, a lower intracellular concentration is required for efficiency. Multiple ribozyme molecules directed against different target genes can also be used in combinations, sequentially or simultaneously.

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. These nucleic acid constructs can be administered selectively to the desired cell population via a delivery complex.

Various well-known modifications to the DNA molecules can be introduced as a means of increasing intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

5.2.6 Modulation of Essential C. neoformans Gene Expression Using Antisense Molecules

The use of antisense molecules as inhibitors of gene expression may be a specific, genetically based therapeutic approach (for a review, see Stein, in Ch. 69, Section 5 “Cancer: Principle and Practice of Oncology”, 4th ed., ed. by DeVita et al., J. B. Lippincott, Philadelphia 1993). The present invention provides the therapeutic or prophylactic use of nucleic acids of at least six nucleotides that are antisense to a target essential or virulence gene or a portion thereof in the target organism. An “antisense” target nucleic acid as used herein refers to a nucleic acid capable of hybridizing to a portion of a target gene RNA (preferably mRNA) by virtue of some sequence complementarity. The invention further provides pharmaceutical compositions comprising an effective amount of the antisense nucleic acids of the invention in a pharmaceutically acceptable carrier, as described infra.

In another embodiment, the invention is directed to methods for inhibiting the expression of a target gene in an organism of interest, such as C. neoformans, either in vitro, ex vivo, in vivo, or inside a cell or an organelle of a cell, comprising providing the cell with an effective amount of a composition comprising an antisense nucleic acid of the invention. Multiple antisense polynucleotides hybridizable to different target genes may be used in combinations, sequentially or simultaneously.

In another embodiment, the present invention is directed toward methods for modulating expression of an essential gene which has been identified by the methods described supra, in which an antisense RNA molecule, which inhibits translation of mRNA transcribed from an essential gene, is expressed from a regulatable promoter. In one aspect of this embodiment, the antisense RNA molecule is expressed in a conditional-expression C. neoformans mutant strain. In other aspects of this embodiment, the antisense RNA molecule is expressed in a wild-type strain of Cryptococcus or Aspergillus fumigatus, another haploid or diploid pathogenic organism, including animal fugal pathogens such as Aspergillus niger, Aspergillus flavis, Candida albicans, Candida tropicalis, Candida parapsilopsis, Candida krusei, Cryptococcus neoformans, Coccidioides immilis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydiss, or any species falling within the genera of any of the above species.

The nucleic acid molecule comprising an antisense nucleotide sequence of the invention may be complementary to a coding and/or noncoding region of a target gene mRNA. The antisense molecules will bind to the complementary target gene mRNA transcripts and reduce or prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. One skilled in the art can ascertain a tolerable degree of mismatch by use of standard procedures to determine the melting point of the hybridized complex.

Nucleic acid molecules that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See generally, Wagner, R., 1994, Nature 372:333-335.

Nucleic acid molecules comprising nucleotide sequences complementary to the 5′ untranslated region of the mRNA can include the complement of the AUG start codon. Antisense nucleic acid molecules complementary to mRNA coding regions are less efficient inhibitors of translation but could be used in accordance with the invention. Whether designed to hybridize to the 5′-, 3′- or coding region of target gene mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, at least 50 nucleotides, or at least 200 nucleotides.

Regardless of the choice of target gene sequence, it is preferred that in vitro studies are first performed to quantitate the ability of the antisense molecule to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense molecule can be DNA or RNA or chimeric mixtures or derivatives or modified versions thereof, single-stranded or double-stranded. The antisense molecule can be modified at the base moiety, sugar moiety, or phosphate backbone, for example, to improve stability of the molecule, hybridization, etc. The antisense molecule may include other appended groups such as peptides (e.g., for targeting cell receptors in vivo), hybridization-triggered cleavage agents. (See, e.g., Krol et al., 1988, BioTechniques 6:958-976) or intercalating agents. (See, e.g., Zon, 1988, Pharm. Res. 5:539-549). To this end, the antisense molecule may be conjugated to another molecule, e.g., a peptide, hybridization triggered cross-linking agent, transport agent, hybridization-triggered cleavage agent, etc.

The antisense molecule may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense molecule may also comprise at least one modified sugar moiety selected from the group including but not limited to arabinose, 2-fluoroarabinose, xylulose, and hexose.

In yet another embodiment, the antisense molecule comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense molecule is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Antisense molecules of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the coding region of a target gene could be used, those complementary to the transcribed untranslated region are also preferred.

Pharmaceutical compositions of the invention comprising an effective amount of an antisense nucleic acid in a pharmaceutically acceptable carrier, can be administered to a subject infected with the pathogen of interest.

The amount of antisense nucleic acid which will be effective in the treatment of a particular disease caused by the pathogen will depend on the site of the infection or condition, and can be determined by standard techniques. Where possible, it is desirable to determine the antisense cytotoxicity of the pathogen to be treated in vitro, and then in useful animal model systems prior to testing and use in humans.

A number of methods have been developed for delivering antisense DNA or RNA to cells; e.g., antisense molecules can be injected directly into the tissue site in which the pathogens are residing, or modified antisense molecules, designed to target the desired cells (e.g., antisense molecule linked to peptides or antibodies that specifically bind receptors or antigens expressed on the pathogen's cell surface) can be administered systemically. Antisense molecules can be delivered to the desired cell population via a delivery complex. In a specific embodiment, pharmaceutical compositions comprising antisense nucleic acids of the target genes are administered via biopolymers (e.g., poly-β-1→4-N-acetylglucosamine polysaccharide), liposomes, microparticles, or microcapsules. In various embodiments of the invention, it may be useful to use such compositions to achieve sustained release of the antisense nucleic acids. In a specific embodiment, it may be desirable to utilize liposomes targeted via antibodies to specific identifiable pathogen antigens (Leonetti et al., 1990, Proc. Natl. Acad. Sci. U.S.A. 87:2448-2451; Renneisen et al., 1990, J. Biol. Chem. 265:16337-16342).

5.2.7 Construction of C. neoformans Strains Carrying Mutant Essential Genes

In one embodiment of the present invention, each of the essential genes of the invention is placed under the control of the heterologous promoter, the activity of which is regulatable. Where the gene is essential, elimination of expression of that gene will be lethal or severely crippling for growth. Therefore, in the present invention, a heterologous promoter is used to provide a range of levels of expression of a target. Depending on the conditions, the gene may be under-expressed, over-expressed, or expressed at a level comparable to that observed when the target gene is linked to its native promoter. A heterologous promoter is a promoter from a different gene from the same pathogenic organism, or it can be a promoter from a different species.

Suitable promoters for use in C. neoformans include, but are not limited to, the actin promoter (Nelson et al., (2001) Genetics 157(3):935-947); constitutive; the gpd promoter (Varma and Kwon-Chung (1999) Gene 232:155-163); and the GAL7 gene promoter; modestly regulated by carbon source in a serotype A strain of C. neoformans (Del Poeta et al., (1999) Genetics 152:167-178). Expression of C. neoformans GAL7 mRNA was first observed within 2.5 min of induction and fully induced by 30 min. The gene was completely repressed in the presence of glucose. The GAL7 promoter was isolated and used to construct a promoter cassette. Two genes were tested in this cassette for galactose regulation by creating GAL7 promoter fusions with their coding regions. MF alpha, which encodes a pheromone, was found to produce filaments only in transformants that were induced by galactose. The level of enzyme activity was at least 500-fold greater for cells grown in galactose than for cells grown in glucose (Wickes and Edman (1995) Mol. Microbiol. 16:1099-109). These promoters are useful for the in vitro expression of recombinant proteins in C. neoformans or for expression of selectable markers and reporters, analysis of essentiality of a given gene product in vitro. However, for in vivo validation experiments, promoters that direct a broad range of expression levels are preferred; and promoters that have a very low basal level of expression in the repressed state are most desired. Generally, use of promoters that are involved in carbon source utilization is less preferred for experiments in animal models due to (i) metabolism of the inducer/repressor by the cells, and (ii) difficulty in sustaining sufficiently high level of inducer/repressor in the blood of the animal.

The process of replacing the cognate promoter with a heterologous promoter can be repeated for a desired subset of genes such that a collection of conditional-expression mutant C. neoformans strains is generated wherein each strain comprises a different, conditionally-expressed gene. By repeating this process for every gene, a substantially complete set of C. neoformans mutant haploid strains representing the entire genome of the pathogenic fungus can be obtained. Thus, the present invention provides a method of assembling a collection of mutant C. neoformans cells, each of which comprises a different gene under the control of a heterologous promoter. A preferred embodiment for the construction of conditional-expression mutants of C. neoformans strains, uses the following, non-limiting method.

In one embodiment of the present invention, essential genes of C. neoformans are conditionally expressed by replacing the native promoter with a conditional-expression promoter, such as the tetracycline-regulatable promoter system that is developed initially for Saccharomyces cerevisiae but which can be modified for use in C. neoformans (See Gari et al., 1997, Yeast 13:837-848; and Nagahashi et al., 1997, Mol. Gen. Genet. 255:372-375).

In this embodiment, conditional expression is achieved by first constructing a transactivation fusion protein comprising the E. coli TetR tetracycline repressor domain or DNA binding domain (amino acids 1-207) fused to the transcription activation domain of Saccharomyces cerevisiae GAL4 (amino acids 785-881) or HAP4 (amino acids 424-554). The nucleotide sequences encoding the transactivation fusion proteins of E. coli TetR (amino acids 1-207) plus Saccharomyces cerevisiae GAL4 (amino acids 785-881), and of E. coli TetR (amino acids 1-207) plus Saccharomyces cerevisiae HAP4 (amino acids 424-554), is encompassed by the present invention. Other promoters that can be used include the actin promoter (Nelson et al., (2001) Genetics 157(3):935-947); the gpd promoter (Varma and Kwon-Chung (1999) Gene 232:155-163); and the GAL7 gene promoter; of C. neoformans (Del Poeta et al., (1999) Genetics 152:167-178). Accordingly, the invention provides C. neoformans cells that comprise a nucleotide sequence encoding a transactivation fusion protein expressible in the cells, wherein the transactivation fusion protein comprises a DNA binding domain and a transcription activation domain.

Expression of the transactivation fusion protein in C. neoformans is achieved by providing, in one non-limiting example, an Aspergillus niger glucoamylase promoter, PGLA A. However, it will be appreciated that any regulatory regions, promoters and terminators, that are functional in C. neoformans can be used to express the fusion protein. Thus, a nucleic acid molecule comprising a promoter functional in C. neoformans, the coding region of a transactivation fusion protein, and a terminator functional in C. neoformans, are encompassed by the present invention. Such a nucleic acid molecule can be a plasmid, a cosmid, a transposon, or a mobile genetic element. In a preferred embodiment, the TetR-Gal4 or TetR-Hap4 transactivators are stably integrated into a C. neoformans strain, by using a suitable auxotrophic marker (e.g., ade2) for selection of the desired integrant.

PCR amplification of a dominant selectable marker so as to include about 65 bp of flanking sequence identical to the sequence 5′ and 3′ of the C. neoformans gene to be disrupted, allows construction of a gene disruption cassette for any given C. neoformans gene. In this embodiment, the promoter replacement fragment comprises a nucleotide sequence encoding a heterologous promoter that comprises at least one copy of a nucleotide sequence recognized by the DNA binding domain of the transactivation fusion protein, whereby binding of the transactivation fusion protein to the heterologous promoter increases transcription from that promoter. The heterologous tetracycline promoters initially developed for Saccharomyces cerevisiae gene expression contains a variable number of copies of the tetracycline operator sequence, i.e., 2, 4, or 7 copies. The tetracycline promoter is subcloned adjacent to, e.g., a ADE2 or a URA5 selectable marker, in the orientation favoring tetracycline promoter-dependent regulation when placed immediately upstream the open reading frame of the target gene. PCR amplification of the URA5-Tet or ADE2-Tet promoter cassette incorporates approximately 65 bp of flanking sequence homologous to the regulatory region to be replaced, that is, the region from around nucleotide positions -200 and -1 (relative to the start codon) of the target gene, thereby producing a conditional-expression promoter replacement fragment for transformation. When transformed into a C. neoformans homologous recombination between the promoter replacement fragment and the upstream regulatory region of the target gene generates a strain in which the wild type regulatory region is replaced by the conditionally regulated tetracycline promoter. Transformants are selected as uracil prototrophs (URA5-TET) or adenine prototrophs (ADE2-TET) and verified by Southern blot and PCR analysis.

An example of targeted gene disruption in C. neoformans using the phosphoribosylaminoimidazole carboxylase (ADE2) gene which is required for purine biosynthesis, as a selectable marker is provided in Davidson et al., 2000, Fungal Genet. Biol. 29:38-48, and Sudarshan et al., 1999, Fungal Genet. Biol. 27:36-48; which are incorporated herein by reference in its entirety. For serotype A strain H99, ade2 mutations in strains M001 and M049 can serve as recipients for target gene disruptions using the ADE2 gene. Adenine prototrophic transformants are selected on minimal medium containing 1 M sorbitol and lacking exogenous adenine. The C. neoformans URA5 gene encodes orotidine monophosphate pyrophosphorylase (OMPPase) which complements the pyrE mutation in E. coli and which can transform C. neoformans ura5 mutants to uracil prototrophy. It has been shown that all the C. neoformans uracil auxotrophs obtained with 5-FOA selection were ura5 mutants. Methods of use of this selective marker for transformation is described in Edman et al., 1990, Mol. Cell Biol. 10:4538-4544, which is incorporated by reference herein in its entirety.

In this particular embodiment, the promoter is induced in the absence of tetracycline, and repressed by the presence of tetracycline. Analogs of tetracycline, including but not limited to chlortetracycline, demeclocycline, doxycycline, meclocycline, methocycline, minocycline hydrochloride, anhydrotetracycline, and oxytetracycline, can also be used to repress the expression of the conditional-expression mutant of the C. neoformans target gene.

The present invention also encompasses the use of alternative variants of the tetracycline promoter system, based upon a mutated tetracycline repressor (tetR) molecule, designated tetR′, which is activated (i.e. binds to its cognate operator sequence) by binding of the antibiotic effector molecule to promote expression, and is repressed (i.e. does not bind to the operator sequence) in the absence of the antibiotic effectors, when the tetR′ is used instead of, or in addition to, the wild-type tetR. For example, analysis of the essentiality of a C. neoformans gene could be performed using tetR′ instead of tetR in cases where repression is desired under conditions which lack the presence of tetracycline, such as shut off of a gene participating in drug transport (e.g. C. neoformans homologs of the CaCDR1, CaPDR5, or CaMDR1 genes of Candida albicans). Also, the present methods could be adapted to incorporate both the tetR and tetR′ molecules in a dual activator/repressor system where tetR is fused to an activator domain and tetR′ is fused to a general repressor (e.g. the C. neoformans homologs of the Candida albicans genes CaSsr6 and CaTup1) to enhance or further repress expression in the presence of the antibiotic effector molecules (Belli et al., 1998, Nucl Acid Res 26:942-947 which is incorporated herein by reference). These methods of providing conditional expression are also contemplated. By repeating this process whereby the wild type promoter for an C. neoformans gene is replaced with a conditionally-expressed heterologous promoter, for a preferred subset of genes of C. neoformans, or its entire genome, a collection or a complete set of conditional-expression mutant strains of C. neoformans is obtained.

In another embodiment of the invention, the method is also applied to other haploid pathogenic fungi by modifying the target gene via recombination of the allele with a promoter replacement fragment comprising a nucleotide sequence encoding a heterologous promoter, such that the expression of the gene is conditionally regulated by the heterologous promoter. A preferred subset of genes comprises genes that share substantial nucleotide sequence homology with target genes of other organisms, e.g., Aspergillus funigatus, Candida albicans and Saccharomyces cerevisiae. For example, this method of the invention may be applied to other haploid fungal pathogens including, but not limited to, animal fugal pathogens such as Aspergillus niger, Aspergillus flavis, Candida glabrata, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species.

The means to achieve conditional expression are not restricted to the tetracycline promoter system and can be performed using other conditional promoters. Such conditional promoter may, for example, be regulated by a repressor which repress transcription from the promoter under particular condition or by a transactivator which increases transcription from the promoter, such as, when in the presence of an inducer. Alternative promoters that could functionally replace the tetracycline promoter include but are not limited to other antibiotic-based regulatable promoter systems (e.g., pristinamycin-induced promoter or PIP) as well as the C. neoformans homologs of Candida albicans conditionally-regulated promoters such as MET25, MAL2, PHO5, GAL1, 10, STE2, or STE3.

In other embodiments of the invention, conditional expression is achieved by means other than the reliance of conditional promoters. For example, conditional expression could be achieved by the replacement of the wild type allele with temperature sensitive alleles derived in vitro, and their phenotype would then be analyzed at the nonpermissive temperature. In a related approach, insertion of a ubiquitination signal into the a gene to destabilize the encoded gene product during activation conditions can be adopted to examine phenotypic effects resulting from gene inactivation. Collectively, these examples demonstrate the manner in which C. neoformans genes can be disrupted and conditionally regulated using the methods disclosed herein.

In an alternative embodiment of the present invention, a constitutive promoter regulated by an excisable transactivator can be used. The promoter is placed upstream to a target gene to repress expression to the basal level characteristic of the promoter. For example, in a fungal cell, a heterologous promoter containing lexA operator elements may be used in combination with a fusion protein composed of the lexA DNA binding domain and any transcriptional activator domain (e.g. GAL4, HAP4, VP16) to provide constitutive expression of a target gene. Counterselection mediated by 5-FOA can be used to select those cells which have excised the gene encoding the fusion protein. This procedure enables an examination of the phenotype associated with repression of the target gene to the basal level of expression provided by the lexA heterologous promoter in the absence of a functional transcription activator. The conditional-expression C. neoformans mutant strains generated by this approach can be used for drug target validation as described in detail in the sections below. In this system, the low basal level expression associated with the heterologous promoter is critical. Thus, it is preferable that the basal level of expression of the promoter is low to make this alternative shut-off system more useful for target validation.

Alternatively, conditional expression of a target gene can be achieved without the use of a transactivator containing a DNA binding, transcriptional activator domain. A cassette could be assembled to contain a heterologous constitutive promoter downstream of, for example, the URA5 selectable marker, which is flanked with a direct repeat containing homologous sequences to the 5′ portion of the target gene. Additional homologous sequences upstream of the target, when added to this cassette would facilitate homologous recombination and replacement of the native promoter with the above-described heterologous promoter cassette immediately upstream of the start codon of the target gene or open reading frame. Conditional expression is achieved by selecting uracil prototrophic strains, by using the appropriate media, which have integrated the heterologous constitutive promoter and URA5 marker and examining the growth of the resulting strain versus a wild type strain grown under identical conditions. Subsequent selection of 5-FOA resistant strains provides isolates which have lost the URA5 marker and heterologous, constitutive promoter, allowing a comparison between the growth of the resulting strain lacking a promoter for expression of the target gene and the growth of a wild type strain cultured under identical conditions.

5.2.8 Construction of Mutant Diploid Strains

Environmental and clinical isolates of C. neoformans are predominantly haploid. Stable diploid strains can be isolated by genetic crosses of congenic genetically marked strains. These diploid strains are self-fertile and grow as yeast cells as 37° C. but spontaneously filament and spoulate when cultured at 24° C. These diploid strains are heterozygous at the MAT locus (MATα/MATa) and can be transformed and sporulated with meiotic segregation of an integrated marker. For a detailed description of the characterization and techniques for handling such diploid strains, see Sia et al., 2000, Fungal Genet. Biol., 29:153-163; and Lengeler et al., 2001, Infection Immunity, 69:115-122, which are both incorporated herein by reference in its entirety.

According to the invention, diploid forms of mutant Cryptococcus neoformans are also encompassed. In these diploid forms, only one allele of a gene is eliminated, while the second allele is placed under the control of the heterologous promoter, the activity of which is regulatable as described in the previous section. Where the gene is essential, elimination of both alleles will be lethal or severely crippling for growth. Therefore, in the present invention, a heterologous promoter is used to provide a range of levels of expression of the second allele. Depending on the conditions, the second allele can be non-expressing, underexpressing, overexpressing, or expressing at a normal level relative to that when the allele is linked to its native promoter.

Precise replacement of a target gene is facilitated by using a gene disruption cassette comprising a selectable marker, preferably a dominant selectable marker, that is expressible in the strain of interest. The availability of two distinct dominant selectable markers allows the gene replacement process to be engineered at both alleles of the target gene, without the required counterselection step inherent in existing methods.

In particular, the present invention encompasses a method for constructing a strain of diploid pathogenic fungal cells, in which both alleles of a gene are modified, the method comprising the steps of (a) modifying a first allele of a gene in diploid pathogenic fungal cells by recombination using a gene disruption cassette comprising a nucleotide sequence encoding a selectable marker that is expressible in the cells, thereby providing heterozygous pathogenic fungal cells in which the first allele of the gene is inactivated; and (b) modifying the second allele of the gene in the heterozygous diploid pathogenic fungal cells by recombination with a promoter replacement fragment comprising a heterologous promoter, such that the expression of the second allele of the gene is regulated by the heterologous promoter.

The process can be repeated for a desired subset of the genes such that a collection of C. neoformans diploid strains is generated wherein each strain comprises a modified allelic pair of a different gene. By repeating this process for every gene in a pathogenic fungus, a complete set of C. neoformans diploid strains representing the entire genome of the pathogenic fungus can be obtained. Thus, the present invention provides a method of assembling a collection of diploid pathogenic fungal cells, each of which comprises the modified alleles of a different gene. The method comprises repeating the steps of modifying pairs of alleles a plurality of times, wherein a different pair of gene alleles is modified with each repetition, thereby providing the collection of diploid pathogenic fungal cells each comprising the modified alleles of a different gene.

Various embodiments for the construction of such C. neoformans diploid strains, uses the following two-step method. Several art-known methods are available to create a heterozygote mutant. Auxotrophic markers, such as but not limited to the Cryptococcus neoformans equivalents of CaURA3, CaHIS3, CaLEU2, or CaTRP1, could be used for gene disruption if desired. However, the preferred method of heterozygote construction in diploid fungi employs a genetically modified dominant selectable marker. C. neoformans is sensitive to the nucleoside-like antibiotic streptothricin and the presence of the drug resistance gene of Streptomyces noursei gene within C. neoformans allows acetylation of the drug rendering it nontoxic and permitting the strain to grow in the presence of streptothricin.

PCR amplification of either dominant selectable marker so as to include about 65 bp of flanking sequence identical to the sequence 5′ and 3′ of the C. neoformans gene to be disrupted, allows construction of a gene disruption cassette for any given C. neoformans gene. By employing the method of Baudin et al. (1993, Nucleic Acids Research 21:3329-30), a gene disruption event can be obtained following transformation of a C. neoformans strain with the PCR-amplified gene disruption cassette and selection for drug resistant transformants that have precisely replaced the wild type gene with the dominant selectable marker. Such mutant strains can be selected for growth in the presence of a drug, such as but not limited to streptothricin. The resulting gene disruptions are generally heterozygous in the diploid C. neoformans, with one copy of the allelic pair on one homologous chromosome disrupted, and the other allele on the other homologous chromosome remaining as a wild type allele as found in the initial parental strain. The disrupted allele is non-functional, and expression from this allele of the gene is nil. By repeating this process for all the genes in the genome of an organism, a set of gene disruptions can be obtained for every gene in the organism. The method can also be applied to a desired subset of genes.

The conditional expression system used in this embodiment of the invention comprises a regulatable promoter and a means for regulating promoter activity. Conditional expression of the remaining wild type allele in a heterozygote constructed as set forth in Section 5.2.7 is achieved by replacing its promoter with a tetracycline-regulatable promoter system that is developed initially for S. cerevisiae. See Gari et al., 1997, Yeast 13:837-848; and Nagahashi et al., 1997, Mol. Gen. Genet. 255:372-375. Targeted gene disruption in C. neoformans using the phosphoribosylaminoimidazole carboxylase (ADE2) gene as a selectable marker can be used as described above. Strains of C. neoformans that are deficient in ADE2 are used in these methods (e.g., JEC 170, JSC50 and JEC156).

In a preferred embodiment of this method, performing the gene disruption first enables heterozygous strains to be constructed and separately collected as a heterozygote strain collection during the process of drug target validation. Such a C. neoformas heterozygote strain collection enables drug screening approaches based on haploinsufficiency for validated targets within the collection. As used herein, the term “haploinsufficiency” refers to the phenomenon whereby heterozygous strains for a given gene express approximately half the normal diploid level of a particular gene product. Consequently, these strains provide constructions having a diminished level of the encoded gene product, and they may be used directly in screens for antifungal compounds. Here differential sensitivity of a diploid parent, as compared with its heterozygous derivative, will indicate that a drug is active against the encoded gene product.

It is clear to those skilled in the art that the order of allele modification followed in this embodiment of the invention is not critical, and that it is feasible to perform these steps in a different order such that the conditional-expressing allele is constructed first and the disruption of the remaining wild type gene allele be performed subsequently. However, where the promoter replacement step is carried out first, care should be taken to delete sequences homologous to those employed in the gene disruption step.

### 5.3 Identification and Validation of Essential Genes

5.3.1 Target Genes

Target discovery has traditionally been a costly, time-consuming process, in which newly-identified genes and gene products have been individually analyzed as potentially-suitable drug targets. DNA sequence analysis of entire genomes has markedly accelerated the gene discovery process. Consequently, new methods and tools are required to analyze this information, first to identify all of the genes of the organism, and then, to discern which genes encode products that will be suitable targets for the discovery of effective, non-toxic drugs. Gene discovery through sequence analysis alone does not validate either known or novel genes as drug targets. Elucidation of the function of a gene from the underlying and a determination of whether or not that gene is essential still present substantial obstacles to the identification of appropriate drug targets.

As noted above, Cryptococcus neoformans is a major fungal pathogen of immunocompromised humans. An absence of identified specific, sensitive, and unique drug targets in this organism has hampered the development of effective, non-toxic compounds for clinical use. The recent extensive DNA sequence analysis of the Cryptococcus neoformans genome is rejuvenating efforts to identify new antifungal drug targets. Nevertheless, two primary obstacles to the exploitation of this information for the development of useful drug targets remain: the paucity of suitable markers for genetic manipulations in Cryptococcus neoformans and the inherent difficulty in establishing whether a specific gene encodes an essential product.

5.3.2 Validation of Genes Encoding Drug Targets

Target gene validation refers to the process by which a gene product is identified as suitable for use in screening methods or assays in order to find modulators of the function or structure of that gene product. Criteria used for validation of a gene product as a target for drug screening, however, may be varied depending on the desired mode of action that the compounds sought will have, as well as the host to be protected.

In one aspect of the present invention, conditional-expression C. neoformans mutant strains having modified essential genes can be used directly for drug screening. In another aspect, the initial set of essential genes is further characterized using, for example, nucleotide sequence comparisons, to identify a subset of essential genes which include only those genes specific to fungi—that is, a subset of genes encoding essential genes products which do not have homologs in a host of the pathogen, such as humans. Modulators, and preferably inhibitors, of such a subset of genes in a fungal pathogen of humans would be predicted to be much less likely to have toxic side effects when used to treat humans.

Similarly, other subsets of the larger essential gene set could be defined to include only those conditional-expression C. neoformans mutant strains carrying modified genes that do not have a homologous sequence in one or more host (e.g., mammalian) species to allow the detection of compounds expected to be used in veterinary applications. In addition, using other homology criteria, a subset of conditional-expression C. neoformans mutant strains is identified and used for the detection of anti-fungal compounds active against agricultural pathogens, inhibiting targets that do not have homologs in the crop to be protected.

Current C. neoformans gene disruption strategies identify nonessential genes and permit the inference that other genes are essential, based on a failure to generate a null mutant. The null phenotype of a drug target predicts the absolute efficaciousness of the “perfect” drug acting on this target, such as a cidal (cell death) versus static (inhibitory growth) null terminal phenotype for a particular drug target.

For example, in Candida albicans, gene disruption of CaERG11, the drug target of fluconazole, is presumed to be essential based on the failure to construct a homozygous CaERG11 deletion strain using the URA blaster method. However, direct evaluation of its null phenotype being cidal or static could not be performed in the pathogen, and only after the discovery of fluconazole was it possible to biochemically determine both the drug, and presumably the drug target to be static rather than as cidal. Despite the success fluconazole enjoys in the marketplace, its fungistatic mode of action contributes to its primary limitation, i.e., drug resistance after prolonged treatment. Therefore, for the first time, the ability to identify and evaluate cidal null phenotypes for validated drug targets within the pathogen as provided by the invention, now enables directed strategies to identifying antifungal drugs that specifically display a fungicidal mode of action.

Using a single conditional-expression C. neoformans mutant strain or a desired collection of conditional-expression C. neoformans mutant strains comprising essential genes, one or more target genes can be directly evaluated as displaying either a cidal or static null phenotype. This is determined by first incubating conditional-expression C. neoformans mutant strains under repressing conditions for the conditional expression of the modified gene for varying lengths of time in liquid culture, and measuring the percentage of viable cells following plating a defined number of cells onto growth conditions which relieve repression. The percentage of viable cells that remain after return to non-repressing conditions reflects either a cidal (low percent survival) or static (high percent survival) phenotype. Alternatively, vital dyes such as methylene blue or propidium iodide could be used to quantify percent viability of cells for a particular strain under repressing versus inducing conditions. As known fungicidal drug targets are included in the conditional-expression C. neoformans mutant strains strain collection, direct comparisons can be made between this standard fungicidal drug target and novel targets comprising the drug target set. In this way each member of the target set can be immediately ranked and prioritized against an industry standard cidal drug target to select appropriate drug targets and screening assays for the identification of the most rapid-acting cidal compounds. Accordingly, in preferred embodiments, mutations of the essential genes of the invention confer to the cells a rapid cidal phenotype.

### 5.4 Screening Assays

The following assays are designed to identify compounds that bind to target gene products, bind to other cellular proteins that interact with the target gene product, and to compounds that interfere with the interaction of the target gene product with other cellular proteins. Compounds identified via such methods can include compounds which modulate the activity of a polypeptide encoded by a target gene of the invention (that is, increase or decrease its activity, relative to activity observed in the absence of the compound). Alternatively, compounds identified via such methods can include compounds which modulate the expression of the polynucleotide (that is, increase or decrease expression relative to expression levels observed in the absence of the compound), or increase or decrease the stability of the expressed product encoded by that polynucleotide. Compounds, such as compounds identified via the methods of the invention, can be tested using standard assays well known to those of skill in the art for their ability to modulate activity/expression.

Accordingly, the present invention provides a method for identifying an antimycotic compound comprising screening a plurality of compounds to identify a compound that modulates the activity or level of a gene product, said gene product being encoded by a nucleotide sequence selected from the group consisting of SEQ ID NOs: 2001-2361, as well as the gene product encoded by genomic SEQ ID NOs: 1-361 and 1001-1361, as expressed by C. neoformans, or a nucleotide sequence that is naturally occurring in Saccharomyces cerevisiae Candida albicans, Aspergillus fumigatus and that is the ortholog of a gene having a nucleotide sequence selected from the group consisting of SEQ ID NOs: 2001-2361.

5.4.1 In Vitro Screening Assays

In vitro screening assays are designed to identify compounds capable of binding the target gene products of the invention. Compounds identified in this manner are useful, for example, in modulating the activity of wild type and/or mutant target gene products, are useful in elucidating the biological function of target gene products, are utilized in screens for identifying other compounds that disrupt normal target gene product interactions, or are useful themselves for the disruption of such interactions.

The principle of the assays used to identify compounds that bind to the target gene product involves preparing a reaction mixture comprising the target gene product and the test compound under conditions and for a time sufficient to allow the two components to interact and bind, thus forming a complex which is removed and/or detected within the reaction mixture. These assays are conducted in a variety of ways. For example, any method that detects an altered physical property (e.g., size, mobility, etc.) of a target gene product of the invention complexed to a test compound from an unbound target gene product of the invention can be used in the methods of the invention, including, but not limited to, electrophoresis, size exclusion chromatography, and mass spectrometry. Other methods to detect binding between polypeptide molecules of the invention and test compounds directly can also be used, including, but not limited to, affinity chromatography, scintillation proximity assay, nuclear magnetic resonance spectroscopy, and fluorescence resonance energy transfer.

In a first embodiment, electrophoresis is used to identify test compounds capable of binding a polypeptide of the invention. In general, a polypeptide molecule of the invention bound to a test compound is larger than an unbound polypeptide molecule of the invention. Electrophoretic separation based on size allows for determination of such a change in size. Any method of electrophoretic separation, including but not limited to, denaturing and non-denaturing polyacrylamide gel electrophoresis, urea gel electrophoresis, gel filtration, pulsed field gel electrophoresis, two dimensional gel electrophoresis, continuous flow electrophoresis, zone electrophoresis, agarose gel electrophoresis, and capillary electrophoresis can be used. In a preferred embodiment, an automated electrophoretic system comprising a capillary cartridge having a plurality of capillary tubes is used for high-throughput screening of test compounds capable of binding a target gene product of the invention. Such an apparatus for performing automated capillary gel electrophoresis is disclosed in U.S. Pat. Nos. 5,885,430; 5,916,428; 6,027,627; and 6,063,251. In another preferred embodiment, an automated electrophoretic system can comprise a chip-based microfluidic system for high-speed electrophoretic analysis See, for example, U.S. Pat. No. 5,699,157.

In a second embodiment, size exclusion chromatography is used to identify test compounds capable of binding polypeptide molecules of the invention. Size-exclusion chromatography separates molecules based on their size and uses gel-based media comprised of beads with specific size distributions. When applied to a column, this media settles into a tightly packed matrix and forms a complex array of pores. Separation is accomplished by the inclusion or exclusion of molecules by these pores based on molecular size. Small molecules are included into the pores and, consequently, their migration through the matrix is retarded due to the added distance they must travel before elution. Large molecules are excluded from the pores and migrate with the void volume when applied to the matrix. In the present invention, a target gene product of the invention bound to a test compound will be larger, and thus elute faster from the size exclusion column, than an unbound polypeptide molecule.

In a third embodiment, mass spectrometry is used to identify test compounds capable of binding polypeptides of the invention. An automated method for analyzing mass spectrometer data which can analyze complex mixtures containing many thousands of components and can correct for background noise, multiply charged peaks and atomic isotope peaks is described in U.S. Pat. No. 6,147,344.

In another embodiment, affinity chromatography is used to identify test compounds capable of binding target gene products of the invention. To accomplish this, a target gene product of the invention is labeled with an affinity tag (e.g., GST, HA, myc, streptavidin, biotin) such that the polypeptide molecule of the invention can attach to a solid support through interaction with the affinity tag and solid support medium. The tagged polypeptide of the invention is contacted with a test compound either while free in solution or while bound to a solid support. The solid support an comprise, but is not limited to, cross-linked agarose beads that are coupled with a ligand for the affinity tag. Alternatively, the solid support may be a glass, silicon, metal, or carbon, plastic (polystyrene, polypropylene) surface with or without a self-assembled monolayer either with a covalently attached ligand for the affinity tag, or with inherent affinity for the tag on the polypeptide molecule of the invention.

Once the complex between the target gene product of the invention and test compound has reached equilibrium and has been captured, one skilled in the art will appreciate that the retention of bound compounds and removal of unbound compounds is facilitated by washing the solid support with large excesses of binding reaction buffer. Furthermore, retention of high affinity compounds and removal of low affinity compounds can be accomplished by a number of means that increase the stringency of washing; these means include, but are not limited to, increasing the number and duration of washes, raising the salt concentration of the wash buffer, addition of detergent or surfactant to the wash buffer, and addition of non-specific competitor to the wash buffer.

Following the removal of unbound compounds, bound compounds with high affinity for the immobilized polypeptide molecule of the invention can be eluted and analyzed. The elution of test compounds can be accomplished by any means that break the non-covalent interactions between the polypeptide of the invention and test compound. Means for elution include, but are not limited to, changing the pH, changing the salt concentration, the application of organic solvents, and the application of molecules that compete with the bound ligand. Preferably, the means employed for elution will release the compound from the target gene product of invention, but will not effect the interaction between the affinity tag and the solid support, thereby achieving selective elution of test compound.

In a preferred embodiment, affinity chromatography is used for high through put screening. In this embodiment, the test compound is detectably labeled (e.g., with fluorescent dyes, radioactive isotopes, etc.) and applied to polypeptide molecules of the invention in a spatially addressed fashion (e.g., attached to separate wells of a microplate). Binding between the test compound and the polypeptide molecule of the invention can be determined by the presence of the detectable label on the test compound to quickly identify which wells contain test compounds capable of binding.

In practice, microtiter plates are conveniently utilized as the solid phase. The anchored component is immobilized by non-covalent or covalent attachments. Non-covalent attachment can be accomplished by simply coating the solid surface with a solution of the protein and drying the coated surface. Alternatively, an immobilized antibody, preferably a monoclonal antibody, specific for the protein to be immobilized is used to anchor the protein to the solid surface. The surfaces are prepared in advance and stored. In order to conduct the assay, the nonimmobilized component is added to the coated surface containing the anchored component. After the reaction is complete, unreacted components are removed (e. g., by washing) under conditions such that any complexes formed will remain immobilized on the solid surface. The detection of complexes anchored on the solid surface is accomplished in a number of ways. Where the previously nonimmobilized component is pre-labeled, the detection of label immobilized on the surface indicates that complexes were formed. Where the previously nonimmobilized component is not pre-labeled, an indirect label is used to detect complexes anchored on the surface; e.g., using a labeled antibody specific for the previously nonimmobilized component (the antibody, in turn, is directly labeled or indirectly labeled with a labeled anti-Ig antibody).

Alternatively, a reaction is conducted in a liquid phase, the reaction products are separated from unreacted components, and complexes are detected; e.g., using an immobilized antibody specific for the target gene product or for the test compound, to anchor complexes formed in solution, and a second labeled antibody, specific for the other component of the complex to allow detection of anchored complexes.

In yet another embodiment, a scintillation proximity assay (“SPA”) can be used to identify test compounds capable of binding to a target gene product of the invention. In this embodiment either the target gene product of the invention or the test compound must labeled (e.g., with a radioisotope, etc.). The unlabeled entity is attached to a surface impregnated with a scintillant. The labeled entity is then incubated with the attached unlabeled entity under conditions that allow binding. The amount of binding between a target gene product of the invention and test compound is quantitated with a scintillation counter (Cook, 1996, Drug Discov. Today 1:287-294; Mei et al., 1997, Bioorg. Med. Chem. 5:1173-1184; Mei et al., 1998, Biochemistry 37:14204-14212). High throughput SPA screening uses microplates with scintillant either directly incorporated into the plastic (Nakayama et al., 1998, J. Biomol. Screening 3:43-48) or coating the plastic. In a preferred embodiment, such microtiter plates are used in methods of the invention comprising (a) labeling of the target gene product of the invention with a radioactive label; (b) contacting the labeled target gene product with a test compound, wherein the test compound is attached to a microtiter well coated with scintillant; and (c) identifying and quantifying the amount of polypeptide of the invention bound to the test compound with SPA.

In yet another embodiment, nuclear magnetic resonance spectroscopy (“NMR”) is used to identify test compounds capable of binding target gene product of the invention. NMR is used to identify target gene product of the invention that are bound by a test compound by qualitatively determining changes in chemical shift, specifically from distances measured using relaxation effects. NMR-based approaches have been used in the identification of small molecule binders of protein drug targets (Xavier et al., 2000, Trends Biotechnol. 18:349-356). Also applicable is a strategy for lead generation by NMR using a library of small molecules which has been described in Fejzo et al. (1999, Chem. Biol. 6:755-769).

In yet another embodiment, fluorescence resonance energy transfer (“FRET”) can be used to identify test compounds capable of binding to a target gene product of the invention. In this embodiment, both the target gene product of the invention and the test compound are labeled with a different fluorescent molecule (i.e., flourophore). A characteristic change in fluorescence occurs when two fluorophores with overlapping emission and excitation wavelength bands are held together in close proximity, such as by a binding event. One of the fluorophores used as a label will have overlapping excitation and emission spectra with the other fluorophore used as a label such that one fluorophore (the donor) transfers its emission energy to excite the other fluorophore (the acceptor). The acceptor preferably emits light of a different wavelength upon relaxing to the ground state, or relaxes non-radioactively to quench fluorescence. FRET is very sensitive to the distance between the two fluorophores, and allows measurement of molecular distances less than 10 nm (e.g., U.S. Pat. No. 6,337,183 and Matsumoto et al., 2000, Bioorg. Med. Chem. Lett. 10:1857-1861).

5.4.1.1 Assays For Compounds That Affect Target Gene Product Interactions

The target gene products of the invention interact, in vivo, with one or more cellular or extracellular macromolecules, such as proteins. Such macromolecules include, but are not limited to, nucleic acid molecules and proteins identified via methods such as those described above. For purposes of this discussion, such cellular and extracellular macromolecules are referred to herein as “binding partners.” Compounds that disrupt such interactions can be useful in regulating the activity of the target gene protein, especially mutant target gene proteins. Such compounds include, but are not limited to molecules such as antibodies, peptides, and the like, as described.

Any method suitable for detecting or measuring protein-macromolecule interactions can be employed for identifying novel target gene product-cellular or extracellular protein interactions as well as compounds that interfere with such interactions. Many of the techniques described in the previous section can be used for this purpose.

The basic principle of the assay systems used to identify compounds that interfere with the interaction between the target gene product and its cellular or extracellular binding partner or partners involves preparing a reaction mixture containing the target gene product and the binding partner under conditions and for a time sufficient to allow the two to interact and bind, thus forming a complex. In order to test a compound for inhibitory activity, the reaction mixture is prepared in the presence and absence of the test compound. The test compound is initially included in the reaction mixture, or added at a time subsequent to the addition of target gene product and its cellular or extracellular binding partner. Control reaction mixtures are incubated without the test compound. The formation of complexes between the target gene protein and the cellular or extracellular binding partner is then detected. The formation of a complex in the control reaction, but not in the reaction mixture containing the test compound, indicates that the compound interferes with the interaction of the target gene protein and the interactive binding partner. Additionally, complex formation within reaction mixtures containing the test compound and normal target gene protein can also be compared to complex formation within reaction mixtures containing the test compound and a mutant target gene protein. This comparison can be important in those cases wherein it is desirable to identify compounds that disrupt intermolecular interactions involving mutant but not normal target gene proteins.

The assay for compounds that interfere with the interaction of the target gene products and binding partners is conducted in either a heterogeneous or a homogeneous format. Heterogeneous assays involve anchoring either the target gene product or the binding partner onto a solid phase and detecting complexes anchored on the solid phase at the end of the reaction. In homogeneous assays, the entire reaction is carried out in a liquid phase. In either approach, the order of addition of reactants is varied to obtain different information about the compounds being tested. For example, test compounds that interfere with the interaction between the target gene products and the binding partners, e.g., by competition, are identified by conducting the reaction in the presence of the test substance; i.e., by adding the test substance to the reaction mixture prior to or simultaneously with the target gene protein and an interacting cellular or extracellular binding partner. Alternatively, test compounds that disrupt preformed complexes, e.g. compounds with higher binding constants that displace one of the components from the complex, are tested by adding the test compound to the reaction mixture after complexes have been formed. The various formats are described briefly below.

In a heterogeneous assay system, either the target gene protein or the interactive cellular or extracellular binding partner, is anchored onto a solid surface, while the non-anchored species is labeled, either directly or indirectly. In practice, microtiter plates are conveniently utilized. The anchored species is immobilized either by non-covalent or covalent attachment. Non-covalent attachment is accomplished simply by coating the solid surface with a solution of the target gene product or binding partner and drying the coated surface. Alternatively, an immobilized antibody specific for the species to be anchored is used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface is accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, is directly labeled or indirectly labeled with a labeled anti-Ig antibody). Depending upon the order of addition of reaction components, test compounds which inhibit complex formation or which disrupt preformed complexes are detected.

Alternatively, the reaction is conducted in a liquid phase in the presence or absence of the test compound, the reaction products separated from unreacted components, and complexes detected; e.g., using an immobilized antibody specific for one of the binding components to anchor any complexes formed in solution, and a second, labeled antibody specific for the other partner to detect anchored complexes. Again, depending upon the order of addition of reactants to the liquid phase, test compounds which inhibit complex or which disrupt preformed complexes are identified.

In an alternate embodiment of the invention, a homogeneous assay can be used. In this approach, a preformed complex of the target gene protein and the interacting cellular or extracellular binding partner is prepared in which either the target gene product or its binding partner is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex results in the generation of a signal above background. In this way, test substances which disrupt target gene protein/cellular or extracellular binding partner interaction are identified.

In a particular embodiment, the target gene product is prepared for immobilization using recombinant DNA techniques described above. For example, the target gene coding region is fused to a glutathione-S-transferase (GST) gene using a fusion vector, such as pGEX-5X-1, in such a manner that its binding activity is maintained in the resulting fusion protein. The interactive cellular or extracellular binding partner is purified and used to raise a monoclonal antibody, using methods routinely practiced in the art and as described above. This antibody is labeled with the radioactive isotope ¹²⁵I, for example, by methods routinely practiced in the art. In a heterogeneous assay, e.g., the GST-target gene fusion protein is anchored to glutathione-agarose beads. The interactive cellular or extracellular binding partner is then added in the presence or absence of the test compound in a manner that allows interaction and binding to occur. At the end of the reaction period, unbound material can be washed away, and the labeled monoclonal antibody is added to the system and allowed to bind to the complexed components. The interaction between the target gene protein and the interactive cellular or extracellular binding partner is detected by measuring the amount of radioactivity that remains associated with the glutathione-agarose beads. A successful inhibition of the interaction by the test compound results in a decrease in measured radioactivity.

Alternatively, the GST-target gene fusion protein and the interactive cellular or extracellular binding partner are mixed together in liquid in the absence of the solid glutathione-agarose beads. The test compound is added either during or after the species are allowed to interact. This mixture is added to the glutathione-agarose beads and unbound material is washed away. Again the extent of inhibition of the target gene product/binding partner interaction is detected by adding the labeled antibody and measuring the radioactivity associated with the beads.

In another embodiment of the invention, these same techniques are employed using peptide fragments that correspond to the binding domains of the target gene product and/or the interactive cellular or extracellular binding partner (in cases where the binding partner is a protein), in place of one or both of the full length proteins. Any number of methods routinely practiced in the art are used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex are then selected. Sequence analysis of the genes encoding the respective proteins reveals the mutations that correspond to the region of the protein involved in interactive binding. Alternatively, one protein is anchored to a solid surface using methods described above, and allowed to interact with and bind to its labeled binding partner, which has been treated with a proteolytic enzyme, such as trypsin. After washing, a short, labeled peptide comprising the binding domain remains associated with the solid material, and can be isolated and identified by amino acid sequencing. Also, once the gene coding for the cellular or extracellular binding partner is obtained, short gene segments are engineered to express peptide fragments of the protein, which are tested for binding activity and purified or synthesized.

For example, and not by way of limitation, a target gene product is anchored to a solid material as described, above, by making a GST-target gene fusion protein and allowing it to bind to glutathione agarose beads. The interactive cellular or extracellular binding partner is labeled with a radioactive isotope, such as ³⁵S, and cleaved with a proteolytic enzyme such as trypsin. Cleavage products are added to the anchored GST-target gene fusion protein and allowed to bind. After washing away unbound peptides, labeled bound material, representing the cellular or extracellular binding partner binding domain, is eluted, purified, and analyzed for amino acid sequence by well known methods. Peptides so identified are produced synthetically or fused to appropriate facilitative proteins using well known recombinant DNA technology.

5.4.1.2 Screening a Combinatorial Chemical library

In one embodiment of the present invention, the proteins encoded by the fungal genes identified using the methods of the present invention are isolated and expressed. These recombinant proteins are then used as targets in assays to screen libraries of compounds for potential drug candidates. The generation of chemical libraries is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds theoretically can be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop et al., “Applications of Combinatorial Technologies to Drug Discovery, Background and Peptide Combinatorial Libraries,” Journal of Medicinal Chemistry, Vol. 37, No. 9, 1233-1250 (1994). Other chemical libraries known to those in the art may also be used, including natural product libraries.

Once generated, combinatorial libraries are screened for compounds that possess desirable biological properties. For example, compounds which may be useful as drugs or to develop drugs would likely have the ability to bind to the target protein identified, expressed and purified as discussed above. Further, if the identified target protein is an enzyme, candidate compounds would likely interfere with the enzymatic properties of the target protein. For example, the enzymatic function of a target protein may be to serve as a protease, nuclease, phosphatase, dehydrogenase, transporter protein, transcriptional enzyme, replication component, and any other type of enzyme known or unknown. Thus, the present invention contemplates using the protein products described above to screen combinatorial chemical libraries.

In some embodiments of the present invention, the biochemical activity of the protein, as well as the chemical structure of a substrate on which the protein acts is known. In other embodiments of the present invention, the biochemical activity of the target protein is unknown and the target protein has no known substrates.

In some embodiments of the present invention, libraries of compounds are screened to identify compounds that function as inhibitors of the target gene product. First, a library of small molecules is generated using methods of combinatorial library formation well known in the art. U.S. Pat. Nos. 5,463,564 and 5,574,656, to Agrafiotis, et al., entitled “System and Method of Automatically Generating Chemical Compounds with Desired Properties,” the disclosures of which are incorporated herein by reference in their entireties, are two such teachings. Then the library compounds are screened to identify those compounds that possess desired structural and functional properties. U.S. Pat. No. 5,684,711, the disclosure of which is incorporated herein by reference in its entirety, also discusses a method for screening libraries.

To illustrate the screening process, the target gene product, an enzyme, and chemical compounds of the library are combined and permitted to interact with one another. A labeled substrate is added to the incubation. The label on the substrate is such that a detectable signal is emitted from metabolized substrate molecules. The emission of this signal permits one to measure the effect of the combinatorial library compounds on the enzymatic activity of target enzymes by comparing it to the signal emitted in the absence of combinatorial library compounds. The characteristics of each library compound are encoded so that compounds demonstrating activity against the enzyme can be analyzed and features common to the various compounds identified can be isolated and combined into future iterations of libraries.

Once a library of compounds is screened, subsequent libraries are generated using those chemical building blocks that possess the features shown in the first round of screen to have activity against the target enzyme. Using this method, subsequent iterations of candidate compounds will possess more and more of those structural and functional features required to inhibit the function of the target enzyme, until a group of enzyme inhibitors with high specificity for the enzyme can be found. These compounds can then be further tested for their safety and efficacy as antibiotics for use in mammals.

It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in PCT publications No. WO9935494, WO9819162, WO9954728, the disclosures of which are incorporated herein by reference in their entireties.

Similar methods may be used to identify compounds which inhibit the activity of proteins from organisms other than C. neoformans which are homologous to the C. neoformans target proteins described herein. For example, the proteins may be from animal fugal pathogens such as Aspergillus niger, Aspergillus flavis, Candida tropicalis, Candida parapsilopsis, Candida krusei, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the proteins are from an organism other than Saccharomyces cerevisiae Candida albicans, or Aspergillus fumigatus.

5.4.1.3 In vitro Enzyme Assays

C. neoformans strains are used to develop in vitro assays for biochemical activities shown to be essential to cell viability, e.g., by homology to known essential genes of Candida albicans and/or Aspergillus fumigatus. A number of such essential genes identified by sequence analysis of the C. neoformans genome display statistically significant similarity to biochemically characterized gene products from other organisms.

Therefore, a number of well characterized standard in vitro biochemical assays (e.g., DNA binding, RNA processing, GTP binding and hydrolysis, and phosphorylation) are readily adapted for these validated drug targets. Alternatively, novel assays are developed using biochemical information pertaining to validated drug targets within the C. neoformans sequenced gene collection. Any assays known in the art for enzymes with similar biochemical activities (e.g., mechanism of action, class of substrate) are adapted for screening for inhibitors of the enzymes encoded by these essential C. neoformans genes.

The present invention also provides cell extracts useful in establishing in vitro assays for suitable biochemical targets. For example, in an embodiment of the present invention, conditional-expression C. neoformans mutant strains are grown either under constitutive expression conditions or transcription repression conditions to either overproduce or deplete a particular gene product. Cellular extracts resulting from strains incubated under these two conditions are compared with extracts prepared from identically-grown wild type strains. These extracts are then used for the rapid evaluation of targets using existing in vitro assays or new assays directed toward novel gene products, without having to purify the gene product. Such a whole cell extract approach to in vitro assay development is typically necessary for targets involved in cell wall biosynthetic pathways (e.g., (1,3)-β-glucan synthesis or chitin synthesis) which involve multiple gene products that transit the secretory pathway before receiving essential post-translational modifications required for their functional activity. Conditional-expression C. neoformans mutant strains for conditional expression of target genes involved in these, or other cell wall pathways (e.g., (1,6)-β-glucan synthesis) enable in vitro assays to be performed directly in C. neoformans.

5.4.2 Cell-based Screening Assays

Current cell-based assays used to identify or to characterize compounds for drug discovery and development frequently depend on detecting the ability of a test compound to modulate the activity of a target molecule located within a cell or located on the surface of a cell. Most often such target molecules are proteins such as enzymes, receptors and the like. However, target molecules also include other molecules such as DNAs, lipids, carbohydrates and RNAs including messenger RNAs, ribosomal RNAs, tRNAs and the like. A number of highly sensitive cell-based assay methods are available to those of skill in the art to detect binding and interaction of test compounds with specific target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment such as the periplasm of a bacterial cell. Thus, current cell-based assay methods are limited in that they are not effective in identifying or characterizing compounds that interact with their targets with moderate to low affinity or compounds that interact with targets that are not readily accessible.

The cell-based assay methods of the present invention have substantial advantages over current cell-based assays. These advantages derive from the use of sensitized cells in which the level or activity of at least one gene product required for fungal survival, growth, proliferation, virulence, or pathogenicity (the target molecule) has been specifically reduced to the point where the presence or absence of its function becomes a rate-determining step for fungal survival, growth, proliferation, virulence, or pathogenicity. Such sensitized cells become much more sensitive to compounds that are active against the affected target molecule. For example, sensitized cells are obtained by growing a conditional-expression C. neoformans mutant strain in the presence of a concentration of inducer or repressor which provides a level of a gene product required for fungal growth, survival, proliferation, virulence, or pathogenicity such that the presence or absence of its function becomes a rate-determining step for fungal growth, survival, proliferation, virulence, or pathogenicity. Thus, cell-based assays of the present invention are capable of detecting compounds exhibiting low or moderate potency against the target molecule of interest because such compounds are substantially more potent on sensitized cells than on non-sensitized cells. The effect may be such that a test compound may be two to several times more potent, at least 10 times more potent, at least 20 times more potent, at least 50 times more potent, at least 100 times more potent, at least 1000 times more potent, or even more than 1000 times more potent when tested on the sensitized cells as compared to the non-sensitized cells.

Due in part to the increased appearance of antibiotic resistance in pathogenic microorganisms and to the significant side-effects associated with some currently used antibiotics, novel antibiotics acting at new targets are highly sought after in the art. Yet, another limitation in the current art related to cell-based assays is the problem of repeatedly identifying hits against the same kinds of target molecules in the same limited set of biological pathways. This may occur when compounds acting at such new targets are discarded, ignored or fail to be detected because compounds acting at the Aold@ targets are encountered more frequently and are more potent than compounds acting at the new targets. As a result, the majority of antibiotics in use currently interact with a relatively small number of target molecules within an even more limited set of biological pathways.

The use of sensitized cells of the current invention provides a solution to the above problems in two ways. First, desired compounds acting at a target of interest, whether a new target or a previously known but poorly exploited target, can now be detected above the “noise” of compounds acting at the “old” targets due to the specific and substantial increase in potency of such desired compounds when tested on the sensitized cells of the current invention. Second, the methods used to sensitize cells to compounds acting at a target of interest may also sensitize these cells to compounds acting at other target molecules within the same biological pathway. For example, expression of a gene encoding a ribosomal protein at a level such that the function of the ribosomal protein becomes rate limiting for fungal growth, survival, proliferation, virulence, or pathogenicity is expected to sensitize the cell to compounds acting at that ribosomal protein to compounds acting at any of the ribosomal components (proteins or rRNA) or even to compounds acting at any target which is part of the protein synthesis pathway. Thus an important advantage of the present invention is the ability to reveal new targets and pathways that were previously not readily accessible to drug discovery methods.

Sensitized cells of the present invention are prepared by reducing the activity or level of a target molecule. The target molecule may be a gene product, such as an RNA or polypeptide produced from the nucleic acids required for fungal growth, survival, proliferation, virulence, or pathogenicity described herein. In addition, the target may be an RNA or polypeptide in the same biological pathway as the nucleic acids required for fungal growth, survival, proliferation, virulence, or pathogenicity as described herein. Such biological pathways include, but are not limited to, enzymatic, biochemical and metabolic pathways as well as pathways involved in the production of cellular structures such as the cell membrane.

Current methods employed in the arts of medicinal and combinatorial chemistries are able to make use of structure-activity relationship information derived from testing compounds in various biological assays including direct binding assays and cell-based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus; more compounds can now be characterized providing more useful information than would be obtained otherwise.

Thus, it is now possible using cell-based assays of the present invention to identify or characterize compounds that previously would not have been readily identified or characterized including compounds that act at targets that previously were not readily exploited using cell-based assays. The process of evolving potent drug leads from initial hit compounds is also substantially improved by the cell-based assays of the present invention because, for the same number of test compounds, more structure-function relationship information is likely to be revealed.

The method of sensitizing a cell entails selecting a suitable gene. A suitable gene is one whose expression is required for the growth, survival, proliferation, virulence, or pathogenicity of the cell to be sensitized. The next step is to obtain a cell in which the level or activity of the target can be reduced to a level where it is rate limiting for growth, survival, proliferation, virulence or pathogenicity. For example, the cell may be a conditional-expression C. neoformans mutant strain in which the selected gene is under the control of a regulatable promoter. The amount of RNA transcribed from the selected gene is limited by varying the concentration of an inducer or repressor which acts on the regulatable promoter, thereby varying the activity of the promoter driving transcription of the RNA. Thus, cells are sensitized by exposing them to an inducer or repressor concentration that results in an RNA level such that the function of the selected gene product becomes rate limiting for fungal growth, survival, proliferation, virulence, or pathogenicity.

In one embodiment of the cell-based assays, conditional-expression C. neoformans mutant strains, in which the sequences required for fungal survival, growth, proliferation, virulence, or pathogenicity of C. neoformans described herein are under the control of a regulatable promoter, are grown in the presence of a concentration of inducer or repressor which causes the function of the gene products encoded by these sequences to be rate limiting for fungal growth, survival, proliferation, virulence, or pathogenicity. To achieve that goal, a growth inhibition dose curve of inducer or repressor is calculated by plotting various doses of inducer or repressor against the corresponding growth inhibition caused by the limited levels of the gene product required for fungal proliferation. From this dose-response curve, conditions providing various growth rates, from 1 to 100% as compared to inducer or repressor-free growth, can be determined. For example, if the regulatable promoter is repressed by tetracycline, the conditional-expression C. neoformans mutant strain may be grown in the presence of varying levels of tetracyline. Similarly, inducible promoters may be used. In this case, the conditional-expression C. neoformans mutant strains are grown in the presence of varying concentrations of inducer. For example, the highest concentration of the inducer or repressor that does not reduce the growth rate significantly can be estimated from the dose-response curve. Cellular proliferation can be monitored by growth medium turbidity via OD measurements. In another example, the concentration of inducer or repressor that reduces growth by 25% can be predicted from the dose-response curve. In still another example, a concentration of inducer or repressor that reduces growth by 50% can be calculated from the dose-response curve. Additional parameters such as colony forming units (cfu) are also used to measure cellular growth, survival and/or viability.

In specific embodiments of the present invention, a diploid mutant strain of C. neoformans may similarly be used as the basis for detection of an antifungal or therapeutic agent.

In various embodiments, the conditional-expression C. neoformans mutant strain is grown under a first set of conditions where the heterologous promoter is expressed at a relatively low level (i.e. partially repressed) and the extent of growth determined. This experiment is repeated in the presence of a test compound and a second measurement of growth obtained. The extent of growth in the presence and in the absence of the test compound are then compared to provide a first indicator value. Two further experiments are performed, using non-repressing growth conditions where the target gene is expressed at substantially higher levels than in the first set of conditions. The extent of growth is determined in the presence and absence of the test compound under the second set of conditions to obtain a second indicator value. The first and second indicator values are then compared. If the indicator values are essentially the same, the data suggest that the test compound does not inhibit the test target. However, if the two indicator values are substantially different, the data indicates that the level of expression of the target gene product may determine the degree of inhibition by the test compound and, therefore, it is likely that the gene product is the target of that test compound. Whole-cell assays comprising collections or subsets of multiple sensitized strains may also be screened, for example, in a series of 96-well, 384-well, or even 1586-well microtiter plates, with each well containing individual strains sensitized to identify compounds displaying a preferential activity against each affected target comprising a target set or subset selected from, but not limited to the group consisting of fungal-specific, pathogen-specific, desired biochemical-function, human-homolog, cellular localization, and signal transduction cascade target sets.

Cells to be assayed are exposed to the above-determined concentrations of inducer or repressor. The presence of the inducer or repressor at this sub-lethal concentration reduces the amount of the proliferation-required gene product to the lowest amount in the cell that will support growth. Cells grown in the presence of this concentration of inducer or repressor are therefore specifically more sensitive to inhibitors of the proliferation-required protein or RNA of interest as well as to inhibitors of proteins or RNAs in the same biological pathway as the proliferation-required protein or RNA of interest but not specifically more sensitive to inhibitors of unrelated proteins or RNAs.

Cells pretreated with sub-inhibitory concentrations of inducer or repressor, which therefore contain a reduced amount of proliferation-required target gene product, are used to screen for compounds that reduce cell growth. The sub-lethal concentration of inducer or repressor may be any concentration consistent with the intended use of the assay to identify candidate compounds to which the cells are more sensitive than are control cells in which this gene product is not rate-limiting. For example, the sub-lethal concentration of the inducer or repressor may be such that growth inhibition is at least about 5%, at least about 8%, at least about 10%, at least about 20%, at least about 30%, at least about 40%, at least about 50%, at least about 60% at least about 75%, at least 80%, at least 90%, at least 95% or more than 95%. Cells which are pre-sensitized using the preceding method are more sensitive to inhibitors of the target protein because these cells contain less target protein to inhibit than wild-type cells.

It will be appreciated that similar methods may be used to identify compounds which inhibit virulence or pathogenicity. In such methods, the virulence or pathogenicity of cells exposed to the candidate compound which express rate limiting levels of a gene product involved in virulence or pathogenicity is compared to the virulence or pathogenicity of cells exposed to the candidate compound in which the levels of the gene product are not rate limiting. Virulence or pathogenicity may be measured using the techniques described herein.

In another embodiment of the cell-based assays of the present invention, the level or activity of a gene product required for fungal growth, survival, proliferation, virulence, or pathogenicity is reduced using a mutation, such as a temperature sensitive mutation, in the sequence required for fungal growth, survival, proliferation, virulence, or pathogenicity and an inducer or repressor level which, in conjunction with the temperature sensitive mutation, provides levels of the gene product required for fungal growth, survival, proliferation, virulence, or pathogenicity which are rate limiting for proliferation. Growing the cells at an intermediate temperature between the permissive and restrictive temperatures of the temperature sensitive mutant where the mutation is in a gene required for fungal growth, survival, proliferation, virulence, or pathogenicity produces cells with reduced activity of the gene product required for growth, survival, proliferation, virulence, or pathogenicity. The concentration of inducer or repressor is chosen so as to further reduces the activity of the gene product required for fungal growth, survival, proliferation, virulence, or pathogenicity. Drugs that may not have been found using either the temperature sensitive mutation or the inducer or repressor alone may be identified by determining whether cells in which expression of the nucleic acid encoding the proliferation-required gene product has been reduced and which are grown at a temperature between the permissive temperature and the restrictive temperature are substantially more sensitive to a test compound than cells in which expression of the gene product required for fungal growth, survival, proliferation, virulence, or pathogenicity has not been reduced and which are grown at a permissive temperature. Also drugs found previously from either the use of the inducer or repressor alone or the temperature sensitive mutation alone may have a different sensitivity profile when used in cells combining the two approaches, and that sensitivity profile may indicate a more specific action of the drug in inhibiting one or more activities of the gene product.

Temperature sensitive mutations may be located at different sites within a gene and may lie within different domains of the protein. For example, the dnaB gene of Escherichia coli encodes the replication fork DNA helicase. DnaB has several domains, including domains for oligomerization, ATP hydrolysis, DNA binding, interaction with primase, interaction with DnaC, and interaction with DnaA. Temperature sensitive mutations in different domains of DnaB confer different phenotypes at the restrictive temperature, which include either an abrupt stop or a slow stop in DNA replication either with or without DNA breakdown (Wechsler, J. A. and Gross, J. D. 1971 Escherichia coli mutants temperature-sensitive for DNA synthesis. Mol. Gen. Genetics 113:273-284) and termination of growth or cell death. Thus, temperature sensitive mutations in different domains of the protein may be used in conjunction with conditional-expression C. neoformans mutant strains in which expression of the protein is under the control of a regulatable promoter.

It will be appreciated that the above method may be performed with any mutation which reduces but does not eliminate the activity or level of the gene product which is required for fungal growth, survival, proliferation, virulence, or pathogenicity.

When screening for antimicrobial agents against a gene product required for fungal growth, survival, proliferation, virulence, or pathogenicity, growth inhibition, virulence or pathogenicity of cells containing a limiting amount of that gene product can be assayed. Growth inhibition can be measured by directly comparing the amount of growth, measured by the optical density of the culture relative to uninoculated growth medium, between an experimental sample and a control sample. Alternative methods for assaying cell proliferation include measuring green fluorescent protein (GFP) reporter construct emissions, various enzymatic activity assays, and other methods well known in the art. Virulence and pathogenicity may be measured using the techniques described herein.

It will be appreciated that the above method may be performed in solid phase, liquid phase, a combination of the two preceding media, or in vivo. For example, cells grown on nutrient agar containing the inducer or repressor which acts on the regulatable promoter used to express the proliferation required gene product may be exposed to compounds spotted onto the agar surface. A compound's effect may be judged from the diameter of the resulting killing zone, the area around the compound application point in which cells do not grow. Multiple compounds may be transferred to agar plates and simultaneously tested using automated and semi-automated equipment including but not restricted to multi-channel pipettes (for example the Beckman Multimek) and multi-channel spotters (for example the Genomic Solutions Flexys). In this way multiple plates and thousands to millions of compounds may be tested per day.

The compounds are also tested entirely in liquid phase using microtiter plates as described below. Liquid phase screening may be performed in microtiter plates containing 96, 384, 1536 or more wells per microtiter plate to screen multiple plates and thousands to millions of compounds per day. Automated and semi-automated equipment are used for addition of reagents (for example cells and compounds) and for determination of cell density.

The compounds are also tested in vivo using the methods described herein.

It will be appreciated that each of the above cell-based assays may be used to identify compounds which inhibit the activity of gene products from organisms other than C. neoformans which are homologous to the C. neoformans gene products described herein. For example, the target gene products may be from animal fugal pathogens such as Aspergillus niger, Aspergillus flavis, Candida tropicalis, Candida albicans, Candida parapsilopsis, Candida krusei, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae, Candida albicans, and/or Asperigillus fumigatus.

5.4.2.1 Cell-Based Assays Using Conditional-expression C. neoformans Mutant Strains

Conditional-expression C. neoformans mutant strains in which a gene required for fungal survival, growth, proliferation, virulence, or pathogenicity is placed under the control of a regulatable promoter are constructed using the methods described herein. For the purposes of the present example, the regulatable promoter may be the tetracycline regulated promoter described herein, but it will be appreciated that any regulatable promoter may be used, e.g., GAL7 promoter.

In one embodiment of the present invention, an individual conditional-expression C. neoformans mutant strain is used as the basis for detection of a therapeutic agent active against a haploid or diploid pathogenic fungal cell. In this embodiment, the test organism is a conditional-expression C. neoformans mutant strain having a gene that has been modified, by recombination, to place the gene under the controlled expression of a heterologous promoter. This test conditional-expression C. neoformans mutant strain is then grown under a first set of conditions where the heterologous promoter is expressed at a relatively low level (“repressing”) and the extent of growth determined. This measurement may be carried out using any appropriate standard known to those skilled in the art including optical density, wet weight of pelleted cells, total cell count, viable count, DNA content, and the like. This experiment is repeated in the presence of a test compound and a second measurement of growth obtained. The extent of growth in the presence and in the absence of the test compound, which can conveniently be expressed in terms of indicator values, are then compared. A dissimilarity in the extent of growth or indicator values provides an indication that the test compound may interact with the target essential gene product.

To gain more information, two further experiments are performed, using a second set of “non-repressing” growth conditions where the essential gene, under the control of the heterologous promoter, is expressed at a level substantially higher than in the first set of conditions described above. The extent of growth or indicator values is determined in the presence and absence of the test compound under this second set of conditions. The extent of growth or indicator values in the presence and in the absence of the test compound are then compared. A dissimilarity in the extent of growth or indicator values provides an indication that the test compound may interact with the target essential gene product.

Furthermore, the extent of growth in the first and in the second set of growth conditions can also be compared. If the extent of growth is essentially the same, the data suggest that the test compound does not inhibit the gene product encoded by the modified gene carried by the conditional-expression C. neoformans mutant strain tested. However, if the extent of growth are substantially different, the data indicate that the level of expression of the subject gene product may determine the degree of inhibition by the test compound and, therefore, it is likely that the subject gene product is the target of that test compound.

Although each conditional-expression C. neoformans mutant strain can be tested individually, it will be more efficient to screen entire sets or subsets of a conditional-expression C. neoformans mutant strain collection at one time. Therefore in one aspect of this invention, arrays may be established, for example in a series of 96-well microtiter plates, with each well containing a single conditional-expression C. neoformans mutant strain. In one representative, but not limiting approach, four microtiter plates are used, comprising two pairs where the growth medium in one pair supports greater expression of the heterologous promoter controlling the remaining active allele in each strain, than the medium in the other pair of plates. One member of each pair is supplemented with a compound to be tested and measurements of growth of each conditional-expression C. neoformans mutant strain is determined using standard procedures to provide indicator values for each isolate tested. The collection of conditional-expression C. neoformans mutant strains used in such a method for screening for therapeutic agents may comprise a subset of conditional-expression C. neoformans mutant strains selected from, but not limited to the group consisting of fungal-specific, pathogen-specific, desired biochemical-function, human-homolog, cellular localization, and signal transduction cascade target sets.

The conditional-expression C. neoformans mutant strains are grown in medium comprising a range of tetracycline concentrations to obtain the growth inhibitory dose-response curve for each strain. First, seed cultures of the conditional-expression C. neoformans mutant strains are grown in the appropriate medium. Subsequently, aliquots of the seed cultures are diluted into medium containing varying concentrations of tetracycline. For example, the conditional-expression C. neoformans mutant strains may be grown in duplicate cultures containing two-fold serial dilutions of tetracycline. Additionally, control cells are grown in duplicate without tetracycline. The control cultures are started from equal amounts of cells derived from the same initial seed culture of a conditional-expression C. neoformans mutant strain of interest. The cells are grown for an appropriate period of time and the extent of growth is determined using any appropriate technique. For example, the extent of growth may be determined by measuring the optical density of the cultures. When the control culture reaches mid-log phase the percent growth (relative to the control culture) for each of the tetracycline containing cultures is plotted against the log concentrations of tetracycline to produce a growth inhibitory dose response curve for tetracycline. The concentration of tetracycline that inhibits cell growth to 50% (IC₅₀) as compared to the 0 mM tetracyline control (0% growth inhibition) is then calculated from the curve. Alternative methods of measuring growth are also contemplated. Examples of these methods include measurements of proteins, the expression of which is engineered into the cells being tested and can readily be measured. Examples of such proteins include green fluorescent protein (GFP) and various enzymes.

Cells are pretreated with the selected concentration of tetracycline and then used to test the sensitivity of cell populations to candidate compounds. For example, the cells may be pretreated with a concentration of tetracycline which inhibits growth by at least about 5%, at least about 8%, at least about 10%, at least about 20%, at least about 30%, at least about 40%, at least about 50%, at least about 60% at least about 75%, at least 80%, at least 90%, at least 95% or more than 95%. The cells are then contacted with the candidate compound and growth of the cells in tetracycline containing medium is compared to growth of the control cells in medium which lacks tetracycline to determine whether the candidate compound inhibits growth of the sensitized cells (i.e. the cells grown in the presence of tetracycline). For example, the growth of the cells in tetracycline containing medium may be compared to the growth of the cells in medium lacking tetracycline to determine whether the candidate compound inhibits the growth of the sensitized cells (i.e. the cells grown in the presence of tetracyline) to a greater extent than the candidate compound inhibits the growth of cells grown in the absence of tetracycline. For example, if a significant difference in growth is observed between the sensitized cells (i.e. the cells grown in the presence of tetracycline) and the non-sensitized cells (i.e. the cells grown in the absence of tetracycline), the candidate compound may be used to inhibit the proliferation of the organism or may be further optimized to identify compounds which have an even greater ability to inhibit the growth, survival, or proliferation of the organism.

In another embodiment, the screening of the candidate compounds can be performed with conditional-expression C. neoformans mutant cells which reside inside a host cell in the presence or absence of an inducer/repressor, such as tetracycline. In addition to existing as a free-living saprophyte, C. neoformans is also a facultative intracellular pathogen (Feldmesser et al., 2001, Trends in Microbiol;. 9:273) which survive in macrophages during chronic pulmonay infections and replicates in phagocytes in vitro (Lee et al., 1995, Lab. Invest. 73:871-879). The pattern of gene expression of the intracellular form of C. neoformans is expected to differ from the free-living form, and thus presents additional and potentially unique targets for drug screening. Accordingly, the C. neoformans mutant cells can be used to infect an animal, and after a period of time, animal cells of cell types that are known to be infected by C. neoformans are obtained and exposed to the inducer/repressor and candidate compounds as described above. The rate of mutant cells in yeast form budding inside a cell and the number of yeast cells per phagosome can be used to determine the effect of the candidate compound on the mutant cells. Alternatively, an in vitro assays can be performed with host cells, such as murine macrophage cell lines, primary peritoneal and alveolar macropahages, wherein the mutant yeast cells infect the host cells in tissue culture in the presence or absence of an inducer/repressor. The number of mutant cells inside macrophages can be determined by lysing the macrophages and plating the cell supernatant and lysates in the appropriate plate medium for counting. See, for example, methods described in Fries et al., 2001, J. Clin. Invest. 108:1639-1648; and Goldman et al., 1998, Proc. Natl. Acad. Sci. 95:14967-14972; which are incorporated herein by reference in their entireties. Compounds identified in such assays are expected to be able to reach the fungal cells even though they are protected by secreted polysaccharides inside host cells, and may be particularly effective against chronic, recurrent or latent forms of C. neoformans infection.

Similar to the above methods, the virulence or pathogenicity of cells exposed to a candidate compound which express a rate limiting amount of a gene product required for virulence or pathogenicity may be compared to the virulence or pathogenicity of cells exposed to the candidate compound in which the level of expression of the gene product required for virulence or pathogenicity is not rate limiting. In such methods, test animals are challenged with the conditional-expression C. neoformans mutant strain and fed a diet containing the desired amount of tetracycline and the candidate compound. Thus, the conditional-expression C. neoformans mutant strain infecting the test animals expresses a rate limiting amount of a gene product required for virulence or pathogenicity (i.e. the conditional-expression C. neoformans mutant cells in the test animals are sensitized). Control animals are challenged with the conditional-expression C. neoformans mutant strain and are fed a diet containing the candidate compound but lacking tetracycline. The virulence or pathogenicity of the conditional-expression C. neoformans mutant strain in the test animals is compared to that in the control animals. For example, the virulence or pathogenicity of the conditional-expression C. neoformans mutant strain in the test animals may be compared to that in the control animals to determine whether the candidate compound inhibits the virulence or pathogenicity of the sensitized conditional-expression C. neoformans mutant cells (i.e. the cells in the animals whose diet included tetracyline) to a greater extent than the candidate compound inhibits the growth of the conditional-expression C. neoformans mutant cells in animals whose diet lacked tetracycline. For example, if a significant difference in growth is observed between the sensitized conditional-expression C. neoformans mutant cells (i.e. the cells in animals whose diet included tetracycline) and the non-sensitized cells (i.e. the conditional-expression C. neoformans mutant cells animals whose diet did not include tetracycline), the candidate compound may be used to inhibit the virulence or pathogenicity of the organism or may be further optimized to identify compounds which have an even greater ability to inhibit the virulence or pathogenicity of the organism. Virulence or pathogenicity may be measured using the techniques described therein.

It will be appreciated that the above cell-based assays may be used to identify compounds which inhibit the activity of gene products from organisms other than C. neoformans which are homologous to the C. neoformans gene products described herein. For example, the gene products may be from animal fugal pathogens such as Asperigillus fumigatus, Aspergillus niger, Aspergillus flavis, Candida tropicalis, Candida parapsilopsis, Candida krusei, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisae, Candida albicans, and/or Aspergillus fumigatus.

The cell-based assay described above may also be used to identify the biological pathway in which a nucleic acid required for fungal proliferation, virulence or pathogenicity or the gene product of such a nucleic acid lies. In such methods, cells expressing a rate limiting level of a target nucleic acid required for fungal proliferation, virulence or pathogenicity and control cells in which expression of the target nucleic acid is not rate limiting are contacted with a panel of antibiotics known to act in various pathways. If the antibiotic acts in the pathway in which the target nucleic acid or its gene product lies, cells in which expression of target nucleic acid is rate limiting will be more sensitive to the antibiotic than cells in which expression of the target nucleic acid is not rate limiting.

As a control, the results of the assay may be confirmed by contacting a panel of cells in which the levels of many different genes required for proliferation, virulence or pathogenicity, including the target gene, is rate limiting. If the antibiotic is acting specifically, heightened sensitivity to the antibiotic will be observed only in the cells in which the target gene is rate limiting (or cells in which genes in the same pathway as the target gene is rate limiting) but will not be observed generally in which a gene product required for proliferation, virulence or pathogenicity is rate limiting.

It will be appreciated that the above method for identifying the biological pathway in which a nucleic acid required for proliferation, virulence or pathogenicity lies may be applied to nucleic acids from organisms other than C. neoformans which are homologous to the C. neoformans nucleic acids described herein. For example, the nucleic acids may be from animal fugal pathogens such as Aspergillus fumigatus, Aspergillus niger, Aspergillus flavis, Candida tropicalis, Candida albicans, Candida parapsilopsis, Candida krusei, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the nucleic acids are from an organism other than Saccharomyces cerevisae, Candida albicans and/or Aspergillus fumigatus.

Similarly, the above method may be used to determine the pathway on which a test compound, such as a test antibiotic acts. A panel of cells, each of which expresses a rate limiting amount of a gene product required for fungal survival, growth, proliferation, virulence or pathogenicity where the gene product lies in a known pathway, is contacted with a compound for which it is desired to determine the pathway on which it acts. The sensitivity of the panel of cells to the test compound is determined in cells in which expression of the nucleic acid encoding the gene product required for proliferation, virulence or pathogenicity is at a rate limiting level and in control cells in which expression of the gene product required for proliferation, virulence or pathogenicity is not at a rate limiting level. If the test compound acts on the pathway in which a particular gene product required for proliferation, virulence, or pathogenicity lies, cells in which expression of that particular gene product is at a rate limiting level will be more sensitive to the compound than the cells in which gene products in other pathways are at a rate limiting level. In addition, control cells in which expression of the particular gene required for fungal proliferation, virulence or pathogenicity is not rate limiting will not exhibit heightened sensitivity to the compound. In this way, the pathway on which the test compound acts may be determined.

It will be appreciated that the above method for determining the pathway on which a test compound acts may be applied to organisms other than C. neoformans by using panels of cells in which the activity or level of gene products which are homologous to the C. neoformans gene products described herein is rate limiting. For example, the gene products may be from animal fugal pathogens such as Aspergillus fumigatus, Aspergillus niger, Aspergillus flavis, Candida tropicalis, Candida parapsilopsis, Candida krusei, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae, Candida albicans and/or Aspergillus fumigatus.

One skilled in the art will appreciate that further optimization of the assay conditions, such as the concentration of inducer or repressor used to produce rate limiting levels of a gene product required for fungal proliferation, virulence or pathogenicity and/or the growth conditions used for the assay (for example incubation temperature and medium components) may further increase the selectivity and/or magnitude of the antibiotic sensitization exhibited.

It will be appreciated that the above methods for identifying the pathway in which a gene required for growth, survival, proliferation, virulence or pathogenicity lies or the pathway on which an antibiotic acts may be performed using organisms other than C. neoformans in which gene products homologous to the C. neoformans gene products described herein are rate limiting. For example, the gene products may be from animal fugal pathogens such as Aspergillus fumigatus, Aspergillus niger, Aspergillus flavis, Candida albicans, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisae, Candida albicans, and/or Aspergillus fumigatus.

Furthermore, as discussed above, panels of conditional-expression C. neoformans mutant strains may be used to characterize the point of intervention of any compound affecting an essential biological pathway including antibiotics with no known mechanism of action.

Another embodiment of the present invention is a method for determining the pathway against which a test antibiotic compound is active, in which the activity of proteins or nucleic acids involved in pathways required for fungal growth, survival, proliferation, virulence or pathogenicity is reduced by contacting cells with a sub-lethal concentration of a known antibiotic which acts against the protein or nucleic acid. The method is similar to those described above for determining which pathway a test antibiotic acts against, except that rather than reducing the activity or level of a gene product required for fungal proliferation, virulence or pathogenicity by expressing the gene product at a rate limiting amount in a conditional-expression C. neoformans mutant strain, the activity or level of the gene product is reduced using a sub-lethal level of a known antibiotic which acts against the gene product.

Growth inhibition resulting from the presence of sub-lethal concentration of the known antibiotic may be at least about 5%, at least about 8%, at least about 10%, at least about 20%, at least about 30%, at least about 40%, at least about 50%, at least about 60%, or at least about 75%, at least 80%, at least 90%, at least 95% or more than 95%.

Alternatively, the sub-lethal concentration of the known antibiotic may be determined by measuring the activity of the target proliferation-required gene product rather than by measuring growth inhibition.

Cells are contacted with a combination of each member of a panel of known antibiotics at a sub-lethal level and varying concentrations of the test antibiotic. As a control, the cells are contacted with varying concentrations of the test antibiotic alone. The IC₅₀ of the test antibiotic in the presence and absence of the known antibiotic is determined. If the IC₅₀s in the presence and absence of the known drug are substantially similar, then the test drug and the known drug act on different pathways. If the IC₅₀s are substantially different, then the test drug and the known drug act on the same pathway.

Similar methods may be performed using known antibiotics which act on a gene product homologous to the C. neoformans sequences described herein. The homolgous gene product may be from animal fugal pathogens such as Aspergillus niger, Aspergillus flavis, Candida tropicalis, Candida parapsilopsis, Candida krusei, Coccidioides immitis, Exophalia dermatiditis, Fusarium oxysporum, Histoplasma capsulatum, Pneumocystis carinii, Trichosporon beigeli, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisae, Candida albicans and/or Aspergillus fumigatus.

Another embodiment of the present invention is a method for identifying a candidate compound for use as an antibiotic in which the activity of target proteins or nucleic acids involved in pathways required for fungal proliferation, virulence or pathogenicity is reduced by contacting cells with a sub-lethal concentration of a known antibiotic which acts against the target protein or nucleic acid. The method is similar to those described above for identifying candidate compounds for use as antibiotics except that rather than reducing the activity or level of a gene product required for proliferation, virulence or pathogenicity using conditional-expression C. neoformans mutant strains which express a rate limiting level of the gene product, the activity or level of the gene product is reduced using a sub-lethal level of a known antibiotic which acts against the proliferation-required gene product.

The growth inhibition from the sub-lethal concentration of the known antibiotic may be at least about 5%, at least about 8%, at least about 10%, at least about 20%, at least about 30%, at least about 40%, at least about 50%, at least about 60%, or at least about 75%, or more.

Alternatively, the sub-lethal concentration of the known antibiotic may be determined by measuring the activity of the target proliferation-required gene product rather than by measuring growth inhibition.

In order to characterize test compounds of interest, cells are contacted with a panel of known antibiotics at a sub-lethal level and one or more concentrations of the test compound. As a control, the cells are contacted with the same concentrations of the test compound alone. The IC₅₀ of the test compound in the presence and absence of the known antibiotic is determined. If the IC₅₀ of the test compound is substantially different in the presence and absence of the known drug then the test compound is a good candidate for use as an antibiotic. As discussed above, once a candidate compound is identified using the above methods its structure may be optimized using standard techniques such as combinatorial chemistry.

Similar methods may be performed using known antibiotics which act on a gene product homologous to the C. neoformans sequences described herein. The homolgous gene product may be from animal fugal pathogens such as Aspergillus niger, Aspergillus flavis, Candida albicans, Candida tropicalis, Candida parapsilopsis, Candida krusei, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisae, Candida albicans, and/or Aspergillus fumigatus.

In another embodiment of the present invention, all potential drug targets of a pathogen could be screened simultaneously against a library of compounds using, for example a 96 well microtiter plate format, where growth, measured by optical density or pellet size after centrifugation, may be determined for each well. A genomic approach to drug screening eliminates reliance upon potentially arbitrary and artificial criteria used in evaluating which target to screen and instead allows all potential targets to be screened. This approach not only offers the possibility of identifying specific compounds which inhibit a preferred process (e.g. cell wall biosynthetic gene products) but also the possibility of identifying all fungicidal compounds within that library and linking them to their cognate cellular targets.

In still another embodiment of the present invention, conditional-expression C. neoformans mutant strains could be screened to identify synthetic lethal mutations, and thereby uncover a potentially novel class of drug targets of significant therapeutic value. For example two separate genes may encode homologous proteins that participate in a common and essential cellular function, where the essential nature of this function will only become apparent upon inactivation of both family members. Accordingly, examination of the null phenotype of each gene separately would not reveal the essential nature of the combined gene products, and consequently, this potential drug target would not be identified. Provided the gene products are highly homologous to one another, compounds found to inhibit one family member are likely to inhibit the other and are therefore predicted to approximate the synthetic growth inhibition demonstrated genetically. In other cases however, synthetic lethality may uncover seemingly unrelated (and often nonessential) processes, which when combined produce a synergistic growth impairment (cell death). For example, although disruption of the S. cerevisiae gene RVS161 does not present any discernable vegetative growth phenotype in yeast carrying this single mutation, at least 9 other genes are known to display a synthetic lethal effect when combined with inactivation of RVS161. These genes participate in processes ranging from cytoskeletal assembly and endocytosis, to signal transduction and lipid metabolism and identifies multiple avenues to pursuing a combination drug target strategy. A directed approach to uncovering synthetic lethal interactions with essential and nonessential drug targets is now performed where a conditional-expression C. neoformans mutant strain is identified as displaying an enhanced sensitivity to the tested compound, not because it expresses a reduced level of activity for the drug target, but because its mutation is synthetically lethal in combination with inhibition of a second drug target. Discerning whether the compound specifically inhibits the drug target in the sensitized conditional-expression C. neoformans mutant strain may be achieved by screening the entire conditional-expression C. neoformans mutant strain set for additional mutant strains displaying equal or greater sensitivity to the compound, followed by genetic characterization of a double mutant strain demonstrating synthetic lethality between the two mutations.

5.4.2.2 Screening for Non-antifungal Therapeutic Agents With

Conditional-expression C. neoformans Mutant Strains

The biochemical similarity existing between pathogenic fungi and the mammalian hosts they infect limits the range of clinically useful antimycotic compounds. However, this similarity can be exploited using a conditional-expression C. neoformans mutant strain collection to facilitate the discovery of therapeutics that are not used as antimycotics, but are useful for treatment a wide-range of diseases, such as cancer, inflammation, etc.

In this embodiment of the invention, fungal genes that are homologous to disease-causing genes in an animal or plant, are selected and conditional-expression C. neoformans mutant strains of this set of genes are used for identification of compounds that display potent and specific bioactivity towards the products of these genes, and therefore have potential medicinal value for the treatment of diseases. Essential and non-essential genes and the corresponding conditional-expression C. neoformans mutant strains carrying modified genes are useful in this embodiment of the invention. It has been predicted that many of the genes found within the C. neoformans genome share human functional homologs. It has also been predicted that as many as 1% of human genes are involved in human diseases and therefore may serve as potential drug targets. Accordingly, many genes within the conditional-expression C. neoformans mutant strain collection are homologs to disease-causing human genes and compounds that specifically inactivate individual members of this gene set may in fact have alternative therapeutic value. The invention provides a pluralities of conditional-expression C. neoformans mutant strains in which the modified alleles are fungal genes that share sequence, structural and/or functional similarities to genes that are associated with one or more diseases of the animal or plant.

For example, much of the signal transduction machinery that promotes cell cycle progression and is often perturbed in a variety of cancers is conserved in fungi. Many of these genes encode for cyclins, cyclin-dependent kinases (CDK), CDK inhibitors, phosphatases, and transcription factors that are both structurally and functionally related. As a result, compounds found to display specificity towards any of these functional classes of proteins could be evaluated by secondary screens to test for potential anticancer activity. However, cytotoxic compounds identified in this way need not act on cancer causing targets to display therapeutic potential. For example the taxol family of anti-cancer compounds, which hold promise as therapeutics for breast and ovarian cancers, bind tubulin and promote microtubule assembly, thereby disrupting normal microtubule dynamics. Yeast tubulin displays similar sensitivity to taxol, suggesting that additional compounds affecting other fundamental cellular processes shared between yeast and man could similarly be identified and assessed for antitumor activity.

The phenomenon of pathogenesis extends far beyond the taxonomic borders of microbes and ultimately reflects the underlying physiology. In many ways, the phenomenon of cancer is analogous to the process of pathogenesis by an opportunistic pathogen such as C. neoformans. Both are non-infectious diseases caused by either the body's own cells, or microbes from its natural fauna. These cells grow in a manner unchecked by the immune system and in both cases disease manifests itself by colonization of vital organs and eventual tissue damage resulting in death. Effective drug-based treatment is also elusive for both diseases primarily because the causative agent in both cases is highly related to the host.

In fact, a number of successful therapeutic drugs affecting processes unrelated to cancer have also been discovered through anti-fungal drug screening programs. One clinically-important class of compounds includes the immunosuppressant molecules rapamycin, cyclosporin A, and FK506, which inhibit conserved signal transduction components. Cyclosporin A and FK506, form distinct drug-prolyl isomerase complexes (CyPA- Cyclosporin A and FKBP12-FK506 respectively) which bind and inactivate the regulatory subunit of the calcium and calmodulin-dependent phosphatase, calcineurin. Rapamycin also complexes with FKBP12, but this drug-protein complex also binds to the TOR family of phosphatidylinositol kinases to inhibit translation and cell cycle progression. In each case, both the mechanism of drug action, and the drug targets themselves are highly conserved from yeast to humans.

The identification of C. neoformans drug targets, and grouping the targets into essential-gene, fungal-specific, and pathogen-specific target sets provide the basis for the development of whole-cell screens for compounds that interact with and inhibit individual members of any of these targets. Therefore, similar analyses can be used to identify other sets of conditional-expression C. neoformans mutant strains having modified allelic pairs of genes encoding drug targets with other specific common functions or attributes. For example, conditional-expression C. neoformans mutant strain subsets can be established which comprise gene targets that are highly homologous to human genes, or gene targets that display a common biochemical function, enzymatic activity, or that are involved in carbon compound catabolism, bosynthesis, transport of molecules (transporter activity), cellular localization, signal transduction cascades, cell cycle control, cell adhesion, transcription, translation, DNA replication, etc.

5.4.2.3 Target Gene Dosage-Based Whole Cell Assays

Experiments involving modulating the expression levels of the encoding gene to reveal phenotypes from which gene function may be inferred can be carried out in a pathogenic fungus, such as C. neoformans, using the strains and methods of the present invention. The principle of drug-target-level variation in drug screening involves modulating the expression level of a drug target to identify specific drug resistance or drug sensitivity phenotypes, thereby linking a drug target to a particular compound. Often, these phenotypes are indicative of the target gene encoding the bona fide drug target of this compound. In examples where this is not the case, the candidate target gene may nonetheless provide important insight into the true target gene that is functioning either in a pathway or process related to that inhibited by the compound (e.g. producing synthetic phenotype), or instead functioning as a drug resistance mechanism associated with the identified compound.

The expression level of a given gene product is also elevated by cloning the gene into a plasmid vector that is maintained at multiple copies in the cell. Overexpression of the encoding gene is also achieved by fusing the corresponding open reading frame of the gene product to a more powerful promoter carried on a multicopy plasmid. Using these strategies, a number of overexpression screens have been successfully employed in Saccharomyces cerevisiae to discover novel compounds that interact with characterized drug targets as well as to identify the protein targets bound by existing therapeutic compounds.

In one embodiment, the conditional-expression C. neoformans mutant strain collection of the invention are not only useful in target validation under repressing conditions, but are also useful as a collection of strains overexpressing these same validated drug targets under nonrepressing conditions for whole cell assay development and drug screening.

Variation in the level of expression of a target gene product in a conditional-expression C. neoformans mutant strain is also used to explore resistance to antimycotic compounds. Resistance to existing antifungal therapeutic agents reflects both the limited number of antifungal drugs available and the alarming dependence and reliance clinicians have in prescribing them.

For example, dependence on azole-based compounds such as fluconazole for the treatment of fungal infections, has dramatically undermined the clinical therapeutic value for this compound. The conditional-expression C. neoformans mutant strain collection is used to combat fluconazole resistance by identifying gene products that interact with the cellular target of fluconazole. Such products are used to identify drug targets which, when inactivated in concert with fluconazole, provide a synergistic effect and thereby overcome resistance to fluconazole seen when this compound is used alone. This is accomplished, for example, by using the conditional-expression C. neoformans mutant strain collection to overexpress genes that enhance drug resistance. Such genes include novel or known plasma membrane exporters including ATP-binding cassette (ABC) transporters and multidrug resistance (MDR) efflux pumps, pleiotropic drug resistance (PDR) transcription factors, and protein kinases and phosphatases. Alternatively, genes specifically displaying a differential drug sensitivity are identified by screening conditional-expression C. neoformans mutant strains expressing reduced levels of individual members of the target set. Identifying such genes provides important clues to drug resistance mechanisms that could be targeted for drug-based inactivation to enhance the efficacy of existing antifungal therapeutics.

In another aspect of the present invention, overexpression of the target gene for whole cell assay purposes is supported with promoters other than the tetracycline promoter system. (see Sections 5.3.1)

In another aspect of the present invention, intermediate expression levels of individual drug targets within the conditional-expression C. neoformans mutant strain collection may be engineered to provide strains tailored for the development of unique whole cell assays. In this embodiment of the invention, conditional-expression C. neoformans mutant strains are grown in a medium containing a tetracycline concentration determined to provide only a partial repression of transcription. Under these conditions, it is possible to maintain an expression level between that of the constitutively expressed overproducing strain and that of wild type strain, as well as levels of expression lower than that of the wild-type strain. That is, it is possible to titrate the level of expression to the minimum required for cell viability. By repressing gene expression to this critical state, novel phenotypes, resembling those produced by a partial loss of function mutation (i.e. phenocopies of hypomorphic mutants) may be produced and offer additional target expression levels applicable for whole cell assay development and drug screening. Repressing expression of the remaining allele of an essential gene to the threshold level required for viability, therefore will provide a strain with enhanced sensitivity toward compounds active against this essential gene product.

5.4.2.4 Uses of Tagged strains

In still another aspect of the present invention, one or more unique oligonucleotide sequence tags or “bar codes” are incorporated into individual mutant strains included within a heterozygous strain collection of validated targets. In certain preferred embodiments, two unique sequence tags are incorporated into each conditional-expression C. neoformans mutant strain. The presence of these sequence tags enables an alternative whole cell assay approach to drug screening. Multiple target strains may be screened simultaneously in a mixed population (rather than separately) to identify phenotypes between a particular drug target and its inhibitory agent.

Large-scale parallel analyses are performed using mixed populations of the entire bar coded heterozygous essential strain collection target set and comparing the relative representation of individual strains within a mixed population prior to and after growth in the presence of a compound. Drug-dependent depletion or overrepresentation of a unique bar-coded strain is determined by PCR-amplifying and fluorescently labeling all bar codes within the mixed population and hybridizing the resulting PCR products to an array of complementary oligonucleotides. In preferred embodiments, two sequences tags are incorporated within each conditional-expression C. neoformans mutant strain and, therefore, two signals are generated by hybridization with the array of complementary oligonucleotides. Use of at least two sequence tags therefore provides a more precise determination of the representation of each conditional-expression C. neoformans mutant strain present in the population. Differential representation between bar coded strains indicates gene-specific hypersensitivity or resistance and suggests the corresponding gene product may represent the molecular target of the compound tested.

In one specific embodiment, the mutant strains are conditional-expression C. neoformans mutant strains, and each of the conditional-expression C. neoformans mutant strains of the set comprises at least one, and preferably two unique molecular tags, which, generally, are incorporated within the promoter-replacement cassette used to place the target gene under the control of a heterologous, conditionally-expressed promoter. Each molecular tag is flanked by primer sequences which are common to all members of the set being tested. Growth is carried out in repressive and non-repressive media, in the presence and absence of the compound to be tested. The relative growth of each strain is assessed by carrying out simultaneous PCR amplification of the entire collection of embedded sequence tags.

In one non-limiting aspect of the present invention, the PCR amplification is performed in an asymmetric manner with fluorescent primers and the resulting single stranded nucleic acid product hybridized to an oligonucleotide array fixed to a surface and comprises the entire corresponding set of complementary sequences. Analysis of the level of each fluorescent molecular tag sequence is then determined to estimate the relative amount of growth of the strains in the set, in those media, in the presence and absence of the compound tested.

Therefore, for each conditional-expression C. neoformans mutant strain of the set tested, there could be, in one non-limiting example of this method, four values for the level of the corresponding molecular tag found within the surviving population. They would correspond to cell growth under repressing and non-repressing conditions, both in the presence and absence of the compound being tested. Comparison of growth in the presence and absence of the test compound provides a value or “indicator” for each set of growth media; that is, an indicator derived under repressing and non-repressing conditions. Again, comparison of the two indicator values will reveal if the test compound is active against the gene product expressed by the modified allelic gene pair carried by that specific member of the conditional-expression C. neoformans mutant strain set tested.

In still another aspect of the present invention, each potential drug target gene in this heterozygous tagged or bar-coded collection, may be overexpressed by subsequently introducing either the Tet promoter or another strong, constitutively expressed promoter (e.g. analogs of CaACT1, CaADH1 and CaPGK1 in C. neoformans) upstream of the remaining non-disrupted allele. These constructions allow a further increase in the dosage of the encoded target gene product of individual essential genes to be used in mixed-population drug susceptibility studies. Although overexpression may itself disrupt the normal growth rate of numerous members of the population, reliable comparisons could still be made between mock and drug-treated mixed cultures to identify compound-specific growth differences.

In Saccharomyces cerevisiae, the molecular drug targets of several well-characterized compounds including 3-amino-triazol, benomyl, tunicamycin and fluconazole were identified by a similar approach. In that study, bar-coded strains bearing heterozygous mutations in HIS3, TUB1, ALG7, and ERG11, (i.e. the respective drug targets to the compounds listed above) displayed significantly greater sensitivity when challenged with their respective compound than other heterozygote bar-coded strains when grown together in a mixed population.

In another aspect of the present invention, screens for antifungal compounds can be carried out using complex mixtures of compounds that comprise at least one compound active against the target strain. Tagging or bar-coding the conditional-expression C. neoformans mutant strain collection facilitates a number of large scale analyses necessary to identify gene sets as well as evaluate and ultimately evaluate individual targets within particular gene sets. For example, mixed-population drug screening using a bar-coded conditional-expression C. neoformans mutant strain collection effectively functions as a comprehensive whole cell assay. Minimal amounts of a complex compound library are sufficient to identify compounds that act on individual essential target genes within the collection. This is done without the need to array the collection. Also, strong predictions as to the “richness” of any particular compound library could be made before committing to it in drug screening. It becomes possible then to assess whether, for example, a carbohydrate-based chemical library possesses greater fungicidal activity than a natural product or synthetic compound library. Particularly potent compounds within any complex library of molecules can be immediately identified and evaluated according to the priority of targets and assays available for drug screening. Alternatively, the invention provides applying this information to developing “tailored” screens, in which only those targets which were demonstrated to be inactivated in mixed population experiments by a particular compound library would be included in subsequent array-formatted screens.

Traditionally, drug discovery programs have relied on an individual or a limited set of validated drug targets. The preceding examples emphasize that such an approach is no longer necessary and that high throughput target evaluation and drug screening are now possible. However, a directed approach based on selecting individual targets may still be preferred depending on the expertise, interest, strategy, or budget of a drug discovery program.

5.4.3 Target Evaluation in an Animal or Cell Model System.

Currently, validation of an essential drug target is demonstrated by examining the effect of gene inactivation under standard laboratory conditions. Putative drug target genes deemed nonessential under standard laboratory conditions may be examined within an animal model, for example, by testing the pathogenicity of a strain homozygous for a deletion in the target gene versus wild type. However, essential drug targets are precluded from animal model studies. Therefore, the most desirable drug targets are omitted from the most pertinent conditions to their target evaluation.

In an embodiment of the invention, conditional expression, provided by the conditional-expression C. neoformans mutant essential strain collection, overcomes this longstanding limitation to target validation within a host environment. Animal studies can be performed using mice inoculated with conditional-expression C. neoformans mutant essential strains and examining the effect of gene inactivation by conditional expression. As previously described, the endogenous promoter in the mutant can be replaced with a regulated promoter from C. neoformans or a heterologous promoter. Alternatively, the endogenous promoter can be modified by placing it under the control of a regulatory sequence, such as a tet operator sequence which overlaps with the start site of transcription or the endogenous promoter, and which can be regulated by use of a tet repressor, a tet-VP16 fusion protein, or a mutant tet repressor which display a reverse phenotype when exposed to tetracycline or its analog. Regulation of such promoters will lead to a change in gene product levels sufficient to impair growth of the organism in the animal.

In a preferred embodiment of the invention, the effect on mice injected with a lethal inoculum of a conditional-expression C. neoformans mutant essential strain could be determined depending on whether the mice were provided with an appropriate concentration of tetracycline to inactivate expression of a drug target gene. The lack of expression of a gene demonstrated to be essential under laboratory conditions can thus be correlated with prevention of a terminal C. neoformans infection. In this type of experiment, only mice “treated” with tetracycline-supplemented water, are predicted to survive infection because inactivation of the target gene has killed the conditional-expression C. neoformans mutant strain pathogen within the host. These animal models can be used to investigate whether the infection may be prevented prior to infection (i.e, administering the regulatory molecule such as tetracycline in drinking water prior to infection), or after a full-blown infection has been established (i.e, administer the regulatory molecule such as tetracycline in drinking water after infection is established). The results will indicate whether a target gene is useful for identifying compounds that can be used prophylactically. Furthermore, latency and persistence of infection can be investigated by establishing a latent infection in an animal with the a conditional-expression mutant, administering the regulatory molecule such as atc/tet, and monitoring macrophage occupancy by C. neoformans, or polysaccharide capsule synthesis by C. neoformans.

In yet another embodiment of the invention, conditional expression could be achieved using a temperature-responsive promoter to regulate expression of the target gene or a temperature sensitive allele of a particular drug target, such that the gene is functional at 30° C. but inactivated within the normal body temperature of the mouse.

The conditional-expression C. neoformans mutant strain collection or a desired subset thereof is also well suited for evaluating acquired resistance/suppression or distinguishing between fungicidal/fungistatic phenotypes for an inactivated drug target within an animal model system. In this embodiment of the invention, conditional-expression C. neoformans mutant strains repressed for expression of different essential drug target genes would be inoculated into mice raised on tetracycline-supplemented water. Each of the conditional-expression C. neoformans mutant strains would then be compared according to the frequency of death associated with the different mice populations they infected. It is expected that the majority of infected mice will remain healthy due to fungal cell death caused by tetracycline-dependent inactivation of the essential gene in the conditional-expression C. neoformans mutant strain. However, a conditional-expression C. neoformans mutant strain harboring a drug target more likely to develop extragenic suppressors because it is a fungistatic target rather than fungicidal one, or suppressed by an alternative physiological process active within a host environment, can be identified by the higher incidence of lethal infections detected in mice infected with this particular strain. By this method, it is possible to evaluate/rank the likelihood that individual drug target genes may develop resistance within the host environment.

A number of exemplary animal models of C. neoformans infections that can be used in conjunction with the mutants of the invention are described hereinbelow.

A model in immunocompetent C57B1/6 mice has been established. C57B1/6 mice develop eosinophilic pneumonia in response to pulmonary cryptococcal infection. The model can be used to monitor the severity and progress of the infection via survival, organ fungus burden, serum anticapsular antibody levels, and histopathology by light and electron microscopy (Feldmesser et al., (1998) J Infect Dis 177(6):1639-46).

Rats have also been used as models. Pulmonary Cryptococcus neoformans infection in rats results in a diffuse pneumonitis that resolves without dissemination or scarring except for the persistence of interstitial and subpleural granulomas that harbor viable cryptococci inside macrophages and epithelioid cells. Infected rats are asymptomatic but remain infected for as long as 18 months after inoculation with C. neoformans. Containment of infection is associated with granuloma formation that can be partially abrogated by glucocorticoid administration. Using this model, several features associated with persistent infection in the rat lung have been studied and can be used for monitoring the disease, including (i) localization of C. neoformans to discrete, well-organized granulomas; (ii) intracellular persistence of C. neoformans within macrophages and epithelioid cells; (iii) reduced inducible nitric oxide synthase expression by granulomas harboring C. neoformans; and (iv) reduced antibody responses to cryptococcal polysaccharide (Goldman et al., (2000) Infect. Immun. 68:832-838).

Another model based on weanling outbred rats has also been developed for the study of central nervous system infection with C. neoformans and used for testing an antifungal agent (Najvar et al., (1999) Antimicrob Agents Chemother 43:413-414). Weanling outbred rats were infected with C. neoformans by direct percranial puncture and inoculation into the cranium which led to lethality. Treatment with LY295337, a depsipeptide with antifungal activity, was effective in prolonging survival and reducing fungal counts in brain tissue.

Cortisone-treated rabbits have also been used in a new model for chronic cryptococcal meningitis. Normal rabbits soon recovered after intracisternal inoculation of C. neoformans, but cortisone-treated animals developed chronic progressive meningitis that was fatal in 2-12 weeks. Incidence and severity of infection was related to cortisone dose, not to inoculum size. The number of mononuclear cells that migrated into the subarachnoid spaces and cerebrospinal fluid of infected rabbits was strikingly reduced by cortisone treatment. Rabbits with cryptococcal meningitis were febrile; their high body temperature did not confer resistance to this infection. Potential applications of this model include study of the pathogenesis of cryptococcosis, and investigation of the immunobiology of the CNS in chronic meningitis (Perfect et al., (1980) Am J Pathol., 101:177-94). The cryptococcal meningitis model in corticosteroid-treated rabbits was used to assess the requirement for the phosphoribosylaminoimidazole gene (ADE2) for virulence of C. neoformans. A wild-type strain (H99), an ade2 auxotroph of H99 (M001), and a randomly selected prototrophic transformant of M001 (M001.1c) which had received the cloned ADE2 cDNA copy were inoculated intrathecally into immunosuppressed rabbits. While M001 was avirulent in the central nervous system model, virulence was completely restored to wild-type pathogenicity in the prototrophic transformant. (Perfect et al., (1993) Infect Immun 61:4446-51).

Recent findings that C. neoformans is a facultative intracellular pathogen indicate that this mode of infection may contribute to the persistence and latency of C. neoformans infection (Feldmesser et al., 2001, Trends in Microbiol. 9:273-278). Upon ingestion by a phagocytic cell, such as a macrophage or microglial cell, the yeast remains within the phagosome even when the phagosome is fused with a lysosome. Survival is associated with the accumulation of intracellular capsular polysaccharide and possibly synthesis of melanin. It is likely that gene expression during growth as a free-living saprophyte will be different than that if the fungal cell is located within a phagocytic cell of the host. Thus, expression of any given essential/virulence gene may be related to essentiality/virulence in either intracellular or extracellular environments, or both.

Accordingly, in yet another embodiment of the invention, the growth of conditional-expression C. neoformans mutants of the invention inside a phagocytic cell in the presence or absence of an inducer/repressor, such as tetracycline, can be monitored. Infected phagocytic cells can be observed directly with techniques such as intravital imaging (Farina et al., 1998, Cancer Res. 58:2528-2532) or observed in vitro after the phagocytic cell is removed from an infected animal. For example, in an in vivo phagocytosis assay, groups of mice can be infected with the mutants and after a period of time, the mice were killed and underwent bronchial lavage; alveolar macrophages were retrieved and allowed to adhere which are then fixed and stained to determine the number of phagocytosed C. neoformans cells. Also, the rate of yeast cell budding inside a cell and the number of yeast cells per phagosome can be determined.

In another embodiment, in vitro phagocytosis assays can be performed with murine macrophage cell lines, primary peritoneal and alveolar macropahages. The mutant yeast cells can be used to infect phagocytic cells in a tissue culture model in the presence or absence of an inducer/repressor. The number of mutant cells inside macrophages can be determined by lysing the macrophages and plating the cell supernatant and lysates in the appropriate plate medium for counting. Examples of animal models and tissue culture models include but are not limited to those described in Fries et al., 2001, J. Clin. Invest. 108:1639-1648; Goldman et al., 1998, Proc. Natl. Acad. Sci. 95:14967-14972; Lee et al., 1995, Lab. Invest. 73:871-879; and Luberto et al., 2001, Genes Dev. 15:201-212, which are incorporated herein by reference in their entireties.

5.4.4 Rational Design of Binding Compounds

Compounds identified via assays such as those described herein can be useful, for example, for inhibiting the growth of the infectious agent and/or ameliorating the symptoms of an infection. Compounds can include, but are not limited to, other cellular proteins. Binding compounds can also include, but are not limited to, peptides such as, for example, soluble peptides, comprising, for example, extracellular portions of target gene product transmembrane receptors, and members of random peptide libraries (see, e.g., Lam et al., 1991, Nature 354:82-84; Houghten et al., 1991, Nature 354:84-86) made of D-and/or L-configuration amino acids, rationally-designed antipeptide peptides, (see e.g., Hurby et al., Application of Synthetic Peptides: Antisense Peptides, “In Synthetic Peptides, A User's Guide”, W. H. Freeman, NY (1992), pp. 289-307), antibodies (including, but not limited to polyclonal, monoclonal, human, humanized, anti-idiotypic, chimeric or single chain antibodies, and FAb, F(ab′)₂ and FAb expression library fragments, and epitope-binding fragments thereof), and small organic or inorganic molecules. In the case of receptor-type target molecules, such compounds can include organic molecules (e.g., peptidomimetics) that bind to the ECD and either mimic the activity triggered by the natural ligand (i.e., agonists); as well as peptides, antibodies or fragments thereof, and other organic compounds that mimic the ECD (or a portion thereof) and bind to a “neutralize” natural ligand.

Computer modeling and searching technologies permit identification of compounds, or the improvement of already identified compounds, that can modulate target gene expression or activity. Having identified such a compound or composition, the active sites or regions are preferably identified. In the case of compounds affecting receptor molecules, such active sites might typically be ligand binding sites, such as the interaction domains of ligand with receptor itself. The active site is identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods are used to find the active site by finding where on the factor the complexed ligand is found.

The three-dimensional geometric structure of the active site is then preferably determined. This is done by known methods, including X-ray crystallography, which determines a complete molecular structure. Solid or liquid phase NMR is also used to determine certain intra-molecular distances within the active site and/or in the ligand binding complex. Other experimental methods of structure determination known to those of skill in the art, are also used to obtain partial or complete geometric structures. The geometric structures are measured with a complexed ligand, natural or artificial, which increases the accuracy of the active site structure determined. Methods of computer based numerical modeling are used to complete the structure (e.g., in embodiments wherein an incomplete or insufficiently accurate structure is determined) or to improve its accuracy.

Finally, having determined the structure of the active site, either experimentally, by modeling, or by a combination, candidate modulating compounds are identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential target or pathway gene product modulating compounds.

In general, the method is based on determining the three-dimensional structure of the polypeptide encoded by each essential gene, e.g., using X-ray crystallography or NMR, and using the coordinates of the determined structure in computer-assisted modeling programs to identify compounds that bind to and/or modulate the activity or expression level of encoded polypeptide. Thus, the method employs three basic steps: 1) the generation of high-purity crystals of the encoded recombinant (or endogenous) polypeptide for analysis; 2) determination of the three-dimensional structure of the polypeptide; and, 3) the use of computer-assisted “docking” programs to analyze the molecular interaction of compound structure and the polypeptide (i.e., drug screening).

General methods for performing each of the above steps are described below and are also well known to those of skill in the art. Any method known to those of skill in the art, including those described herein, may be employed for generating the three-dimensional structure for each identified essential gene product and its use in the drug-screening assays.

The products of the C. neoformans essential genes identified herein are used as molecular targets for rational drug design. In one embodiment, the three-dimensional structure of the product of the essential gene is determined using X-ray crystallography and the resulting crystallographic data are used in in silico drug screening assays to identify agents that are capable of binding to and modulating the amount or activity of the essential gene product.

Under special conditions, molecules condense from solution into a highly-ordered crystalline lattice, which is defined by a unit cell, the smallest repeating volume of the crystalline array. The contents of such a cell can interact with and diffract certain electromagnetic and particle waves (e.g., X-rays, neutron beams, electron beams etc.). Due to the symmetry of the lattice, the diffracted waves interact to create a diffraction pattern. By measuring the diffraction pattern, crystallographers attempt to reconstruct the three-dimensional structure of the atoms in the crystal.

A crystal lattice is defined by the symmetry of its unit cell and any structural motifs the unit cell contains. For example, there are 230 possible symmetry groups for an arbitrary crystal lattice, while the unit cell of the crystal lattice group may have an arbitrary dimension that depends on the molecules making up the lattice. Biological macromolecules, however, have asymmetric centers and are limited to 65 of the 230 symmetry groups. See Cantor et al., Biophysical Chemistry, Vol. III, W. H. Freeman & Company (1980), which is incorporated herein by reference in its entirety.

A crystal lattice interacts with electromagnetic or particle waves, such as X-rays or electron beams respectively, that have a wavelength with the same order of magnitude as the spacing between atoms in the unit cell. The diffracted waves are measured as an array of spots on a detection surface positioned adjacent to the crystal. Each spot has a three-dimensional position, hkl, and an intensity, I (hkl), both of which are used to reconstruct the three-dimensional electron density of the crystal with the so-called Electron Density Equation. The Electron Density Equation states that the three-dimensional electron density of the unit cell is the Fourier transform of the structure factors. Thus, in theory, if the structure factors are known for a sufficient number of spots in the detection space, then the three-dimensional electron density of the unit cell could be calculated using the Electron Density Equation.

Another aspect of the present invention comprises a method of using a crystal of the present invention and/or a dataset comprising the three-dimensional coordinates obtained from the crystal in a drug-screening assay. The present invention further provides the novel agents (modulators or drugs) that are identified by the method of the present invention, along with the method of using agents (modulators or drugs) identified by a method of the present invention, for inhibiting the activity of or modulating the amount of an essential gene product.

This method of drug screening relies on structure based drug design. In this case, the three dimensional structure of product of the essential gene is determined and potential agonists and/or potential antagonists are designed with the aid of computer modeling (Bugg et al., Scientific American, Dec.:92-98 (1993); West et al., TIPS, 16:67-74 (1995); Dunbrack et al., Folding & Design, 2:27-42 (1997)). However, heretofore the three-dimensional structure of the product of the essential genes identified herein has remained unknown. Therefore, there is a need for obtaining a crystal of these gene products with sufficient quality to allow high quality crystallographic data to be obtained. Furthermore there is a need for the determination of the three-dimensional structure of such crystals. Finally, there is a need for procedures for related structural based drug design predicated on such crystallographic data.

Computer analysis may be performed with one or more of the computer programs including: QUANTA, CHARMM, FlexX, INSIGHT, SYBYL, MACROMODEL and ICM (Dunbrack et al., Folding & Design, 2:27-42 (1997)). In a further embodiment of this aspect of the invention, an initial drug-screening assay is performed using the three-dimensional structure so obtained, preferably along with a docking computer program. Such computer modeling can be performed with one or more Docking programs such as DOC, FlexX, GRAM and AUTO DOCK (Dunbrack et al., Folding & Design, 2:27-42 (1997)).

It should be understood that for each drug screening assay provided herein, a number of iterative cycles of any or all of the steps may be performed to optimize the selection. The drug screening assays of the present invention may use any of a number of means for determining the interaction between an agent or drug and an C. neoformans essential gene product.

In one such assay, a drug can be specifically designed to bind to an essential gene of the present invention through NMR based methodology, (Shuker et al., Science 274:1531-1534 (1996) hereby incorporated by reference herein in its entirety). NMR Spectroscopy and Structure Calculations: NMR spectra were recorded at 23° C. using Varian Unity Plus 500 and unity 600 spectrometers, each equipped with a pulsed-field gradient triple resonance probe as analyzed as described in Bagby et al., (Cell 82:857-867 (1995)) hereby incorporated by reference in its entirely. Sequential resonance assignments of backbone ¹H, ¹⁵N, and ¹³C atoms were made using a combination of triple resonance experiments similar to those previously described (Bagby et al., Biochemistry, 33:2409-2421 (1994)), except with enhanced sensitivity (Muhandiram and Kay, J. Magn. Reson., 103: 203-216 (1994)) and minimal H₂O saturation (Kay et al., J. Magn. Reson., 109:129-133 (1994)). Side chain ¹H and ¹³C assignments were made using HCCH-TOCSY (Bax et al., J. Magn. Reson., 87:620-627 (1990)) experiments with mixing times of 8 ms and 16 ms in solution and were not included in structure calculations. Nuclear Overhauser effect (NOE) cross peaks in two-dimensional ¹H-¹H NOE spectroscopy (NOESY), three-dimensional ¹⁵N-edited NOESY-HSQC (Zhang et al., J. Biomol, NMR, 4:845-858 (1994)) and three-dimensional simultaneous acquisition ¹⁵N/¹³C-edited NOE (Pascal et al., J. Magn. Reson., 103:197-201 (1994)) spectra were obtained with 100 ms NOE mixing times. Standard pseudo-atom distance corrections (Wuthrich et al., J. Mol. Biol., 169:949-961 (1983)) were incorporated to account for center averaging. An additional 0.5 D was added to the upper limits for distances involving methyl groups (Wagner et al., J. Mol. Biol., 196:611-639 (1987); Clore et al., Biochemistry, 26:8012-8023 (1987)).

The structures are calculated using a simulated annealing protocol (Nilges et al., In computational Aspects of the Study of Biological Macromolecules by Nuclear Magnetic Resonance Spectroscopy, J. C. Hoch, F. M. Poulsen, and C. Redfield, eds., New York: Plenum Press, pp. 451-455 (1991) within X-PLOR (Brunger, X-PLOR Manual, Version 3.1, New Haven, Conn.: Department of Molecular Biophysics and Biochemistry, Yale University (1993) using the previously described strategy (Bagby et al., Structure, 2:107-122 (1994)). Interhelical anges were calculated using an in-house program written by K. Yap. Accessible surface areas were calculated using the program Naccess, available from Prof. J. Thornton, University College, London.

Any method known to those of skill in the art, including those set forth below, may be employed to prepare high-purity crystals. For example, crystals of the product of the identified essential gene can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used. Exemplified below is the hanging-drop vapor diffusion procedure. Hanging drops of an essential gene product (2.5 μl, 10 mg/ml) in 20 mM Tris, pH 8.0, 100 mM NaCl are mixed with an equal amount of reservoir buffer containing 2.7-3.2 M sodium formate and 100 mM Tris buffer, pH 8.0, and kept at 4° C. Crystal showers may appear after 1-2 days with large single crystals growing to full size (0.3×0.3×0.15 mm³) within 2-3 weeks. Crystals are harvested in 3.5 M sodium formate and 100 mM Tris buffer, pH 8.0 and cryoprotected in 3.5 M sodium formate, 100 mM Tris buffer, pH 8.0, 10% (w/v) sucrose, and 10% (v/v) ethylene glycol before flash freezing in liquid propane. Once a crystal of the present invention is grown, X-ray diffraction data can be collected.

Therefore, any person with skill in the art of protein crystallization having the present teachings and without undue experimentation could crystallize a large number of alternative forms of the essential gene products from a variety of different organisms, or polypeptides having conservative substitutions in their amino acid sequence.

Once the three-dimensional structure of a crystal comprising an essential gene product is determined, a potential modulator of its activity can be examined through the use of computer modeling using a docking program such as GRAM, DOCK, FlexX or AUTODOCK (Dunbrack et al., 1997, supra), to identify potential modulators. This procedure can include computer fitting of potential modulators to the polypeptide or fragments thereof to ascertain how well the shape and the chemical structure of the potential modulator will bind. Computer programs are employed to estimate the attraction, repulsion, and steric hindrance of the two binding partners (e.g., the essential gene product and a potential modulator). Generally the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as well with other proteins. This will minimize potential side-effects due to unwanted interactions with other proteins.

Compound and compound analogs can be systematically modified by computer modeling programs until one or more promising potential analogs is identified. In addition systematic modification of selected analogs can then be systematically modified by computer modeling programs until one or more potential analogs are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). Alternatively a potential modulator could be obtained by initially screening a random peptide library produced by recombinant bacteriophage for example, (Scott and Smith, Science, 249:386-390 (1990); Cwirla et al., Proc. Natl. Acad. Sci., 87:6378-6382 (1990); Devlin et al., Science, 249:404-406 (1990)). A peptide selected in this manner would then be systematically modified by computer modeling programs as described above.

Alternatively, these methods are used to identify improved modulating compounds from an already known modulating compound or ligand. The composition of the known compound is modified and the structural effects of modification are determined using the experimental and computer modeling methods described above applied to the new composition. The altered structure is then compared to the active site structure of the compound to determine if an improved fit or interaction results. In this manner systematic variations in composition, such as by varying side groups, are quickly evaluated to obtain modified modulating compounds or ligands of improved specificity or activity.

Further experimental and computer modeling methods useful to identify modulating compounds based upon identification of the active sites of target or pathway gene or gene products and related transduction and transcription factors are apparent to those of skill in the art.

There are a number of articles that review the art of computer modeling of drugs that interact with specific proteins, including the following: Rotivinen et al., 1988, Acta Pharmaceutical Fennica 97:159-166; Ripka, (Jun. 16, 1988), New Scientist 54-57; McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 1-162; and, with respect to a model receptor for nucleic acid components, Askew et al., 1989, J Am. Chem. Soc. 111:1082-1090.

Although generally described above with reference to design and generation of compounds which could alter binding, one could also screen libraries of known compounds, including natural products or synthetic chemicals, as well as other biologically active materials, including proteins, for compounds which are inhibitors or activators.

### 5.5 Transcriptional Profiling

5.5.1 Analysis of Gene Expression

Gene expression profiling techniques are important tools for the identification of suitable biochemical targets, as well as for the determination of the mode of action of known compounds. Large scale sequencing of the C. neoformans genome and development of nucleic acid arrays or microarrays incorporating this information, will enable genome-wide gene expression analyses to be carried out with this diploid pathogenic fungus. Technologies available for making and using very large arrays of nucleic acids are well known in the art, see, for example, Schena, M., “Microarray Biochip Technology,” 2000, Eaton Publishing, MA, in particular Chapters 1 and 2, and also U.S. Pat. No. 5,143,854 and PCT Publication Nos. WO 90/15070 and 92/10092, each of which is hereby incorporated by reference. See, also, U.S. Pat. Nos. 5,412,087, 5,445,934 and 5,744,305; Grigorenko, “DNA Arrays: Technologies and Experimental Strategies”, CRC Press, 2001; and Rampal, “DNA Arrays: Methods and Protocols”, Humana Press 2001, each of which is hereby incorporated by reference. In particular embodiments, the nucleic acid arrays used for profiling can comprise one or more of the nucleic acid molecules of the essential genes of the invention, preferably DNA comprising the entire or a portion of SEQ ID NO. 2001-2361, or DNA comprising a nucleotide sequence that encode the entire or a portion of SEQ ID NO: 3001-3361. The present invention provides methods for obtaining the transcriptional response profiles for both essential and virulence/pathogenicity genes of C. neoformans. Conditional expression of essential genes serves to delineate, for example, regulatory interactions valuable for the design of drug screening programs focused upon C. neoformans.

In an embodiment of the present invention, the conditional-expression C. neoformans mutant strain collection is used for the analysis of expression of essential genes within this pathogen. One particularly powerful application of such a strain collection involves the construction of a comprehensive transcriptional profile database for the entire essential gene set or a desired subset of essential genes within a pathogen. Such a database is used to compare the response profile characteristic of lead antimycotic compounds with the profile obtained with new anti-fungal compounds to distinguish those with similar from those with distinct modes of action. Matching (or even partially overlapping) the transcriptional response profiles determined after treatment of the strain with the lead compound with that obtained with a particular essential target gene under repressing conditions, is used to identity the target and possible mode of action of the drug. See, for example, U.S. Pat. No. 6,004,755 which disclose methods for quantitative gene expression analysis with nucleic acid arrays; and U.S. Pat. No. 6,263,287 which discloses methods and computer systems for the analysis and manipulation of gene expression data, which are incorporated herein by reference in their entirety.

Gene expression analysis of essential genes also permits the biological function and regulation of those genes to be examined within the pathogen, and this information is incorporated within a drug screening program. For example, transcriptional profiling of essential drug targets in C. neoformans permits the identification of novel drug targets which participate in the same cellular process or pathway uncovered for the existing drug target and which could not otherwise be identified without direct experimentation within the pathogen. These include genes not only unique to the pathogen but also broad-range gene classes possessing a distinct function or subject to different regulation in the pathogen. Furthermore, pathogen-specific pathways may be uncovered and exploited for the first time. See, for example, U.S. Pat. No. 6,340,565 (which is incorporated herein by reference in their entirety), which discloses an gene expression array-based approach to the systematic analysis of relationships between expression patterns of genes as affected by the activities of other genes, which can be adapted to investigate the effect of pathogen gene expression in the presence of various compounds identified by the methods of the invention.

In another aspect of the present invention, the gene expression profile of conditional-expression C. neoformans mutant strains under nonrepressing or induced conditions is established to evaluate the overexpression response profile for one or more drug targets. For example, overexpression of genes functioning in signal transduction pathways often display unregulated activation of the pathway under such conditions. Moreover, several signaling pathways have been demonstrated to function in the pathogenesis process. Transcriptional response profiles generated by overexpressing conditional-expression C. neoformans mutant strains provide information concerning the set of genes regulated by such pathways; any of which may potentially serve an essential role in pathogenesis and therefore representing promising drug targets. Furthermore, analysis of the expression profile may reveal one or more genes whose expression is critical to the subsequent expression of an entire regulatory cascade. Accordingly, these genes are particularly important targets for drug discovery and mutants carrying the corresponding modified allelic pair of genes form the basis of a mechanism-of-action based screening assays. Presently such an approach is not possible. Current drug discovery practices result in an exceedingly large number of “candidate” compounds and little understanding of their mode of action. A transcriptional response database comprising both gene shut-off and overexpression profiles generated using the conditional-expression C. neoformans mutant strain collection offers a solution to this drug discovery bottleneck by 1) determining the transcriptional response or profile resulting from an antifungal's inhibition of a wild type strain, and 2) comparing this response to the transcriptional profiles resulting from inactivation or overexpression of drug targets comprising the conditional-expression C. neoformans mutant strain collection.

Matching or significantly correlating transcriptional profiles resulting from both genetic alteration of a drug target and chemical/compound inhibition of wild type cells provides evidence linking the compound to its cellular drug target and suggests its mechanism of action.

Accordingly, the invention provides a method for evaluating a compound against a target gene product encoded by a nucleotide sequence comprising one of SEQ ID NOs: 2001-2361, as well as the gene product encoded by genomic SEQ ID NOs: 1-361 and 1001-1361, as expressed by C. neoformans, said method comprising the steps of (a) contacting wild type diploid fungal cells or control cells with the compound and generating a first transcription profile; (b) determining the transcription profile of mutant fungal cells, such as a conditional-expression C. neoformans mutant strain, which have been cultured under conditions wherein the second allele of the target gene is substantially underexpressed, not expressed or overexpressed and generating a second transcription profile for the cultured cells; and comparing the first transcription profile with the second transcription profile to identify similarities in the profiles. For comparisons, similarities of profiles can be expressed as an indicator value; and the higher the indicator value, the more desirable is the compound.

5.5.2 Identification of Secondary Targets

Methods are described herein for the identification of secondary targets. “Secondary target,” as used herein, refers to a gene whose gene product exhibits the ability to interact with target gene products involved in the growth and/or survival of an organism (i.e., target essential gene products), under a set of defined conditions, or in the pathogenic mechanism of the organism, (i.e., target virulence gene products) during infection of a host.

Any method suitable for detecting protein-protein interactions can be employed for identifying secondary target gene products by identifying interactions between gene products and target gene products. Such known gene products can be cellular or extracellular proteins. Those gene products which interact with such known gene products represent secondary target gene products and the genes which encode them represent secondary targets. Well known techniques such as those described in Golemis and Serebriiskii, “Protein-protein interactions: A molecular cloning manual”, Cold Spring Harbor Laboratory Press, 2002, which is incorporated herein by reference, can be used.

Among the traditional methods employed are co-immunoprecipitation, crosslinking and co-purification through gradients or chromatographic columns. Utilizing procedures such as these allows for the identification of secondary target gene products. Once identified, a secondary target gene product is used, in conjunction with standard techniques, to identify its corresponding secondary target. For example, at least a portion of the amino acid sequence of the secondary target gene product is ascertained using techniques well known to those of skill in the art, such as via the Edman degradation technique (see, e.g., Creighton, 1983, “Proteins: Structures and Molecular Principles,” W. H. Freeman & Co., N.Y., pp.34-49). The amino acid sequence obtained can be used as a guide for the generation of oligonucleotide mixtures that can be used to screen for secondary target gene sequences. Screening can be accomplished, for example, by standard hybridization or PCR techniques. Techniques for the generation of oligonucleotide mixtures and for screening are well-known. (See, e.g., Ausubel, supra., and PCR Protocols: A Guide to Methods and Applications, 1990, Innis, M. et al., eds. Academic Press, Inc., New York).

Additionally, methods are employed which result in the simultaneous identification of secondary targets which encode proteins interacting with a protein involved in the growth and/or survival of an organism under a set of defined conditions, or in the pathogenic mechanism of the organism during infection of a host. These methods include, for example, probing expression libraries with labeled primary target gene protein known or suggested to be involved in or critical to these mechanisms, using this protein in a manner similar to the well known technique of antibody probing of ëgt11 phage libraries.

One method which detects protein interactions in vivo, the two-hybrid system, is described in detail for illustration purposes only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to a known protein, in this case, a protein known to be involved in growth of the organism, or in pathogenicity, and the other consists of the activator protein's activation domain fused to an unknown protein that is encoded by a cDNA which has been recombined into this plasmid as part of a cDNA library. The plasmids are transformed into a strain of the yeast Saccharomyces cerevisiae that contains a reporter gene (e.g., lacZ) whose regulatory region contains the transcription activator's binding sites. Either hybrid protein alone cannot activate transcription of the reporter gene, the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology is used to screen activation domain libraries for proteins that interact with a known “bait” gene product. By way of example, and not by way of limitation, target essential gene products and target virulence gene products are used as the bait gene products. Total genomic or cDNA sequences encoding the target essential gene product, target virulence gene product, or portions thereof, are fused to the DNA encoding an activation domain. This library and a plasmid encoding a hybrid of the bait gene product fused to the DNA-binding domain are cotransformed into a yeast reporter strain, and the resulting transformants are screened for those that express the reporter gene. For example, and not by way of limitation, the bait gene is cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait gene product are to be detected is made using methods routinely practiced in the art. According to the particular system described herein, for example, the cDNA fragments are inserted into a vector such that they are translationally fused to the activation domain of GAL4. This library is co-transformed along with the bait gene-GAL4 fusion plasmid into a yeast strain which contains a lacZ gene driven by a promoter which contains GAL4 activation sequence. A cDNA encoded protein, fused to GAL4 activation domain, that interacts with bait gene product reconstitutes an active GAL4 protein and thereby drive expression of the lacZ gene. Colonies which express lacZ are detected by their blue color in the presence of X-gal. The cDNA can then be purified from these strains, and used to produce and isolate the bait gene-interacting protein using techniques routinely practiced in the art.

Once a secondary target has been identified and isolated, it is further characterized and used in drug discovery by the methods of the invention.

5.5.3 Use of Gene Expression Arrays

To carry out profiling, gene expression arrays and microarrays can be employed. Gene expression microarrays are high density arrays of DNA samples deposited at specific, preferably spatially addressible locations on a solid substrate, such as a glass surface, silicon, nylon membrane, or the like. Such arrays are used by researchers to quantify relative gene expression under different conditions. An example of this technology is found in U.S. Pat. No. 5,807,522, which is hereby incorporated by reference.

It is possible to study the expression of substantially all of the genes in the genome of a particular microbial organism using a single array. For example, the arrays may consist of 12×24 cm nylon filters containing PCR products corresponding to ORFs from C. neoformans. 10 ngs of each PCR product are spotted every 1.5 mm on the filter. Single stranded labeled cDNAs are prepared for hybridization to the array (no second strand synthesis or amplification step is done) and placed in contact with the filter. Thus the labeled cDNAs are of “antisense” orientation. Quantitative analysis is done using a phosphorimager.

Hybridization of cDNA made from a sample of total cell mRNA to such an array followed by detection of binding by one or more of various techniques known to those in the art provides a signal at each location on the array to which cDNA hybridized. The intensity of the hybridization signal obtained at each location in the array thus reflects the amount of mRNA for that specific gene that was present in the sample. Comparing the results obtained for mRNA isolated from cells grown under different conditions thus allows for a comparison of the relative amount of expression of each individual gene during growth under the different conditions.

Gene expression arrays are used to analyze the total mRNA expression pattern at various time points after reduction in the level or activity of a gene product required for fungal proliferation, virulence or pathogenicity. Reduction of the level or activity of the gene product is accomplished by growing a conditional-expression C. neoformans mutant strain under conditions in which the product of the nucleic acid linked to the regulatable promoter is rate limiting for fungal growth, survival, proliferation, virulence or pathogenicity or by contacting the cells with an agent which reduces the level or activity of the target gene product. Analysis of the expression pattern indicated by hybridization to the array provides information on other genes whose expression is influenced by reduction in the level or activity of the gene product. For example, levels of other mRNAs may be observed to increase, decrease or stay the same following reduction in the level or activity of the gene product required for growth, survival, proliferation, virulence or pathogenicity. Thus, the mRNA expression pattern observed following reduction in the level or activity of a gene product required for growth, survival, proliferation, virulence or pathogenicity identifies other nucleic acids required for growth, survival, proliferation, virulence or pathogenicity. In addition, the mRNA expression patterns observed when the fungi are exposed to candidate drug compounds or known antibiotics are compared to those observed when the level or activity of a gene product required for fungal growth, survival, proliferation, virulence or pathogenicity is reduced. If the mRNA expression pattern observed with the candidate drug compound is similar to that observed when the level of the gene product is reduced, the drug compound is a promising therapeutic candidate. Thus, the assay is useful in assisting in the selection of promising candidate drug compounds for use in drug development.

In cases where the source of nucleic acid deposited on the array and the source of the nucleic acid being hybridized to the array are from two different microorganisms, gene expression identify homologous genes in the two microorganisms.

### 5.6 Proteomics Assays

In another embodiment of the present invention, and in much the same way that the conditional-expression C. neoformans mutant strain collection enables transcriptional profiling within a pathogen, a conditional-expression C. neoformans mutant strain collection provides an invaluable resource for the analysis of the expressed protein complement of a genome. By evaluating the overall protein expression by members of a conditional-expression C. neoformans mutant strain collection under repressing and non-repressing growth conditions, a correlation between the pattern of protein expression of a cell can be made with the non-expression or the level of expression of an essential gene. A plurality of protein expression patterns will be generated for a conditional-expression C. neoformans mutant strain when the strain is cultured under different conditions and different levels of expression of one of the modified allele. The set of proteins analyzed comprises one or more proteins comprising an amino acid sequence selected from the group consisting of the amino acid sequences of SEQ ID NO: 3001 to 3361. A preferred mutant strain collection for performing such analysis is a conditional-expression mutant strain collection.

Evaluation of the full complement of proteins expressed within a cell depends upon definitive identification of all protein species detectable on two-dimensional polyacrylamide gels or by other separation techniques. However, a significant fraction of these proteins are of lower abundance and fall below the threshold level required for positive identification by peptide sequencing or mass spectrometry. Nevertheless, these Aorphan@ proteins are detectable using an analysis of protein expression by individual conditional-expression C. neoformans mutant strains. Conditional expression of low abundance gene products facilitates their positive identification by comparing protein profiles of conditional-expression C. neoformans mutant strains under repressing versus nonrepressing or overexpression conditions. In some cases, a more complex protein profile results because of changes of steady state levels for multiple proteins, which is caused indirectly by manipulating the low abundance gene in question. Overexpression of individual targets within the conditional-expression C. neoformans mutant strain collection can also directly aid orphan protein identification by providing sufficient material for peptide sequencing or mass spectrometry.

In yet another embodiment, defined genetic mutations can be constructed to create strains exhibiting protein expression profiles comparable to those observed upon treatment of the strain with a previously uncharacterized compound. In this way, it is possible to distinguish between antimycotic compounds that act on multiple targets in a complicated manner from other potential lead compounds that act on unique fungal-specific targets and whose mode of action can be determined. Matching the pattern of protein expression determined after treatment of a strain with a lead compound with that obtained with a particular essential target gene under repressing conditions, can be used to identity the target and possible mode of action of the drug.

Accordingly, the invention provides a pattern of expression of a set of proteins in a mutant strain as determined by methods well known in the art for establishing a protein expression pattern. The pattern typically takes the form of computer data comprising the identities of the proteins, and for each protein, an indicator for the presence or absence of the proteins, and/or a level of expression expressed in absolute value or in a value relative to a standard. Two-dimensional gel electrophoresis is well known in the art for generating and qualitatively analyzing a pattern of expression of a set of proteins. Mass spectroscopy is a highly accurate analytical tool for determining molecular weights and identifying chemical structures. Proteins and peptides have been studied by matrix-assisted laser desorption mass spectroscopy and electrospray ionization mass spectroscopy. See, for example, Chait, Brian T. and Kent, Stephen B. H., 1992, “Weighing Naked Proteins: Practical, High-Accuracy Mass Measurement of Peptides and Proteins”, Science, 257:1885-1894, and U.S. Pat. No. 6,391,649, which are incorporated by reference herein. Matrix-assisted laser desorption time-of-flight mass spectrometers are described in U.S. Pat. Nos. 5,045,694 and 5,453,247, which are incorporated by reference herein. Electrospray ionization mass spectrometers are described in U.S. Pat. No. 5,245,186 and U.S. Pat. No. 4,977,320, which are also incorporated by reference herein. For a detailed description of methods and protocols that can be used to analyze the protein expression patterns, see for example, Link, “2-D Proteome Analysis Protocols”, Humana Press 1988.

In various embodiments, the present invention provides a method of quantitative analysis of the expressed protein complement of a diploid pathogenic fungal cell: a first protein expression profile is developed for a control diploid pathogenic fungus, which has two, unmodified alleles for the target gene. Mutants of the control strain, in which one allele of the target gene is inactivated, for example, in a conditional-expression C. neoformans mutant strain, by insertion by or replacement with a disruption cassette, is generated. The other allele is modified such that expression of that second allele is under the control of a heterologous regulated promoter. A second protein expression profile is developed for this mutant fungus, under conditions where the second allele is substantially overexpressed as compared to the expression of the two alleles of the gene in the control strain. Similarly, if desired, a third protein expression profile is developed, under conditions where the second allele is substantially underexpressed as compared to the expression of the two alleles of the gene in the control strain. The first protein expression profile is then compared with the second expression profile, and if applicable, a third protein expression profile to identify an expressed protein detected at a higher level in the second profile, and if applicable, at a lower level in the third profile, as compared to the level in first profile.

Accordingly, the invention provides a method for evaluating a compound against a target gene product encoded by a nucleotide sequence comprising one of SEQ ID NOs: 2001-2361, as well as the gene product encoded by genomic SEQ ID NOs: 1-361 and 1001-1361, as expressed by C. neoformans, said method comprising the steps of (a) contacting wild type diploid fungal cells or control cells with the compound and generating a first protein expression profile; (b) determining the protein expression profile of mutant diploid fungal cells, such as a conditional-expression C. neoformans mutant strain, which have been cultured under conditions wherein the second allele of the target gene is substantially underexpressed, not expressed or overexpressed and generating a second protein expression profile for the cultured cells; and comparing the first protein expression profile with the second protein expression profile to identify similarities in the profiles. For comparisons, similarities of profiles can be expressed as an indicator value; and the higher the indicator value, the more desirable is the compound.

### 5.7 Pharmaceutical Compositions And Uses Thereof

Compounds including nucleic acid molecules that are identified by the methods of the invention as described herein can be administered to a subject at therapeutically effective doses to treat or prevent infections by a pathogenic organism, such as C. neoformans. Depending on the target, the compounds may also be useful for treatment of a non-infectious disease in a subject, such as but not limited to, cancer. C. neoformans infection enhances HIV expression in monocytic cells that may further impair host immunity and contribute to the acceleration of the course of HIV disease (Harrison et al., (1997) J Infect Dis. 176(2):485-491). Thus, prevention or treatment of C. neoformans infection may prolong life span of HIV infected patients from the disease itself and not just from infections by the opportunistic fungi.

In another embodiment, the compounds can be used to treat or prevent infection of domesticated animals by C. neoformans. In particular, household pets, e.g., cats, dogs, and birds, can get infected suggesting that these animals can be recipients of the compounds, see Barr et al., (2000) Aust Vet J 78(3):154-158. In one instance, a 12-year-old, FIV-positive, cat was presented with a history of sneezing and coughing during the previous seven months. On thoracic radiographs, a prominent bronchial pattern and three focal, opacified nodules were seen. Cytology of bronchoalveolar lavage fluid demonstrated spherical, capsulate, narrow-necked, budding yeasts within macrophages. Culture of the fluid yielded a heavy growth of C. neoformans var neoformans. The veterinary use of the compounds of the invention are contemplated.

A therapeutically effective dose refers to that amount of a compound (including nucleic acid molecules) sufficient to result in a healthful benefit in the treated subject. Typically, but not so limited, the compounds act by reducing the activity or level of a gene product encoded by a nucleic acid comprising a sequence selected from the group consisting of SEQ ID NOs: 2001-2361, as well as the gene product encoded by genomic SEQ ID NOs: 1-361 and 1001-1361, as expressed by C. neoformans. The subject to be treated can be a plant, a vertebrate, a mammal, an avian, or a human. These compounds can also be used for preventing or containing contamination of an object by C. neoformans, or used for preventing or inhibiting formation on a surface of a biofilm comprising C. neoformans. Biofilm comprising C. neoformans are found on surfaces of medical devices, such as but not limited to surgical tools, implanted devices, catheters and stents.

5.7.1 Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. A useful dosage can range from 0.001 mg/kg body weight to 10 mg/kg body weight.

5.7.2 Formulations and Use

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g, sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 6. EXAMPLES

The following experiments are carried out using the preferred Cryptococcus neoformans strain of H99, which is a well-characterized virulent clinical isolate of serotype A (e.g., see Nelson et al., (2001) Genetics 157:935-947).

### 6.1 Media

Suitable media for culturing C. neoformans include, but are not limited to, 1) YPD (1% yeast extract, 1% bacto-peptone, and 2% glucose); 2) RPMI 1640 (Sigma, St. Louis, Mo.); 3) low iron medium (Nyhus and Jacobson (1999) Infect. Immun 67:2357-2365) for analysis of casule formation; 4) yeast nitrogen base (YNB) pH 5.6 and pH 7.0; 5) RPMI MOPS pH 7.0 with and without added glucose (2%); 6) RPMI buffered with phosphate buffer to pH 7.0. (Some isolates yield poor growth in RPMI MOPS after 72 h. Tests indicated that YNB pH 5.6 was the best medium for 5-fluorocytosine but was unsuitable for ketoconazole (Petrou and Shanson (2000) J Antimicrob Chemother 46(5):815-818)); glucose salts/urea/basal medium (GSU); 7) Birdseed Agar (Niger seed agar); 8) Caffeic Acid Ferric Citrate Test Medium (CAFC; C. neoformans light brown pigmentation); 9) CN Screen Medium (C. neoformans black colonies); 10) Glycine Cycloheximide Phenol Red Agar (C. neoformans bright red); and 11) Inositol Urea Caffeic Acid Medium (inositol sole carbon source, urea sole nitrogen source; C. neoformans dark brown; others unpigmented). See, Atlas, In Handbook of Microbiological Media (Parks ed.), 1993, CRC Press, Inc., pp. 135, 178, 254, 401-402, 454 and 915, respectively for 7-11.

### 6.2 Isolation of Genomic DNA

Genomic DNA from C. neoformans can be isolated as described in Nelson et al., (2001) Genetics 157:935-947. The method is a modified version of glass bead yeast DNA extraction procedure of Fujimura and Sakuma (1993) Biotechniques 14:538-540. Briefly, C. neoformans cells are resuspended in 250 μl of lysis buffer (50 mm Tris, pH 7.5, 20 mM EDTA, 1% SDS) with 250 mg of glass beads (425-600 μM, Sigma), and the cells are disrupted by vortexing for 1 min at high speed followed by one min on ice six times. The mixture is incubated at 70° C. for 10 min, briefly vortexed, 100 μl of a 5 M KOAc solution and 75 μl of 5 M NaCl solution are added and the mixture is placed on ice for 20 min. The cooled mixture is then subjected to centrifugation at 15,300×g for 20 min, the supernatant transferred to a fresh tube and extracted with chloroform. The aqueous phase is separated by subjecting the sample to centrifugation at 15,300×g for 10 min. The DNA is separate from the aqueous phase by addition of 100 μl each of solutions of 30% polyethylene glycol and 1.5 M NaCl, and incubated on ice for 10 min. Genomic DNA was purified by subjecting the cooled mixture to high-speed centrifugation at 20,800×g for 10 min.

### 6.3 Promoter Replacement and Conditional Expression of An Essential Gene

The following example demonstrates that promoter replacement and conditional expression of an C. neoformans essential gene is achievable by homologous recombination using a linear promoter replacement cassette.

6.3.1 Preparation of the A Promoter Replacement Cassette

A gene that encodes a polypeptide product essential for growth of C. neoformans in a selective medium is used as an example. The promoter of the essential gene is replaced with a regulatable, heterologous promoter using a linear promoter replacement cassette. The promoter replacement cassette was designed to integrate into the genome by homologous recombination between regions of nucleotide sequence identity flanking the promoter of the essential gene. Proper integration of the cassette results in deletion of the promoter and introduction of the heterologous promoter, which is functional in C. neoformans. The cassette also contains a gene encoding a selectable marker, the nourseothricin acetyltransferase gene (nat1, McDade and Cox (2001) Med. Mycol. 39:151-154), for selection and easy identification of integrative transformants. The cassette can be assembled using methods well known in the art.

6.3.2 Transformation of C. neoformans

Several methods for transforming C. neoformans known in the art can be used.

A transformation system using resistance to the antibiotic cycloheximide as a dominant selectable marker has been developed (Varma et al., (2000) Yeast 16:1397-1403). Toffaletti et al., [(1993) J Bacteriol 175(5):1405-11] and Davidson et al., [(2000) Fungal Genet Biol 29:38-48] describe two transformation systems for the disruption of genes by homologous recombination. The first is based on electroporation of all serotypes (A & D experimentally) which results in identifying recombinants/integrants at frequencies of 10⁻³ to 10⁻⁵. The second is a biolistic transformation method that results in identifying recombinants/integrants at frequencies between 2% -50% in serotype A strains and 1% -4% in serotype D strains.

A modified version of the method described in Davidson et al., (2000) Fungal Genet. Biol. 29:38-48 is described hereinbelow:

C. neoformans culture is grown overnight at 30° C. with shaking in YPD medium and the cells are pelleted by centrifugation. The cells are resuspended in 10 ml of YPD medium and approximately 200 μl of the cell suspension is spread onto YPD pates supplemented with 400 μg/ml nourseothricin (or hygromycin; and may optionally include bacterial antibiotics, such as ampicillin and/or streptomycin at 100 μg/ml each. The plates are allowed to dry. DNA samples are prepared by combining 600 μg of gold microcarrier beads (0.6 μm, Bio-Rad) with 1-4 μg of plasmid DNA comprising the nat1 selectable marker and promoter replacement cassette or flanking homology for gene disruption, 10 μl of 2.5. M CaCl₂ and 2 μl of 1 M spermidine. The mixture is incubated for 5 minutes at room temperature, subjected to centrifugation, washed with 1.5 mL of 100% ethanol, the beads are resuspended in 12 μl of 100% ethanol and placed onto microcarrier disks (Bio-Rad). The plates are then bombarded with the DNA-bound gold microcarrier beads in a biolistic transformation apparatus (Bio-Rad) at a helium gas pressure of approximately 1350 psi under vacuum in 29 in. of mercury. The plates are then incubated at 30° C. and nourseothricin-resistant transformants are isolated.

### 6.4 Isolation of a Strain Comprising a Heterologous Promoter

Nourseothricin-resistant transformants are streaked for isolated single colonies and screened for those colonies capable of growth in the selective medium. Oligonucleotide primers based on the targeted gene are used in pairwise combination to amplify a nucleic acid molecule comprising a nucleotide sequence spanning the junction region for each transformant. Proper integration of the cassette produces an amplification product of a certain expected size whereas amplification of the endogenous essential gene results in the production of an amplification of a different size.

### 6.5 Identification of a Drug Target or Target Pathway

A target pathway is a genetic or biochemical pathway wherein one or more of the components of the pathway (e.g., enzymes, signaling molecules, etc) is a drug target as determined by the methods of the invention.

6.5.1 Preparation of Stocks of Conditional-expression C. neoformans Mutant Strains for Assay

To provide a consistent source of cells to screen, frozen stocks of host conditional-expression C. neoformans mutant strains are prepared using standard microbiological techniques. For example, a single clone of the microorganism can be isolated by streaking out a sample of the original stock onto an agar plate containing nutrients for cell growth and an antibiotic for which the conditional-expression C. neoformans mutant strain contains a gene which confers resistance. After overnight growth an isolated colony is picked from the plate with a sterile needle and transferred to an appropriate liquid growth medium containing the antibiotic to which the conditional-expression C. neoformans mutant strain is resistant. The cells are incubated under appropriate growth conditions to yield a culture in exponential growth. Cells are frozen using standard techniques.

6.5.2 Growth of Conditional-expression C. neoformans Mutant Strains for Use in the Assay

Prior to performing an assay, a stock vial is removed from the freezer, rapidly thawed and a loop of culture is streaked out on an agar plate containing nutrients for cell growth and an antibiotic for which the conditional-expression C. neoformans mutant strain contains a gene which confers resistance. After overnight growth, randomly chosen, isolated colonies are transferred from the plate (sterile inoculum loop) to a sterile tube containing medium containing the antibiotic to which the conditional-expression C. neoformans mutant strain contains a gene which confers resistance. After vigorous mixing to form a homogeneous cell suspension, the optical density of the suspension is measured and if necessary an aliquot of the suspension is diluted into a second tube of medium plus antibiotic. The culture is then incubated until the cells reach an optical density suitable for use in the assay.

6.5.3 Selection of Medium to be Used in Assay

Two-fold dilution series of the inducer or repressor for the regulatable promoter which is linked to the gene required for the fungal proliferation, virulence or pathogenicity of the conditional-expression C. neoformans mutant strain are generated in culture medium containing the appropriate antibiotic for which the conditional-expression C. neoformans mutant strain contains a gene which confers resistance. Several medium are tested side by side and three to four wells are used to evaluate the effects of the inducer or repressor at each concentration in each media. Equal volumes of test media-inducer or repressor and conditional-expression C. neoformans mutant strain cells are added to the wells of a 384 well microtiter plate and mixed. The cells are prepared as described above and diluted in the appropriate medium containing the test antibiotic immediately prior to addition to the microtiter plate wells. For a control, cells are also added to several wells of each medium that do not contain inducer or repressor. Cell growth is monitored continuously by incubation by monitoring the optical density of the wells. The percent inhibition of growth produced by each concentration of inducer or repressor is calculated by comparing the rates of logarithmic growth against that exhibited by cells growing in medium without inducer or repressor. The medium yielding greatest sensitivity to inducer or repressor is selected for use in the assays described below.

6.5.4 Measurement of Test Antibiotic Sensitivity in Conditional-expression C. neoformans Mutant Strains in which the Level of the Target Gene Product is Not Rate Limiting

Two-fold dilution series of antibiotics of known mechanism of action are generated in the culture medium selected for further assay development that has been supplemented with the antibiotic used to maintain the conditional-expression C. neoformans mutant strain. A panel of test antibiotics known to act on different pathways is tested side by side with three to four wells being used to evaluate the effect of a test antibiotic on cell growth at each concentration. Equal volumes of test antibiotic and cells are added to the wells of a 384 well microtiter plate and mixed. Cells are prepared as described above using the medium selected for assay development supplemented with the antibiotic required to maintain the conditional-expression C. neoformans mutant strain and are diluted in identical medium immediately prior to addition to the microtiter plate wells. For a control, cells are also added to several wells that lack antibiotic, but contain the solvent used to dissolve the antibiotics. Cell growth is monitored continuously by incubation in a microtiter plate reader monitoring the optical density of the wells. The percent inhibition of growth produced by each concentration of antibiotic is calculated by comparing the rates of logarithmic growth against that exhibited by cells growing in medium without antibiotic. A plot of percent inhibition against log [antibiotic concentration] allows extrapolation of an IC₅₀ value for each antibiotic.

### 6.5 Measurement of Test Antibiotic Sensitivity in the Conditional-expression C. neoformans Mutant Strains in which the Level of the Target Gene Product is Rate Limiting

The culture medium selected for use in the assay is supplemented with inducer or repressor at concentrations shown to inhibit cell growth by a desired amount as described above, as well as the antibiotic used to maintain the conditional-expression C. neoformans mutant strain. Two fold dilution series of the panel of test antibiotics used above are generated in each of these media. Several antibiotics are tested side by side in each medium with three to four wells being used to evaluate the effects of an antibiotic on cell growth at each concentration. Equal volumes of test antibiotic and cells are added to the wells of a 384 well microtiter plate and mixed. Cells are prepared as described above using the medium selected for use in the assay supplemented with the antibiotic required to maintain the conditional-expression C. neoformans mutant strain. The cells are diluted 1:100 into two aliquots of identical medium containing concentrations of inducer that have been shown to inhibit cell growth by the desired amount and incubated under appropriate growth conditions. Immediately prior to addition to the microtiter plate wells, the cultures are adjusted to an appropriate optical density by dilution into warm sterile medium supplemented with identical concentrations of the inducer and antibiotic used to maintain the conditional-expression C. neoformans mutant strain. For a control, cells are also added to several wells that contain solvent used to dissolve test antibiotics but which contain no antibiotic. Cell growth is monitored continuously by incubation under suitable growth conditions in a microtiter plate reader monitoring the optical density of the wells. The percent inhibition of growth produced by each concentration of antibiotic is calculated by comparing the rates of logarithmic growth against that exhibited by cells growing in medium without antibiotic. A plot of percent inhibition against log [antibiotic concentration] allows extrapolation of an IC₅₀ value for each antibiotic.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A purified or isolated nucleic acid molecule comprising a nucleotide sequence encoding a polypeptide, wherein said polypeptide consists essentially of an amino acid sequence of one of SEQ ID NO: 3001-3361.

2. The nucleic acid molecule of claim 1, wherein said nucleotide sequence is one of SEQ ID NO: 2001-2361.

3. A nucleic acid molecule comprising a fragment of one of SEQ ID NO: 1-361 or 1001-1361, said fragment selected from the group consisting of fragments comprising at least 10, at least 20, at least 25, at least 30, at least 50 and at least 100 consecutive nucleotides of one of SEQ ID NO: 1-361 or 1001-1361.

4. A nucleic acid molecule comprising a nucleotide sequence that hybridizes under stringent condition to a second nucleic acid molecule consisting of (a) a nucleotide sequence selected from the group consisting of one of SEQ ID NO: 1-361 or SEQ ID NO: 1001-1361, or (b) a nucleotide sequence that encodes a polypeptide consisting of an amino acid sequence selected from the group consisting of one of SEQ ID NO: 3001-3361;
wherein said stringent condition comprises hybridization to filter-bound DNA in 6×sodium chloride/sodium citrate (SSC) at about 45° C. followed by one or more washes in 0.2×SSC/0.1% SDS at about 50-65° C.

5. A purified or isolated nucleic acid molecule obtained from an organism other than Aspergullus fumigatus, Candida albicans or Saccharomyces cerevisiae comprising a nucleotide sequence having at least 30% identity to a sequence selected from the group consisting of SEQ ID NO: 1-361 or 1001-1361; fragments comprising at least 25 consecutive nucleotides of SEQ ID NO: 1-361 or 1001-1361; the sequences complementary to SEQ ID NO: 1-361 or 1001-1361; and the sequences complementary to fragments comprising at least 25 consecutive nucleotides of SEQ ID NO: 1-361 or 1001-1361, as determined using BLASTN version 2.0 with default parameters.

6. A nucleic acid construct comprising a promoter operably linked to the nucleic acid molecule of claim 1, 2, 3, 4, or 5.

7. The nucleic acid construct of claim 6, which is a vector.

8. A host cell comprising the nucleic acid construct of claim 6.

9. A purified or isolated polypeptide comprising an amino acid sequence selected from the group consisting of one of SEQ ID NO: 3001-3361.

10. A purified or isolated polypeptide obtained from an organism other than Aspergullus fumigatus, Candida albicans or Saccharomyces cerevisiae comprising an amino acid sequence having at least 30% similarity to an amino acid sequence selected from the group consisting of one of SEQ ID NO: 3001-3361, as determined using FASTA version 3.0t78 with the default parameters.

11. A fusion protein comprising a fragment of a first polypeptide fused to a second polypeptide, said fragment consisting of at least 6 consecutive residues of an amino acid sequence selected from one of SEQ ID NO: 3001-3361.

12. A method of producing a polypeptide, said method comprises introducing into a cell, a nucleic acid construct comprising a promoter operably linked to a nucleotide sequence encoding a polypeptide consisting of an amino acid sequence selected from the group consisting of one of SEQ ID NO: 3001-3361; and culturing the cell such that the nucleotide sequence is expressed.

13. A method of producing a polypeptide, said method comprising providing a cell which comprises a heterologous promoter operably linked to a nucleotide sequence encoding a polypeptide consisting of an amino acid sequence selected from the group consisting of one of SEQ ID NO: 3001-3361; and culturing the cell such that the nucleotide sequence is expressed.

14. A method for identifying a compound which modulates the activity of a gene product encoded by (i) a nucleic acid comprising a nucleotide sequence selected from the group consisting of one of SEQ ID NO: 2001-2361 or (ii) a gene corresponding to a nucleotide sequence selected from the group consisting of one of SEQ ID NO: 2001-2361, said method comprising:
(a) contacting said gene product with a compound; and
(b) determining whether said compound modulates the activity of said gene product.

15. The method of claim 14, wherein the activity of the gene product is inhibited.

16. The method of claim 14, wherein said gene product is a polypeptide and said activity is selected from the group consisting of an enzymatic activity, carbon compound catabolism activity, a biosynthetic activity, a transporter activity, a transcriptional activity, a translational activity, a signal transduction activity, a DNA replication activity, and a cell division activity.

17. A method of eliciting an immune response in an animal, comprising introducing into the animal a composition comprising an isolated polypeptide, the amino acid sequence of which comprises at least 6 consecutive residues of one of SEQ ID NO: 3001-3361.

18. An isolated strain of Cryptococcus neoformans wherein the gene (i) comprising a nucleotide sequence selected from the group consisting of one of SEQ ID NO: 1-361, 1001-1361 and 2001-2361 or (ii) corresponding to a nucleotide sequence selected from the group consisting of one of SEQ ID NO: 1-361, 1001-1361 and 2001-2361; is inactive or placed under the control of a heterologous promoter.

19. An isolated strain of Cryptococcus neoformans comprising a nucleic acid molecule comprising a nucleotide sequence selected from one of SEQ ID NO: 1-361, 1001-1361 and 2001-2361, under the control of a heterologous promoter.

20. The strain of claim 18 or 19, wherein said heterologous promoter is regulatable.

21. A method of identifying a compound or binding partner that binds to (i) a polypeptide comprising an amino acid sequence selected from the group consisting of one of SEQ ID NO: 3001-3361, or (ii) a polypeptide encoded by a gene corresponding to a nucleotide sequence selected from one of SEQ ID NO: 2001-2361; or a a fragment thereof said method comprising:
(a) contacting the polypeptide or fragment thereof with a plurality of compounds or a preparation comprising one or more binding partners; and
(b) identifying a compound or binding partner that binds to the polypeptide or fragment thereof.

22. A method for identifying a compound having the ability to inhibit growth or proliferation of Cryptococcus neoformans, said method comprising the steps of:
(a) reducing the level or activity of a gene product encoded by (i) a nucleic acid selected from the group consisting of SEQ ID NO: 2001-2361 or (ii) a gene corresponding to a nucleic acid selected from the group consisting of SEQ ID NO: 2001-2361; in a Cryptococcus neoformans cell relative to a wild type cell, wherein said reduced level is not lethal to said cell;
(b) contacting said cell with a compound; and
(c) determining whether said compound inhibits the growth or proliferation of said cell.

23. The method of claim 22, wherein said step of reducing the level or activity of said gene product comprises transcribing a nucleotide sequence encoding said gene product from a regulatable promoter under conditions in which said gene product is expressed at said reduced level.

24. The method of claim 23, wherein said gene product is a polypeptide comprising a sequence selected from the group consisting of polypeptides encoded by SEQ ID NO: 3001-3361.

25. A method for inhibiting growth or proliferation of Cryptococcus neoformans cells comprising contacting the cells with a compound that (i) reduce the level of or inhibit the activity of a nucleotide sequence selected from the group consisting of SEQ ID NO: 1-361, 1001-1361 and 2001-2361 or (ii) reduce the level of or inhibit the activity of a gene product encoded by (a) a nucleotide sequence selected from the group consisting of SEQ ID NO: 1-361, 1001-1361 and 2001-2361 or (b) a gene corresponding to a nucleic acid selected from the group consisting of SEQ ID NO: 2001-2361.

26. The method of claim 25, wherein said gene product is a polypeptide comprising an amino acid sequence selected from the group consisting of polypeptides encoded by SEQ ID NO: 3001-3361.

27. The method of claim 25, wherein the compound is an antibody, a fragment of an antibody, an antisense nucleic acid molecule, or a ribozyme.

28. A method for manufacturing an antimycotic compound comprising the steps of:
(a) screening a plurality of candidate compounds to identify a compound that reduces the activity or level of a gene product encoded by (i) a nucleotide sequence selected from the group consisting of SEQ ID NO: 1-361, 1001-1361 and 2001-2361 or (ii) a gene corresponding to a nucleic acid selected from the group consisting of SEQ ID NO: 2001-2361; and
(b) manufacturing the compound so identified.

29. The method of claim 28, wherein said gene product is a polypeptide comprising an amino acid sequence selected from the group consisting of polypeptides encoded by SEQ ID NO: 1-361, 1001-1361 and 2001-2361.

30. A method for treating an infection of a subject by Cryptococcus neoformans comprising administering a pharmaceutical composition comprising a therapeutically effective amount of a compound that reduces the activity or level of a gene product encoded by (i) a nucleic acid comprising a sequence selected from the group consisting of SEQ ID NO: 1-361, 1001-1361 and 2001-2361 or (ii) a gene corresponding to a nucleic acid selected from the group consisting of SEQ ID NO: 2001-2361, and a pharmaceutically acceptable carrier, to said subject.

31. The method of claim 30, wherein the compound is an antibody, a fragment of an antibody, an antisense nucleic acid molecule, or a ribozyme.

32. A method for preventing or containing contamination of an object by Cryptococcus neoformans comprising contacting the object with a composition comprising an effective amount of a compound that reduces the activity or level of a gene product encoded by (i) a nucleic acid comprising a sequence selected from the group consisting of SEQ ID NO: 1-361, 1001-1361 and 2001-2361 or (ii) a gene corresponding to a nucleic acid selected from the group consisting of SEQ ID NO: 2001-2361.

33. A pharmaceutical composition comprising a therapeutically effective amount of an agent which reduces the activity or level of a gene product encoded by (i) a nucleic acid selected from the group consisting of SEQ ID NO: 1-361, 1001-1361 and 2001-2361 or (ii) a gene corresponding to a nucleic acid selected from the group consisting of SEQ ID NO: 2001-2361, in a pharmaceutically acceptable carrier.

34. The method of claim 30, wherein said subject is selected from the group consisting of a plant, a vertebrate, a mammal, an avian, and a human.

35. An antibody preparation which binds the polypeptide of claim 9 or 10.

36. The antibody preparation of claim 35 which comprises a monoclonal antibody.

37. A method for evaluating a compound against a target gene product encoded by (i) a nucleotide sequence comprising one of SEQ ID NO: 1-361, 1001-1361 and 2001-2361 or (ii) a gene corresponding to a nucleic acid selected from the group consisting of SEQ ID NO: 2001-2361, said method comprising the steps of:
(a) contacting wild type fungal cells with the compound and generating a first protein expression profile;
(b) determining the protein expression profile of the fungal cells of claim 18 or 19, which have been cultured under conditions wherein the target gene is substantially underexpressed, not expressed or overexpressed and generating a second protein expression profile for the cultured cells; and
(c) comparing the first protein expression profile with the second protein expression profile to identify similarities in the profiles.

38. A collection of Cryptococcus neoformans strains of claim 18 or 19, wherein the cells of each strain further comprises one or more molecular tags each of about 20 nucleotides, wherein the sequences of each tag in a cell is unique to the strain of cells.

39. The collection of claim 38, wherein the molecular tag(s) is disposed within the gene disruption cassette.

40. A nucleic acid molecule array comprising a plurality of nucleic acid molecules, wherein at least one nucleic acid molecule comprises a nucleotide sequence that is hybridizable to a target nucleotide sequence or a complement thereof, said target nucleotide sequence being selected from the group consisting of SEQ ID NO: 1-361, 1001-1361, and 2001-2361.

41. A protein array comprising a plurality of proteins, wherein at least one protein comprises an amino acid sequence or a portion of an amino acid sequence selected from the group consisting of SEQ ID NO:3001 through to SEQ ID NO:3361.

42. A computer or a computer readable medium that comprises at least one nucleotide sequence selected from the group consisting of SEQ ID NO: 1001-1361 and SEQ ID NO: 2001-2361, or at least one amino acid sequence selected from the group consisting of SEQ ID NO: 3001-3361.

43. A method assisted by a computer for identifying a putatively essential gene of a fungus, comprising detecting sequence homology between a fungal nucleotide sequence or fungal amino acid sequence with at least one nucleotide sequence selected from the group consisting of SEQ ID NO: 1001-1361 and SEQ ID NO: 2001-2361, or at least one amino acid sequence selected from the group consisting of SEQ ID NO: 3001-3361.
